SEQUENCE LISTING (166 sequences)

SEQ ID NO: 1 - protein, 8 aa, Artificial Sequence
  Heavy chain variable region CDR
GYTFTTNG

SEQ ID NO: 2 - protein, 8 aa, Artificial Sequence
  Heavy chain variable region CDR
INTNTGEP

SEQ ID NO: 3 - protein, 10 aa, Artificial Sequence
  Heavy chain variable region CDR
AREGAFGLDY

SEQ ID NO: 4 - protein, 8 aa, Artificial Sequence
  Heavy chain variable region CDR
DYTFTNYW

SEQ ID NO: 5 - protein, 8 aa, Artificial Sequence
  Heavy chain variable region CDR
IYPGGGYT

SEQ ID NO: 6 - protein, 12 aa, Artificial Sequence
  Heavy chain variable region CDR
ARVRSGNDALDF

SEQ ID NO: 7 - protein, 8 aa, Artificial Sequence
  Heavy chain variable region CDR
GYTFTNYG

SEQ ID NO: 8 - protein, 8 aa, Artificial Sequence
  Heavy chain variable region CDR
INTNTGEP

SEQ ID NO: 9 - protein, 10 aa, Artificial Sequence
  Heavy chain variable region CDR
SREGIYGMDS

SEQ ID NO: 10 - protein, 8 aa, Artificial Sequence
  Heavy chain variable region CDR
GFSLTSYG

SEQ ID NO: 11 - protein, 7 aa, Artificial Sequence
  Heavy chain variable region CDR
IWAGGRT

SEQ ID NO: 12 - protein, 14 aa, Artificial Sequence
  Heavy chain variable region CDR
AREGGYDYDGDLWT

SEQ ID NO: 13 - protein, 8 aa, Artificial Sequence
  Heavy chain variable region CDR
GFSLTSYG

SEQ ID NO: 14 - protein, 7 aa, Artificial Sequence
  Heavy chain variable region CDR
IWSGGIT

SEQ ID NO: 15 - protein, 13 aa, Artificial Sequence
  Heavy chain variable region CDR
ARNFDGYYYSIDY

SEQ ID NO: 16 - protein, 8 aa, Artificial Sequence
  Heavy chain variable region CDR
GFSLTNYG

SEQ ID NO: 17 - protein, 7 aa, Artificial Sequence
  Heavy chain variable region CDR
IWRGGST

SEQ ID NO: 18 - protein, 12 aa, Artificial Sequence
  Heavy chain variable region CDR
AKNLYGPAAMDY

SEQ ID NO: 19 - protein, 8 aa, Artificial Sequence
  Heavy chain variable region CDR
GDTFTNYA

SEQ ID NO: 20 - protein, 8 aa, Artificial Sequence
  Heavy chain variable region CDR
IDTYTGDP

SEQ ID NO: 21 - protein, 8 aa, Artificial Sequence
  Heavy chain variable region CDR
AGDTWFAY

SEQ ID NO: 22 - protein, 10 aa, Artificial Sequence
  Heavy chain variable region CDR
GFSLSSSGMS

SEQ ID NO: 23 - protein, 7 aa, Artificial Sequence
  Heavy chain variable region CDR
IWWSDDK

SEQ ID NO: 24 - protein, 12 aa, Artificial Sequence
  Heavy chain variable region CDR
AREGSLGYGLDY

SEQ ID NO: 25 - protein, 8 aa, Artificial Sequence
  Heavy chain variable region CDR
GFDFRRYW

SEQ ID NO: 26 - protein, 8 aa, Artificial Sequence
  Heavy chain variable region CDR
INPDSSMI

SEQ ID NO: 27 - protein, 10 aa, Artificial Sequence
  Heavy chain variable region CDR
ATYGHHATDS

SEQ ID NO: 28 - protein, 8 aa, Artificial Sequence
  Heavy chain variable region CDR
GYTFTSYW

SEQ ID NO: 29 - protein, 8 aa, Artificial Sequence
  Heavy chain variable region CDR
IYPGSDST

SEQ ID NO: 30 - protein, 11 aa, Artificial Sequence
  Heavy chain variable region CDR
ALDSSGYGFAY

SEQ ID NO: 31 - protein, 8 aa, Artificial Sequence
  Heavy chain variable region CDR
DYEFPSHD

SEQ ID NO: 32 - protein, 8 aa, Artificial Sequence
  Heavy chain variable region CDR
INSDGGNT

SEQ ID NO: 33 - protein, 16 aa, Artificial Sequence
  Heavy chain variable region CDR
ARPRPTIGTTATGSSM

SEQ ID NO: 34 - protein, 6 aa, Artificial Sequence
  Light chain variable region CDR
QSVSND

SEQ ID NO: 35 - protein, 3 aa, Artificial Sequence
  Light chain variable region CDR
YGS

SEQ ID NO: 36 - protein, 9 aa, Artificial Sequence
  Light chain variable region CDR
QQDFFSPWT

SEQ ID NO: 37 - protein, 10 aa, Artificial Sequence
  Light chain variable region CDR
ESVDEYGISF

SEQ ID NO: 38 - protein, 3 aa, Artificial Sequence
  Light chain variable region CDR
SAS

SEQ ID NO: 39 - protein, 9 aa, Artificial Sequence
  Light chain variable region CDR
QQSKEVPWT

SEQ ID NO: 40 - protein, 6 aa, Artificial Sequence
  Light chain variable region CDR
QSVSND

SEQ ID NO: 41 - protein, 3 aa, Artificial Sequence
  Light chain variable region CDR
YAS

SEQ ID NO: 42 - protein, 9 aa, Artificial Sequence
  Light chain variable region CDR
QQDYYSPWT

SEQ ID NO: 43 - protein, 11 aa, Artificial Sequence
  Light chain variable region CDR
QSLVHSNGNTY

SEQ ID NO: 44 - protein, 3 aa, Artificial Sequence
  Light chain variable region CDR
KVS

SEQ ID NO: 45 - protein, 9 aa, Artificial Sequence
  Light chain variable region CDR
SQSTHVPPD

SEQ ID NO: 46 - protein, 6 aa, Artificial Sequence
  Light chain variable region CDR
QDISNS

SEQ ID NO: 47 - protein, 3 aa, Artificial Sequence
  Light chain variable region CDR
YTS

SEQ ID NO: 48 - protein, 9 aa, Artificial Sequence
  Light chain variable region CDR
QQGNTLPLT

SEQ ID NO: 49 - protein, 6 aa, Artificial Sequence
  Light chain variable region CDR
QNIYVW

SEQ ID NO: 50 - protein, 3 aa, Artificial Sequence
  Light chain variable region CDR
EAS

SEQ ID NO: 51 - protein, 9 aa, Artificial Sequence
  Light chain variable region CDR
QQGQSYPYT

SEQ ID NO: 52 - protein, 12 aa, Artificial Sequence
  Light chain variable region CDR
QSLLYSRNQKNR

SEQ ID NO: 53 - protein, 3 aa, Artificial Sequence
  Light chain variable region CDR
WAS

SEQ ID NO: 54 - protein, 9 aa, Artificial Sequence
  Light chain variable region CDR
QQYYSYPRT

SEQ ID NO: 55 - protein, 11 aa, Artificial Sequence
  Light chain variable region CDR
QSIVHTDGNTY

SEQ ID NO: 56 - protein, 3 aa, Artificial Sequence
  Light chain variable region CDR
KVS

SEQ ID NO: 57 - protein, 9 aa, Artificial Sequence
  Light chain variable region CDR
FQGSHVPWT

SEQ ID NO: 58 - protein, 10 aa, Artificial Sequence
  Light chain variable region CDR
ESVDSYGNSF

SEQ ID NO: 59 - protein, 3 aa, Artificial Sequence
  Light chain variable region CDR
RAS

SEQ ID NO: 60 - protein, 9 aa, Artificial Sequence
  Light chain variable region CDR
QQSNEDPFT

SEQ ID NO: 61 - protein, 6 aa, Artificial Sequence
  Light chain variable region CDR
QSVSND

SEQ ID NO: 62 - protein, 3 aa, Artificial Sequence
  Light chain variable region CDR
YAS

SEQ ID NO: 63 - protein, 9 aa, Artificial Sequence
  Light chain variable region CDR
QQDYSSPFT

SEQ ID NO: 64 - protein, 10 aa, Artificial Sequence
  Light chain variable region CDR
QSVSTSTYNY

SEQ ID NO: 65 - protein, 3 aa, Artificial Sequence
  Light chain variable region CDR
YAS

SEQ ID NO: 66 - protein, 9 aa, Artificial Sequence
  Light chain variable region CDR
QHSWEIPFT

SEQ ID NO: 67 - protein, 117 aa, Artificial Sequence
  Heavy chain variable region
QIQLVQSGPELKKPGETVKISCKASGYTFTTNGINWVKQAAGKGLKWMGWINTNTGEPTNAEELKGRFAFSLETSASTAYLQINNLKNEDAAIYFCAREGAFGLDYWGQGTSVTVSS

SEQ ID NO: 68 - protein, 119 aa, Artificial Sequence
  Heavy chain variable region
QVQLQQSGAELVRPGTSAKISCKASDYTFTNYWLGWLKQRPGHGLEWIGHIYPGGGYTNYNEKFKGKATLTADTSSSTAYMQLSSLTSEDSAVYFCARVRSGNDALDFWGQGTSVTVSS

SEQ ID NO: 69 - protein, 117 aa, Artificial Sequence
  Heavy chain variable region
QIQLVQSGPELKKPGETVKISCKASGYTFTNYGMNWVKQAPGKGLKWMGWINTNTGEPTYAEEIKGRFAFSLETSASTAYLQINNLKNEDTATYFCSREGIYGMDSWGQGTSVTVSS

SEQ ID NO: 70 - protein, 121 aa, Artificial Sequence
  Heavy chain variable region
QVQLKESGPGLVAPSQSLSITCTVSGFSLTSYGVHWVRQPPGKGLEWLGIIWAGGRTDYNSALVSRLNISKDNSKSQVFLKMHSLQTDDTAMYYCAREGGYDYDGDLWTTGVKEPQSPSPQ

SEQ ID NO: 71 - protein, 119 aa, Artificial Sequence
  Heavy chain variable region
QVQLKQSGPGLVLPSQSLSITCTVSGFSLTSYGVHWFRQSPGTGLEWLGVIWSGGITDYNATFIPRLSISKDNSKSQVFFKMNSLQADDTAIYYCARNFDGYYYSIDYWGQGTSVTVSS

SEQ ID NO: 72 - protein, 118 aa, Artificial Sequence
  Heavy chain variable region
QVQLKQSGPGLVQPSQSLSITCTVSGFSLTNYGVHWVRQSPGKGLEWLGAIWRGGSTDYNAAFMSRLSITKDNSKSQVFFKMNSLQPDDTAIYYCAKNLYGPAAMDYWGQGTSVTVSS

SEQ ID NO: 73 - protein, 115 aa, Artificial Sequence
  Heavy chain variable region
QIQLVQSGPELKKPGETVNISCRASGDTFTNYAVNWVKQAPGKGLKWMGWIDTYTGDPTYADDLKGRFAFSLETSATTAYLQINNLKNEDTATYFCAGDTWFAYWGQGTLVTVSA

SEQ ID NO: 74 - protein, 120 aa, Artificial Sequence
  Heavy chain variable region
QVTLKESGPGILQPSQTLSLTCSFSGFSLSSSGMSVGWIRQPSGKGLEWLAHIWWSDDKSYNPALKSRLTISKDTSNNQVFLKIASVVTADTATYYCAREGSLGYGLDYWGQGTTLTVSS

SEQ ID NO: 75 - protein, 117 aa, Artificial Sequence
  Heavy chain variable region
EVKLLESGGGLVQPGGSLKLSCAASGFDFRRYWMSWVRQAPGKGLEWIGQINPDSSMINYTPSLKDKFIISRDNAKNTLYLQMSEVRSEDTALYYCATYGHHATDSWGQGTSVTVSS

SEQ ID NO: 76 - protein, 118 aa, Artificial Sequence
  Heavy chain variable region
QVQLQQPGAELVRPGTSVKMSCKASGYTFTSYWMHWVKQRPGQGLEWIGDIYPGSDSTNYHEKFKSKATLTVDTSSSTAYMQLSSLTSEDSAVYYCALDSSGYGFAYWGQGTLVTVSA

SEQ ID NO: 77 - protein, 120 aa, Artificial Sequence
  Heavy chain variable region
EVQLVESGGGLVQPGESLKLSCDSNDYEFPSHDMSWVRKTPEKRLELVAAINSDGGNTYYPDTMERRFIISRDNTKRTLYLQMSSLRSEDTALHYCARPRPTIGTTATGSSMSGAQGPRS

SEQ ID NO: 78 - protein, 107 aa, Artificial Sequence
  Light chain variable region
SIVMTQSPKFLLVSAGDRVIITCKASQSVSNDVVWYQQKPGQSPKLLIYYGSHRNTGVPTRFTGSGYGTDFTFTISTVQAEDLAVYFCQQDFFSPWTFGGGTKLEIK

SEQ ID NO: 79 - protein, 111 aa, Artificial Sequence
  Light chain variable region
DIVLTQSPASLAVSLGQRATISCRASESVDEYGISFMNWFQQKPGQPPRLLIYSASNQGSGVPARFTGSGSGTDFSLNILPMEEDDTAMYFCQQSKEVPWTFGGGTKLEIK

SEQ ID NO: 80 - protein, 107 aa, Artificial Sequence
  Light chain variable region
SIVMTQTPKFLLVSAGDRVTITCKASQSVSNDVVWYRQKPGQSPKLLIYYASNRYIGVPDRFTGSGYGTDFTFTISTVQAEDLAVYFCQQDYYSPWTFGGGTKLEIK

SEQ ID NO: 81 - protein, 112 aa, Artificial Sequence
  Light chain variable region
DVVMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPDVRWRHQAGNQ

SEQ ID NO: 82 - protein, 107 aa, Artificial Sequence
  Light chain variable region
DIQMTQTTSSLSASLGDRVTISCRASQDISNSLNWYQQKPDGTVKLLIYYTSRLHSGVPSRFTGSGSGTDYSLTISNLEQEDIATYFCQQGNTLPLTFGAGTKLELK

SEQ ID NO: 83 - protein, 107 aa, Artificial Sequence
  Light chain variable region
DIQMNQSPSSLSASLGDTITITCHANQNIYVWLSWYQQKPGNIPKLLIYEASNLHTGVPSRFSGSGSGTGFTLTISSLQPEDIATYYCQQGQSYPYTFGGGTKLEIK

SEQ ID NO: 84 - protein, 113 aa, Artificial Sequence
  Light chain variable region
DIVMSQSPSSLAVSVGEKVTMSCKSSQSLLYSRNQKNRLAWYQQKPGQSPKLLIYWASTRESGVPDRFTGSGSGTDFTLTISSVKAEDLAVYYCQQYYSYPRTFGGGTNLEIK

SEQ ID NO: 85 - protein, 112 aa, Artificial Sequence
  Light chain variable region
DVLMTQTPLSLPVSLGDQASISCRSSQSIVHTDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 86 - protein, 111 aa, Artificial Sequence
  Light chain variable region
DIVLTQSPASLAVSLGQRATISCRASESVDSYGNSFMHWYQQKPGQPPKLLIYRASNLESGIPARFSGSGSRTDFTLTINPVEADDVATYYCQQSNEDPFTFGSGTKLEIK

SEQ ID NO: 87 - protein, 107 aa, Artificial Sequence
  Light chain variable region
NIVMTQTPKFLLVSAGDRVTITRKASQSVSNDVVWYQQKPGQSPKLLMYYASYRYTGVPDRFTGSGYGTDFTFTISNVQAEDLAVYFCQQDYSSPFTFGSGTKLEIK

SEQ ID NO: 88 - protein, 111 aa, Artificial Sequence
  Light chain variable region
DIVLTQSPASLAVSLGQRATISCRASQSVSTSTYNYMHWYQQKPGQPPKLLIKYASNLESGVPARFSGSGSGTDFTLNIHPVEEEDTATYYCQHSWEIPFTFGSGTKLEIK

SEQ ID NO: 89 - protein, 330 aa, Artificial Sequence [sequence omitted]

SEQ ID NO: 90 - protein, 107 aa, Artificial Sequence [sequence omitted]

SEQ ID NO: 91 - protein, 447 aa, Artificial Sequence
  Heavy chain
QIQLVQSGPELKKPGETVKISCKASGYTFTTNGINWVKQAAGKGLKWMGWINTNTGEPTNAEELKGRFAFSLETSASTAYLQINNLKNEDAAIYFCAREGAFGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 449 aa, Artificial Sequence
  Heavy chain
QVQLQQSGAELVRPGTSAKISCKASDYTFTNYWLGWLKQRPGHGLEWIGHIYPGGGYTNYNEKFKGKATLTADTSSSTAYMQLSSLTSEDSAVYFCARVRSGNDALDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 447 aa, Artificial Sequence
  Heavy chain
QIQLVQSGPELKKPGETVKISCKASGYTFTNYGMNWVKQAPGKGLKWMGWINTNTGEPTYAEEIKGRFAFSLETSASTAYLQINNLKNEDTATYFCSREGIYGMDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 451 aa, Artificial Sequence
  Heavy chain
QVQLKESGPGLVAPSQSLSITCTVSGFSLTSYGVHWVRQPPGKGLEWLGIIWAGGRTDYNSALVSRLNISKDNSKSQVFLKMHSLQTDDTAMYYCAREGGYDYDGDLWTTGVKEPQSPSPQASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 449 aa, Artificial Sequence
  Heavy chain
QVQLKQSGPGLVLPSQSLSITCTVSGFSLTSYGVHWFRQSPGTGLEWLGVIWSGGITDYNATFIPRLSISKDNSKSQVFFKMNSLQADDTAIYYCARNFDGYYYSIDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 448 aa, Artificial Sequence
  Heavy chain
QVQLKQSGPGLVQPSQSLSITCTVSGFSLTNYGVHWVRQSPGKGLEWLGAIWRGGSTDYNAAFMSRLSITKDNSKSQVFFKMNSLQPDDTAIYYCAKNLYGPAAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 445 aa, Artificial Sequence
  Heavy chain
QIQLVQSGPELKKPGETVNISCRASGDTFTNYAVNWVKQAPGKGLKWMGWIDTYTGDPTYADDLKGRFAFSLETSATTAYLQINNLKNEDTATYFCAGDTWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 450 aa, Artificial Sequence
  Heavy chain
QVTLKESGPGILQPSQTLSLTCSFSGFSLSSSGMSVGWIRQPSGKGLEWLAHIWWSDDKSYNPALKSRLTISKDTSNNQVFLKIASVVTADTATYYCAREGSLGYGLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 447 aa, Artificial Sequence
  Heavy chain
EVKLLESGGGLVQPGGSLKLSCAASGFDFRRYWMSWVRQAPGKGLEWIGQINPDSSMINYTPSLKDKFIISRDNAKNTLYLQMSEVRSEDTALYYCATYGHHATDSWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 448 aa, Artificial Sequence
  Heavy chain
QVQLQQPGAELVRPGTSVKMSCKASGYTFTSYWMHWVKQRPGQGLEWIGDIYPGSDSTNYHEKFKSKATLTVDTSSSTAYMQLSSLTSEDSAVYYCALDSSGYGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 450 aa, Artificial Sequence
  Heavy chain
EVQLVESGGGLVQPGESLKLSCDSNDYEFPSHDMSWVRKTPEKRLELVAAINSDGGNTYYPDTMERRFIISRDNTKRTLYLQMSSLRSEDTALHYCARPRPTIGTTATGSSMSGAQGPRSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 214 aa, Artificial Sequence
  Light chain
SIVMTQSPKFLLVSAGDRVIITCKASQSVSNDVVWYQQKPGQSPKLLIYYGSHRNTGVPTRFTGSGYGTDFTFTISTVQAEDLAVYFCQQDFFSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 218 aa, Artificial Sequence
  Light chain
DIVLTQSPASLAVSLGQRATISCRASESVDEYGISFMNWFQQKPGQPPRLLIYSASNQGSGVPARFTGSGSGTDFSLNILPMEEDDTAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 214 aa, Artificial Sequence
  Light chain
SIVMTQTPKFLLVSAGDRVTITCKASQSVSNDVVWYRQKPGQSPKLLIYYASNRYIGVPDRFTGSGYGTDFTFTISTVQAEDLAVYFCQQDYYSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 219 aa, Artificial Sequence
  Light chain
DVVMTQSPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPDVRWRHQAGNQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 214 aa, Artificial Sequence
  Light chain
DIQMTQTTSSLSASLGDRVTISCRASQDISNSLNWYQQKPDGTVKLLIYYTSRLHSGVPSRFTGSGSGTDYSLTISNLEQEDIATYFCQQGNTLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 214 aa, Artificial Sequence
  Light chain
DIQMNQSPSSLSASLGDTITITCHANQNIYVWLSWYQQKPGNIPKLLIYEASNLHTGVPSRFSGSGSGTGFTLTISSLQPEDIATYYCQQGQSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 220 aa, Artificial Sequence
  Light chain
DIVMSQSPSSLAVSVGEKVTMSCKSSQSLLYSRNQKNRLAWYQQKPGQSPKLLIYWASTRESGVPDRFTGSGSGTDFTLTISSVKAEDLAVYYCQQYYSYPRTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 219 aa, Artificial Sequence
  Light chain
DVLMTQTPLSLPVSLGDQASISCRSSQSIVHTDGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 218 aa, Artificial Sequence
  Light chain
DIVLTQSPASLAVSLGQRATISCRASESVDSYGNSFMHWYQQKPGQPPKLLIYRASNLESGIPARFSGSGSRTDFTLTINPVEADDVATYYCQQSNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, Artificial Sequence
  Light chain
NIVMTQTPKFLLVSAGDRVTITRKASQSVSNDVVWYQQKPGQSPKLLMYYASYRYTGVPDRFTGSGYGTDFTFTISNVQAEDLAVYFCQQDYSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 218 aa, Artificial Sequence
  Light chain
DIVLTQSPASLAVSLGQRATISCRASQSVSTSTYNYMHWYQQKPGQPPKLLIKYASNLESGVPARFSGSGSGTDFTLNIHPVEEEDTATYYCQHSWEIPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - DNA, 1341 bp, Artificial Sequence
  Heavy chain nucleic acid
cagattcaattggtacaaagcggtcccgagctcaagaaacctggggagactgtgaaaatttcctgtaaggcctctggctacaccttcaccactaacggtatcaactgggtgaagcaggctgcaggaaaggggctcaagtggatgggatggatcaacactaatactggagaacccaccaatgctgaggaacttaaaggcagattcgccttctctcttgagacttcagccagcaccgcctacctccaaattaacaatctgaaaaatgaggatgccgctatctatttttgtgcccgagagggtgcattcggtcttgattactggggtcagggcacctctgtgacagtgtctagcgcctcaactaaggggcccagtgttttcccccttgccccttcctcaaaatcaactagcggaggcacagccgctcttggctgtctcgtgaaagattacttccccgagccagtgacagtctcttggaacagcggggcactcacctccggcgttcacacttttcccgcagtcctccagtctagcggattgtatagcctgtcttctgttgtcacagtgccatcctctagccttggcacccagacctacatttgtaatgtgaatcataagccttcaaacacaaaagtcgataaaaaagtggagccaaagagctgtgacaagactcacacttgccctccatgccctgccccagaactccttggcggacctagcgtcttcctgtttcctcctaagccaaaggacaccctgatgatctcacgcactcctgaagtgacctgcgttgtggtagacgtgagccacgaggatccagaggtgaagttcaactggtatgtggatggagtcgaggtgcataatgccaaaacaaaacctagggaggagcagtacaactctacctaccgggtggtgtctgttttgaccgttctgcaccaagactggttgaatggcaaggagtacaagtgcaaggtgtcaaacaaagcactcccagcccctatagagaagaccatcagcaaagctaaggggcagccaagagaaccacaggtttacaccctgcccccctcacgggatgaactgacaaagaaccaggtgtctctgacttgcctcgtgaaaggattctacccttcagatatcgcagtggagtgggagtctaacggccaacccgagaataattacaagacaactcccccagtcctggacagtgatgggtcttttttcctgtattctaagctcactgttgacaaaagccgctggcagcagggtaatgtattctcctgctccgtcatgcacgaagccctgcacaaccactatacccagaagtctctgagcttgtcacccggcaag

SEQ ID NO: 114 - DNA, 1347 bp, Artificial Sequence
  Heavy chain nucleic acid
caagtacagttgcagcagtctggagctgagctggttagacctggaaccagtgccaagatctcctgcaaagctagtgattatactttcacaaactactggctcggttggctgaaacaaagacccgggcacggtctggaatggataggccacatttacccaggcggcggatacaccaactataacgagaagtttaagggaaaggctactctgacagccgacacctcatccagcacagcctacatgcagcttagtagtttgaccagtgaggattccgcagtgtacttctgtgcacgtgtgaggtcagggaatgacgccctggacttctggggccagggtacttcagtgacagtgtcctccgctagcactaagggaccaagcgtgtttcctctggctccaagctctaaatcaacctcaggcggaaccgccgctttgggatgtcttgtgaaggattacttccctgagcctgtcaccgtctcctggaatagcggcgccctgacttccggggtgcatacattccctgccgtattgcagtcaagcggcctgtatagccttagctctgttgtaactgtgccatcttcttcccttggcacacagacctacatctgtaacgtgaaccataagccatccaatacaaaagttgataagaaggtggagccaaaatcatgtgacaaaactcatacatgccctccttgtccagcacctgagctgttgggcggaccaagtgtctttctgttccccccaaagccaaaggacacactgatgatcagtcgcacaccagaagtgacttgcgttgttgtcgatgtgagccacgaagaccctgaggttaagtttaactggtacgtggatggtgtggaggtgcacaatgccaagactaagccacgtgaagaacagtataactccacctatcgtgtggtctctgtcctgacagtactgcaccaggactggctcaatggaaaagagtacaagtgcaaggttagtaacaaggccttgcccgctcctatcgagaaaactatcagtaaggcaaagggacagccaagagaaccacaggtttacaccctgccacctagccgggacgaattgactaaaaaccaagtgagtctcacatgcctcgtcaagggattctacccatctgacattgccgtagagtgggagtccaatgggcagcccgagaataattataagactactccacctgtcctggattctgatggatccttctttttgtattccaagcttaccgtggacaaaagcaggtggcagcagggcaatgtcttttcatgcagcgtgatgcacgaagcacttcacaatcactacacacagaagagtctgtctctcagtcctggtaag

SEQ ID NO: 115 - DNA, 1341 bp, Artificial Sequence
  Heavy chain nucleic acid
caaattcagctggtgcagtccggcccagaacttaagaaacccggtgaaaccgtgaagatatcctgcaaagcttcaggctatacctttaccaactatggcatgaattgggtgaagcaagctcccgggaagggccttaaatggatgggctggatcaacaccaacacaggtgagccaacctatgccgaagaaatcaaaggacgctttgctttctctctcgaaactagcgcaagtaccgcttatttgcagatcaataatctgaaaaacgaggacaccgctacctacttttgctcaagagagggcatttacggcatggattcttggggccagggaacatctgtcaccgtctcatctgcatctaccaagggccctagtgtgttcccactggccccttcatccaagtccacttctggaggaacagccgccttgggctgtctcgttaaggactacttccccgagcctgttaccgtgtcctggaacagtggcgccctgacctccggtgttcacactttccccgctgtgttgcagtcctctggcctttattccctttcctctgtagtgacagtgcccagttccagtctgggtacacagacctacatttgcaatgtcaaccataaaccaagtaataccaaggtagataaaaaggtagagcctaagtcatgcgataaaactcacacatgtccaccatgtcctgctccagaactcttgggcggtccaagtgtctttttgttcccaccaaaacctaaggataccctcatgatctctaggacccccgaggtcacatgtgtggtggtcgacgttagtcacgaggatcccgaggttaaatttaactggtatgtcgatggggttgaagtccacaatgccaaaaccaaaccaagagaggaacagtacaattccacttaccgtgtggtttcagtgctgaccgtccttcatcaggactggctcaacgggaaggagtacaagtgcaaggtcagtaacaaggctcttccagcccccatcgaaaagactatttccaaagccaagggacaaccaagagagccacaagtgtacactcttccccccagcagggacgaactcacaaagaatcaggtttctctcacctgtctggttaagggcttttaccccagcgacatcgcagttgagtgggagagcaatggccagcctgagaataactataagacaacaccccccgtgctggattctgacggcagctttttcttgtactctaaactcactgtagacaaatccaggtggcagcaaggcaacgtgttttcctgctctgtcatgcacgaggcattgcacaatcactatactcagaaatcacttagcctgtcccccggtaag

SEQ ID NO: 116 - DNA, 1353 bp, Artificial Sequence
  Heavy chain nucleic acid
caagtacaattgaaagagagtggaccaggacttgtggcccctagccagtctctgtcaataacctgtactgtcagtggattttcccttaccagctatggggtacactgggttaggcagccacctggtaaaggacttgagtggctgggcattatttgggccggagggcggacagactataattctgcactggtatccagactcaatatcagtaaggacaacagcaaaagtcaagtgtttctgaaaatgcattcacttcaaaccgatgacaccgctatgtactactgtgctagggaaggcggttatgactatgatggagatctctggactaccggagtcaaggagcctcaaagccccagtcctcaggcctccacaaaagggcctagtgttttccccctggccccatcatctaagagtacatcaggaggaacagcagcccttggatgcctggtgaaagactactttccagaacccgttaccgtgtcctggaactcaggcgcacttacctctggcgtccatacatttcccgccgtattgcagagcagcggactgtactcattgtcatccgtggtgacagtgcctagtagttctcttggtacacagacctacatatgtaacgtgaatcataaaccctcaaatactaaagtcgacaagaaggttgagcccaagagctgtgataaaacccacacttgtcccccatgcccagcacctgaactgctcggaggcccatccgtctttctcttccctcctaagcctaaggatacacttatgatttcacggacccctgaagtgacatgtgtggtcgttgatgtgtcccatgaggacccagaagttaaatttaactggtatgtcgatggcgtggaggtgcacaacgccaagacaaagcccagggaagaacagtataattcaacataccgcgtcgttagcgtgctgacagttctgcatcaggattggcttaacggtaaagagtacaagtgcaaggtctccaacaaagctctgcctgcacctattgaaaaaactatatccaaggctaagggccagcctcgcgagcctcaggtatatacactgccacccagcagggacgaactgacaaagaatcaggtcagtctgacttgcctcgtcaagggattctacccttcagacatcgccgtggagtgggagtcaaacggtcagcccgagaacaactacaagacaacaccccctgtactggactccgacggctcctttttcctgtattctaagctgactgtcgataaatctcgctggcagcagggaaacgtcttttcctgctctgtgatgcacgaggctctgcacaatcactatactcagaagtcactgtccttgtctcccggaaag

SEQ ID NO: 117 - DNA, 1347 bp, Artificial Sequence
  Heavy chain nucleic acid
caggtacaattgaaacaaagtgggccaggtcttgtccttccttcccagtctctgagtattacctgtaccgtcagtggatttagccttacaagctatggagtccattggttcaggcaatccccaggcacaggcctggaatggttgggggtcatctggagtggtggtatcaccgattacaatgcaacctttatcccccgtctgtccatatcaaaggacaactctaagtcccaggtgtttttcaaaatgaactctctccaggcagatgacacagccatatattactgtgctcgtaacttcgacggctactactattccatagattattggggccagggcacttcagtcaccgtcagtagcgccagcactaagggtccttccgtttttccccttgctccttcttcaaagagcacaagtggggggactgcagctttgggctgcttggtgaaggattattttcctgagcctgtcacagtctcttggaatagcggggcactcacaagcggtgttcatacctttccagccgtgctgcagagttctggtctgtatagtctctcctcagtggtgaccgtaccttcttcctctctgggcacccagacatacatatgcaatgttaatcacaagccctccaacacaaaagtggataaaaaagttgagcctaagagttgtgacaaaactcacacatgtcctccttgcccagctcccgaactccttggaggaccaagtgtttttttgtttcctcctaagcccaaggacaccctgatgatttctagaactccagaggtaacatgtgtggtcgtggacgtcagccatgaggaccccgaagtaaaatttaattggtacgtggacggggtggaggtgcataacgccaagaccaaaccccgagaggagcagtataatagcacctacagagtcgtgagtgttttgactgtcctgcatcaagattggctcaacggaaaggagtacaagtgcaaggtaagtaataaagccctgccagcacccatagagaaaaccatcagcaaagcaaaagggcagccaagagagccacaggtttatacactccctccatctcgggacgagttgactaagaaccaggttagtctgacatgtctggtgaaaggcttttaccccagcgatatagctgtagagtgggaaagcaacgggcagcctgagaacaactataagaccactccccccgttctggatagcgacggtagtttctttctttattccaagctgactgtggataaatcccgatggcagcagggaaatgtcttttcctgttccgtcatgcacgaagctctccataatcactacactcagaaatctctgtccctgagtcccggtaaa

SEQ ID NO: 118 - DNA, 1344 bp, Artificial Sequence
  Heavy chain nucleic acid
caagttcaacttaaacagagtggtcctggcctggtacagccaagccagtccctgtccataacctgtacagtctcaggctttagccttactaactatggcgtccactgggttcggcagtctcctggtaagggacttgaatggctcggagctatttggaggggagggtccaccgactataatgcagcattcatgtcccggctttctattaccaaggataacagcaaaagtcaggtgttctttaagatgaactccctgcagcccgacgatacagccatctactactgtgccaaaaacctgtatggaccagctgccatggactattggggccaggggacctccgtcacagttagcagcgcttctactaagggcccctctgtgtttccactggcaccatcctctaagtcaaccagtgggggcactgcagctttgggctgtctggtcaaggattactttccagaacctgtcaccgtttcctggaattccggcgccttgacttctggtgtgcacaccttcccagctgtcctgcagtccagtggactgtattcactgagctccgtggtcacagtgccttcaagtagcctgggcacccagacctatatctgtaacgtaaaccacaagccttctaacaccaaggtggacaaaaaggtggaaccaaaaagctgtgacaaaactcatacatgcccaccttgtccagctccagagctgctgggtggaccatctgtgttcttgtttccccctaagcccaaagacaccctgatgatttcacggacccctgaggtgacatgtgtcgttgtggacgttagtcacgaggatcctgaagtaaaattcaattggtacgtcgacggcgtcgaagttcacaatgccaagacaaagccccgagaggaacagtacaatagtacataccgagtcgtgagcgtgttgacagttctccaccaggattggctcaatggcaaagagtacaaatgtaaggtatcaaataaagccctccccgctcccatagagaagacaatctcaaaggccaaagggcaacccagggagcctcaagtctacacccttcccccatctcgcgacgagctcaccaagaatcaggtatcattgacatgtctggtcaaaggattttatcctagcgatatcgccgtagagtgggagtctaatggccagcccgagaataattacaaaaccactcctcctgtgctggattctgatggatctttcttcctgtatagtaagttgacagtcgataagtcccggtggcaacagggtaatgttttcagctgttccgttatgcatgaggctctccacaaccattacacccaaaagagtctttctctttcccccggcaag

SEQ ID NO: 119 - DNA, 1335 bp, Artificial Sequence
  Heavy chain nucleic acid
cagattcagctggtgcagtccggtcctgagttgaaaaagccaggagaaactgtcaacatctcttgccgcgccagcggggacacattcaccaactacgccgtgaactgggtgaagcaggctcccggtaaggggctcaagtggatgggctggatagacacctacacaggagatcctacctacgcagacgacttgaagggcaggttcgccttctctcttgagacctccgccacaactgcttatcttcagatcaacaatttgaaaaacgaggataccgccacttacttctgtgctggggatacctggttcgcttactgggggcagggaactttggtcaccgtgagcgctgcttccaccaaggggcccagcgtcttccctctcgccccttctagcaagagcaccagtggtggaaccgctgcccttgggtgcctcgtgaaggattatttccctgaacctgtgactgtatcttggaatagcggtgcactcacctccggtgtgcacacttttccagctgtgctgcagtctagcgggctgtatagccttagctccgtggtgaccgtgccttcttcttctttgggtacccagacctacatctgcaacgtgaaccataagccatccaatactaaggtcgacaagaaagtggagcccaaatcctgcgataaaacccatacttgtcccccatgcccagctccagaactgctcggagggcccagtgtgtttcttttccccccaaagcctaaggacactctcatgatatcccgaaccccagaggtgacatgcgtcgttgtggatgtgtcacatgaagatccagaggtcaagttcaactggtatgtcgatggagtcgaggtccacaatgctaagacaaaaccaagagaggagcagtataacagtacctaccgagtggtctccgtgctgactgttcttcaccaagattggctcaatggtaaagagtacaaatgcaaagtatccaacaaggcccttcctgcaccaatcgaaaaaactatttccaaagcaaagggccagcccagggagccacaggtctacacactgcccccaagcagggatgaactcaccaagaaccaagtgtcacttacctgcctggttaagggattctacccctccgatattgcagtagaatgggagtctaacggtcagcctgagaataactacaagaccaccccccccgtgctcgatagcgatggttcattcttcctgtatagcaagctgaccgtcgataagagtaggtggcaacaagggaatgtgttcagttgctctgttatgcacgaagccctccacaaccattacacacaaaagtccctttccttgtctcccggtaag

SEQ ID NO: 120 - DNA, 1350 bp, Artificial Sequence
  Heavy chain nucleic acid
caggtaacacttaaggagagcggtccaggtatactccagcctagtcagacactgagccttacatgcagctttagtgggttcagcctgtccagttctggcatgtcagtaggctggatccgacaacctagcggaaagggcctggagtggctggctcatatctggtggtcagacgacaagtcctataacccagccctcaaatcccgtctgaccatctcaaaagacaccagtaataatcaggttttccttaaaatcgcctctgtggtcactgcagatacagcaacctactactgtgcacgcgagggctctttgggctatggactggactactggggtcagggtaccactctgactgtaagcagtgcttcaactaagggacctagcgtgtttccactggcacccagttctaaatctacttctgggggaactgctgctctgggatgcctggtgaaggattactttccagagcccgtcactgtgagttggaatagcggtgccttgacctccggagtgcacaccttcccagctgtgcttcaatcctcaggcttgtattctctttcctccgtggtgaccgtgccatcctcctctctcgggacccagacttacatatgtaatgtaaaccacaagccctctaacacaaaggtagataagaaggtcgagccaaagtcctgtgataaaacacatacatgtcccccttgtcccgctccagaactgcttggcggaccctctgtgttcctgttcccacctaagccaaaagacactcttatgatttccaggactccagaggttacatgcgttgtggtggacgtaagccatgaagaccctgaggtgaaattcaattggtatgtggacggtgtggaggtccataacgctaagactaagcccagggaggaacagtacaactcaacatatcgagtcgtctctgtcctgacagtgctccaccaggattggctgaacggaaaggaatacaaatgcaaggtgagtaataaagccctgccagctcccatcgaaaaaacaatcagcaaggctaagggtcagccccgcgaaccacaggtctacactctgccaccaagtcgggatgaattgactaagaatcaggtctccctgacctgtctggtgaagggtttttatccctccgacatcgcagtggagtgggagagcaacgggcagcctgagaataactataagacaactccacccgtcctcgattctgacgggtctttttttctgtacagcaaactgactgtggacaagtctcggtggcagcaaggtaacgtcttcagctgctccgttatgcacgaagccctccacaaccactacactcagaaatctctgtccttgagtcctggaaag

SEQ ID NO: 121 - DNA, 1341 bp, Artificial Sequence
  Heavy chain nucleic acid
gaagttaaactcctcgaatctggaggagggttggttcagccaggtggttctttgaagctgtcttgtgcagcctctgggtttgacttccgaagatattggatgagttgggtgcgtcaggcaccagggaaaggtcttgaatggatcgggcagattaaccccgacagttcaatgattaactatacaccttctttgaaagataaatttattatatcccgtgacaacgctaaaaacaccttgtatctgcagatgagtgaggtgagatccgaagacactgctctttactactgtgccacctatggacaccacgctacagactcctggggccagggtaccagtgtgacagtttccagtgccagcaccaagggcccatccgttttccccctggctccctcttctaaatctacttcaggtggtactgcagcacttggatgtttggtgaaggactacttccctgagccagtcaccgtgagttggaactcaggcgctcttacatccggtgtccacacatttccagccgtgctgcagagctcaggactctattctctcagctccgtcgtgacagtacccagcagctcacttggcactcagacctatatctgtaatgtcaaccataagccatctaacactaaagtcgacaagaaggtcgagcccaagagttgcgacaaaacacatacctgccccccatgtcccgcaccagaactcctgggtggtccaagcgtgtttttgtttccacctaaaccaaaggacaccctgatgattagccgtacaccagaggtgacttgcgtggtggtcgacgtgagtcacgaggatccagaggtgaagttcaactggtatgtggatggcgttgaagtacataacgccaaaaccaagcccagagaggaacaatacaatagcacctaccgagtcgtcagtgtgctgaccgtgcttcaccaagactggctcaatgggaaagagtacaagtgtaaggttagcaataaagctctgcctgccccaatcgagaagactatctctaaggcaaagggacagcctagggagccacaggtctatacactccccccctcacgggatgagttgactaagaatcaggtgtcactgacatgccttgttaaggggttttatccctctgacatcgccgtcgaatgggagagtaacggacagccagagaacaactacaagacaaccccaccagtcctggactctgacggctccttctttctgtattcaaagctgactgtggacaaatctagatggcagcagggcaacgtctttagttgtagtgtgatgcatgaggctctgcataaccattatacacagaaaagtctcagtcttagcccaggaaaa

SEQ ID NO: 122 - DNA, 1344 bp, Artificial Sequence
  Heavy chain nucleic acid
caagttcagttgcagcaacctggtgcagaattggtgagacccggtacatctgtcaagatgagctgcaaggccagcgggtacacctttactagttactggatgcattgggtgaaacagcgacccggacaaggattggagtggatcggtgatatctatccagggtccgacagcacaaactatcatgagaagtttaagagcaaggccaccctgacagtcgacacctcaagctccacagcttacatgcagctgtcttccttgactagcgaagattctgccgtgtactattgcgctcttgatagctctgggtatgggttcgcctattggggacagggtactctggtgacagtgtccgctgcctccaccaaaggtcccagtgtatttcccttggctccatctagcaagtctacatccggcggtacagccgcactggggtgtcttgtgaaggactacttccctgagcctgttacagtatcttggaattcaggcgctctgacctcaggagtccacacctttcccgctgtcttgcagtctagcggcctttatagcctgtcttccgtggtgactgttccctctagtagtctcggaacccagacctacatctgcaatgtgaatcacaagcccagcaacacaaaagtcgacaagaaggtggagcctaaatcttgcgacaagactcacacctgtcctccatgccctgctccagaactgctggggggaccttcagtgtttctcttccctccaaaaccaaaggacaccctcatgatctcccgcaccccagaggtcacctgtgtggtggtggacgtttcacatgaggatcctgaggtgaagttcaattggtatgtggacggtgtcgaagttcataatgccaagacaaagcctcgcgaagagcagtacaacagcacttacagggtggtgtccgtcctgactgtcttgcaccaggactggttgaacggcaaagaatacaaatgcaaggtgagtaacaaggcactgcctgcaccaattgagaagactatctctaaggccaaaggccagcctcgtgagcctcaagtgtataccctcccccctagcagagacgagttgacaaagaatcaggtcagcttgacatgcctggtgaagggattttatccctcagacattgccgtcgaatgggagagtaacggccagccagaaaataactacaagacaaccccccctgtcctggacagcgatggatcatttttcctgtactcaaagctgactgtagacaagtctcgatggcagcaaggtaacgtattctcctgcagcgtgatgcacgaggcactgcataatcactatacccagaagagcctgagcctgtcaccagggaag

SEQ ID NO: 123 - DNA, 1350 bp, Artificial Sequence
  Heavy chain nucleic acid
gaggtacagttggttgaatctggcggtggacttgtacaaccaggcgaatccttgaaactgagttgcgactctaatgactatgaattccctagtcacgatatgtcttgggttcggaagacccctgagaaacggcttgagcttgtggcagccatcaactcagacggtggaaatacatattaccccgacactatggagaggagatttattatcagccgcgataataccaaaaggacactttatctccagatgtcctctttgcgcagcgaggataccgctctgcattattgtgcccgcccacgaccaactatcggcacaacagccactgggtcttctatgtctggggcacagggcccaagaagcgcctctacaaagggcccttcagtgtttccccttgctccatcatctaaaagtactagtggaggcactgctgctctcggctgtctggtcaaggactactttccagagcctgtgaccgtgtcctggaatagtggggcacttacatctggtgtacacacctttcctgctgtactgcagagctctggcctgtattccctgagcagcgttgtcacagtcccatcaagtagcttgggaactcagacctacatatgcaatgtgaatcacaaaccatccaacaccaaggtggataagaaggtcgagcctaaaagttgtgacaaaacccatacatgcccaccttgtcctgctcctgagcttctgggcggcccaagtgttttccttttccctcccaaacccaaagacacactcatgatttcccgtacacccgaggttacttgtgtggttgttgatgtcagccacgaggatcctgaggtcaagttcaactggtatgtcgacggcgttgaagtccacaacgctaagaccaagcctcgggaggagcagtataattccacttacagggtggtttccgttttgaccgtgctgcaccaggattggctgaacggcaaagagtataaatgcaaggtaagcaataaagccttgcccgcacccatcgagaaaactattagcaaagccaagggtcagcccagagaacctcaagtttataccctccctccctcacgcgatgaactcactaagaatcaggtgtcactcacatgtctcgtgaagggtttttacccctccgatattgctgtggagtgggagtctaacggccagcccgagaacaattacaagaccaccccacctgtgcttgatagcgatggctcctttttcctctacagtaagctgacagtggacaaatctcggtggcagcagggcaacgttttctcctgttcagtaatgcatgaggcattgcataatcattatacccaaaagagcctctcactttccccagggaag

SEQ ID NO: 124 - DNA, 642 bp, Artificial Sequence
  Heavy chain nucleic acid
tctattgtgatgactcaaagtcctaaatttctcctcgtttccgctggagacagggtgataataacttgcaaagcttcacagagcgtgtccaatgatgtagtatggtaccagcaaaagccaggccagagcccaaaacttctgatctactacggttcacaccggaacaccggagtacctacaaggtttactggttccggctatgggactgacttcacattcactatttcaaccgtccaggctgaagacctggcagtctatttttgtcagcaggatttcttctcaccatggaccttcggtggcggtacaaaattggagataaagcgcactgttgccgccccttccgtctttatatttccaccctccgatgagcagcttaagtcagggacagcctctgtcgtgtgtctgctcaacaatttttacccccgtgaagccaaggtgcagtggaaagtggataacgccctgcagtctggcaattctcaggagtcagtgaccgaacaggattcaaaagactctacatatagcttgtcatccacccttactctgtccaaagcagattatgagaagcacaaagtttatgcttgtgaagttactcaccagggtctgagcagccccgtaacaaagagtttcaaccggggggagtgc

SEQ ID NO: 125 - DNA, 654 bp, Artificial Sequence
  Light chain nucleic acid
gacatagttttgacccagtctcctgctagtctcgccgtgtctctgggacaacgagcaactattagctgccgagccagcgagagcgttgacgaatacgggatttctttcatgaactggttccagcagaagccaggacagcccccacgtctgcttatttacagcgcctctaaccaagggagtggagtgccagccagatttacaggtagtggatctggaacagatttctccctcaacattctgcccatggaggaagacgacaccgctatgtatttctgtcagcagagcaaagaagtgccctggacctttgggggagggaccaaactcgaaattaagcgaactgtggctgctccctcagtctttatcttcccaccatctgatgagcaactgaagtctggcactgcatccgtggtgtgtctgctgaataatttttaccctcgcgaggcaaaagtgcagtggaaggtggataatgctttgcagtcaggaaattctcaggagagtgttaccgaacaagatagcaaggactccacatatagtttgtcctctacacttactctctccaaggcagactacgaaaaacacaaggtgtacgcttgtgaggtgacccaccagggcctgagctcaccagtgaccaaatccttcaaccgcggcgagtgc

SEQ ID NO: 126 - DNA, 642 bp, Artificial Sequence
  Light chain nucleic acid
agtattgtgatgactcagacccccaagtttcttttggtgagtgcaggtgacagagtgacaattacctgcaaagcttcacagagcgtcagcaacgatgtcgtctggtaccgacagaagccaggacagagccccaagctcctcatctactacgcctctaatcgatacatcggcgtgcccgacaggtttaccggttctggctatggtactgattttaccttcacaatttctactgttcaggccgaagatcttgccgtgtacttttgtcaacaggattactatagtccctggactttcggcgggggtactaagctggagattaagcgaacagtggccgcaccctccgtgtttatcttcccaccctctgacgagcaactcaagagtggaactgcctctgtcgtgtgcttgctgaacaacttttatcccagagaagccaaggtgcagtggaaggtggataatgccctgcagagcgggaactctcaggaatccgtcacagaacaggattcaaaggactcaacttattccctttcttcaaccctgacactctccaaggccgactacgagaagcataaagtgtacgcttgcgaggtaacccatcagggtctcagtagccccgtcactaagagcttcaacaggggcgaatgc

SEQ ID NO: 127 - DNA, 657 bp, Artificial Sequence
  Light chain nucleic acid
gatgtggttatgactcagtccccattgtccttgccagtctctctgggtgatcaggcatctatttcctgcaggtccagccagtcactggtgcattctaacgggaacacctaccttcactggtacttgcaaaagcccgggcagagccctaagctgctcatttataaggtttctaatagattcagcggtgtgcctgacagattttctggttctggatctggcaccgattttaccctgaagatcagtcgagtagaggccgaagacttgggagtgtacttttgctcccaatccacccatgtcccaccagatgtgcgatggcgtcatcaggctggcaaccaaaggactgtggctgcccctagtgtttttatattcccaccttcagacgagcagcttaaaagtggcaccgcctcagtggtttgtttgctgaataatttctatccaagggaagcaaaggttcaatggaaggtggacaacgctctgcaaagcggaaacagtcaggagagcgtgaccgaacaggatagcaaggacagtacttatagtttgagcagtactctcactttgtccaaagccgactacgagaagcacaaggtgtacgcctgtgaagtgacccatcaggggttgtcaagccctgtaactaaaagttttaatagaggcgagtgt

SEQ ID NO: 128 - DNA, 642 bp, Artificial Sequence
  Light chain nucleic acid
gatatccagatgacccagaccacttccagtctttctgctagtctcggcgatcgagtgaccatcagctgccgagcatctcaggacatttcaaattcactcaactggtatcaacagaaaccagatggcaccgtcaagctgctgatttattacaccagcagactccacagtggtgtgcccagcaggttcacaggttctgggagtgggactgattactccctgacaatttctaacttggaacaggaggatatcgccacttatttttgccagcagggcaacacccttccattgacattcggcgcaggaaccaagcttgagttgaagagaaccgtcgccgcaccctctgtatttatattcccaccatctgacgagcaactgaagtccggaacagccagtgtggtgtgcctccttaataatttttaccctcgtgaagccaaggtacagtggaaggttgataacgccctgcagagtggaaatagtcaggagtctgtaaccgagcaggattccaaggattccacatattcactgtccagtacattgacattgtcaaaggctgactacgaaaagcataaggtatacgcctgcgaagtgacacatcaggggctgtcttctcccgtgacaaagtcattcaatcgcggggagtgt

SEQ ID NO: 129 - DNA, 642 bp, Artificial Sequence
  Light chain nucleic acid
gatattcagatgaaccagtctccctccagtttgtccgcctccctcggtgatactattactattacatgccacgctaaccaaaatatctacgtgtggctgtcatggtatcaacagaaacctggcaacattcccaagcttctcatctacgaggcatcaaacctgcacactggcgtcccttccaggttctcaggctcaggttccggaaccggttttacactgaccataagtagcctgcagcctgaagacatcgcaacctactattgtcagcaaggccagagctacccctacaccttcggcggaggaacaaagttggagataaaacggactgtcgccgcaccatctgtcttcatcttcccccccagtgatgagcagcttaagagtgggactgcttctgtcgtctgtctgctcaataatttttatccaagagaggctaaggtacagtggaaagtggacaacgctctgcagtctggtaactcccaggagagtgtgaccgaacaggattctaaggactccacatactccctgagctctacacttacactcagtaaagcagactatgagaagcataaagtgtatgcatgtgaggtgacacaccagggtctgtcctctcctgtgaccaagtctttcaacagaggcgagtgt

SEQ ID NO: 130 - DNA, 660 bp, Artificial Sequence
  Light chain nucleic acid
gatatcgtcatgtcacagagcccatctagcttggctgtctcagtgggcgagaaagtgaccatgtcatgcaaaagctcccaatccttgttgtacagccgcaatcagaaaaatcgccttgcctggtaccagcagaaaccaggtcagtccccaaaactgctgatttattgggccagcacaagggagagtggtgtgcccgatagattcactgggtccgggtcaggcaccgatttcactctgaccatctcatctgttaaagcagaagatctggccgtctactattgccagcagtactatagttatcccagaactttcgggggcggcaccaacctggagattaagcgtaccgtggcagcaccatctgtctttattttccccccatcagacgagcagttgaaatcaggaaccgcctctgtcgtgtgcctgctgaacaatttttatccaagagaggctaaggtgcagtggaaggtcgataatgctcttcagagcggtaattctcaagaatccgtgactgaacaggattcaaaagatagcacatacagtctctccagcaccctgaccctcagcaaggccgactacgagaagcacaaagtgtatgcctgtgaagtgacacatcaaggtctctccagccctgtaactaagagttttaatagaggagaatgc

SEQ ID NO: 131 - DNA, 657 bp, Artificial Sequence
  Light chain nucleic acid
gatgtcctgatgacccagactccattgagccttcctgtctccttgggggatcaagcctccatttcatgcaggagctcccagagcatagtccacacagatggaaacacttacctggagtggtacttgcagaagccaggccagtctcccaaactgctcatctacaaagtgtctaatagattttccggtgtgcccgaccgtttttcaggtagcggctctggtactgatttcactctgaagatctcccgggtggaggctgaggatctgggcgtctattattgtttccagggctcccacgtgccatggaccttcggtggtggcaccaagttggagattaagaggacagtggccgcaccatccgtgttcatatttccccctagcgatgaacagttgaaaagcggtactgcctccgtggtttgtctgctgaataatttctaccctagagaagctaaagtgcaatggaaggttgacaatgccttgcagtcaggcaattctcaggagtctgtgaccgaacaggacagcaaggactccacctattctctgtcttcaacactgacacttagtaaggctgattacgaaaagcataaggtatacgcctgtgaggtgacacaccagggcctgagttcaccagtgaccaagtccttcaatcgcggggagtgt

SEQ ID NO: 132 - DNA, 654 bp, Artificial Sequence
  Light chain nucleic acid
gatattgtgcttacacagtccccagcttccctggccgtttcccttgggcagcgagccacaatctcctgccgcgcatctgagtccgttgatagctacggcaattcttttatgcactggtaccaacaaaaacctggtcagcctccaaagttgctgatttatcgcgcttccaaccttgagtccggtattcctgctcgctttagcggctctgggagtagaaccgacttcacactgaccattaacccagttgaggctgatgatgtggctacctattactgtcagcagtctaacgaagacccatttacatttgggtcaggcactaagctggagattaagcgtaccgtggccgccccaagcgttttcatcttccccccaagcgacgagcagctgaagtccggaactgcctccgtcgtttgtctcttgaacaatttttatcctagggaagcaaaagtacagtggaaggtagataacgctctgcaaagcggtaactcccaagaatccgtaaccgaacaagactcaaaggacagtacttattccctgtccagcaccctgactctttctaaagctgattatgaaaagcataaagtttatgcatgtgaggtgacccaccagggattgtcctccccagttactaaatcttttaaccggggggaatgc

SEQ ID NO: 133 - DNA, 642 bp, Artificial Sequence
  Light chain nucleic acid
aatatcgtcatgacccagactcctaaatttcttctggtctccgctggagaccgcgtcactataactcgtaaagccagccagtccgtgtctaatgacgttgtgtggtaccagcagaagcccgggcagtcccctaagctgctcatgtattacgcaagttatcgatacaccggcgtgcccgatcgattcaccggatccggttatggcaccgacttcacatttactatttctaacgttcaggccgaggacctcgccgtatacttttgtcaacaagactactccagcccttttacatttggaagcgggacaaaattggagatcaagaggactgttgccgcaccttccgtgtttatatttccaccttccgacgagcagctgaagtccgggaccgcaagcgtggtttgtctcctcaataatttctatcctcgagaggctaaggtccagtggaaggtggacaatgctctgcagagtggcaactctcaggagtcagttactgaacaagacagcaaggacagcacatattcactgtcatccactcttactctctccaaggctgactacgaaaagcataaggtttatgcctgcgaggttactcatcagggtctgtccagtccagtgaccaaatcttttaatagaggagagtgc

SEQ ID NO: 134 - DNA, 654 bp, Artificial Sequence
  Light chain nucleic acid
gatattgtcttgacccaatcacctgctagcctggctgtgagcttggggcagcgagcaaccatctcctgtagggcctctcagtccgtctccacaagtacttacaactacatgcattggtaccagcagaaacctggccaaccaccaaaattgctcattaagtatgctagcaaccttgagtcaggcgtccccgcaaggtttagcggatctggctcaggaaccgattttactctgaacattcaccccgtggaggaggaggacactgctacctactattgccagcatagttgggagatacccttcacctttggcagtggcacaaagctcgagattaagcgcactgttgccgcacccagcgtgtttatctttccacctagcgacgaacagcttaagtctggtactgctagtgtggtgtgtttgttgaataacttctacccccgtgaagccaaggttcagtggaaagtggataacgctctccagagcgggaacagtcaggagtctgttaccgaacaggactcaaaggacagcacttacagcctgagcagtactctgaccctgagtaaggccgattatgaaaagcacaaggtgtacgcctgtgaggtgacccaccagggtctgtcatcccctgtcaccaagtcttttaatcggggtgaatgc

SEQ ID NO: 135 - protein, 25 aa, Artificial Sequence [sequence omitted]

SEQ ID NO: 136 - protein, 17 aa, Artificial Sequence
  Heavy chain framework region
  misc_feature (10)..(10): Xaa can be any naturally occurring amino acid
  misc_feature (15)..(15): Xaa can be any naturally occurring amino acid
LGWVRQAPGXGLEWXGH

SEQ ID NO: 137 - protein, 38 aa, Artificial Sequence
  Heavy chain framework region
  misc_feature (10)..(10): Xaa can be any naturally occurring amino acid
  misc_feature (12)..(12): Xaa can be any naturally occurring amino acid
  misc_feature (14)..(14): Xaa can be any naturally occurring amino acid
  misc_feature (37)..(37): Xaa can be any naturally occurring amino acid
NYNEKFKGRXTXTXDTSTSTVYMELSSLRSEDTAVYXC

SEQ ID NO: 138 - protein, 11 aa, Artificial Sequence
  Heavy chain framework region
WGQGTLVTVSS

SEQ ID NO: 139 - protein, 26 aa, Artificial Sequence [sequence omitted]

SEQ ID NO: 140 - protein, 17 aa, Artificial Sequence
  Light chain framework region
  misc_feature (4)..(4): Xaa can be any naturally occurring amino acid
MNWXQQKPGQAPRLLIY

SEQ ID NO: 141 - protein, 36 aa, Artificial Sequence
  Light chain framework region
  misc_feature (33)..(33): Xaa can be any naturally occurring amino acid
  misc_feature (35)..(35): Xaa can be any naturally occurring amino acid
NQGSGIPARFSGSGSGTDFTLTISSLEPEDFAXYXC

SEQ ID NO: 142 - protein, 10 aa, Artificial Sequence
  Light chain framework region
FGGGTKLEIK

SEQ ID NO: 143 - protein, 119 aa, Artificial Sequence
  Heavy chain variable region
QVQLVQSGAEVKKPGASVKVSCKASDYTFTNYWLGWVRQAPGQGLEWMGHIYPGGGYTNYNEKFKGRVTMTRDTSTSTVYMELSSLRSEDTAVYYCARVRSGNDALDFWGQGTLVTVSS

SEQ ID NO: 144 - protein, 119 aa, Artificial Sequence
  Heavy chain variable region
QVQLVQSGAEVKKPGASVKVSCKASDYTFTNYWLGWVRQAPGQGLEWIGHIYPGGGYTNYNEKFKGRVTMTADTSTSTVYMELSSLRSEDTAVYFCARVRSGNDALDFWGQGTLVTVSS

SEQ ID NO: 145 - protein, 119 aa, Artificial Sequence
  Heavy chain variable region
QVQLVQSGAEVKKPGASVKVSCKASDYTFTNYWLGWVRQAPGQGLEWIGHIYPGGGYTNYNEKFKGRATLTADTSTSTVYMELSSLRSEDTAVYFCARVRSGNDALDFWGQGTLVTVSS

SEQ ID NO: 146 - protein, 119 aa, Artificial Sequence
  Heavy chain variable region
QVQLVQSGAEVKKPGASVKVSCKASDYTFTNYWLGWVRQAPGHGLEWIGHIYPGGGYTNYNEKFKGRATLTADTSTSTVYMELSSLRSEDTAVYFCARVRSGNDALDFWGQGTLVTVSS

SEQ ID NO: 147 - protein, 111 aa, Artificial Sequence
  Light chain variable region
EIVLTQSPATLSLSPGERATLSCRASESVDEYGISFMNWYQQKPGQAPRLLIYSASNQGSGIPARFSGSGSGTDFTLTISSLEPEDFAVYYCQQSKEVPWTFGGGTKLEIK

SEQ ID NO: 148 - protein, 111 aa, Artificial Sequence
  Light chain variable region
EIVLTQSPATLSLSPGERATLSCRASESVDEYGISFMNWYQQKPGQAPRLLIYSASNQGSGIPARFSGSGSGTDFTLTISSLEPEDFAVYFCQQSKEVPWTFGGGTKLEIK

SEQ ID NO: 149 - protein, 111 aa, Artificial Sequence
  Light chain variable region
EIVLTQSPATLSLSPGERATLSCRASESVDEYGISFMNWFQQKPGQAPRLLIYSASNQGSGIPARFSGSGSGTDFTLTISSLEPEDFAVYFCQQSKEVPWTFGGGTKLEIK

SEQ ID NO: 150 - protein, 111 aa, Artificial Sequence
  Light chain variable region
EIVLTQSPATLSLSPGERATLSCRASESVDEYGISFMNWFQQKPGQAPRLLIYSASNQGSGIPARFSGSGSGTDFTLTISSLEPEDFAMYFCQQSKEVPWTFGGGTKLEIK

SEQ ID NO: 151 - protein, 449 aa, Artificial Sequence
  Heavy chain
QVQLVQSGAEVKKPGASVKVSCKASDYTFTNYWLGWVRQAPGQGLEWMGHIYPGGGYTNYNEKFKGRVTMTRDTSTSTVYMELSSLRSEDTAVYYCARVRSGNDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 449 aa, Artificial Sequence
  Heavy chain
QVQLVQSGAEVKKPGASVKVSCKASDYTFTNYWLGWVRQAPGQGLEWIGHIYPGGGYTNYNEKFKGRVTMTADTSTSTVYMELSSLRSEDTAVYFCARVRSGNDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 449 aa, Artificial Sequence
  Heavy chain
QVQLVQSGAEVKKPGASVKVSCKASDYTFTNYWLGWVRQAPGQGLEWIGHIYPGGGYTNYNEKFKGRATLTADTSTSTVYMELSSLRSEDTAVYFCARVRSGNDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 449 aa, Artificial Sequence
  Heavy chain
QVQLVQSGAEVKKPGASVKVSCKASDYTFTNYWLGWVRQAPGHGLEWIGHIYPGGGYTNYNEKFKGRATLTADTSTSTVYMELSSLRSEDTAVYFCARVRSGNDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 218 aa, Artificial Sequence
  Light chain
EIVLTQSPATLSLSPGERATLSCRASESVDEYGISFMNWYQQKPGQAPRLLIYSASNQGSGIPARFSGSGSGTDFTLTISSLEPEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 218 aa, Artificial Sequence
  Light chain
EIVLTQSPATLSLSPGERATLSCRASESVDEYGISFMNWYQQKPGQAPRLLIYSASNQGSGIPARFSGSGSGTDFTLTISSLEPEDFAVYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 218 aa, Artificial Sequence
  Light chain
EIVLTQSPATLSLSPGERATLSCRASESVDEYGISFMNWFQQKPGQAPRLLIYSASNQGSGIPARFSGSGSGTDFTLTISSLEPEDFAVYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 218 aa, Artificial Sequence
  Light chain
EIVLTQSPATLSLSPGERATLSCRASESVDEYGISFMNWFQQKPGQAPRLLIYSASNQGSGIPARFSGSGSGTDFTLTISSLEPEDFAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - DNA, 1353 bp, Artificial Sequence
  Heavy chain nucleic acid
caagtgcagctggttcagagcggagctgaggtgaagaagcctggagctagcgtgaaggtgtcttgtaaagccagcgactacacctttaccaattactggctgggctgggtcagacaggcccctggccagggcctggaatggatgggccacatctaccccggcggaggctacacaaactacaacgagaagttcaagggcagagtgacaatgaccagagatacaagcaccagcacagtgtatatggaactgtctagcctgcggagcgaggacaccgccgtgtactactgcgccagagtgcggtccggcaacgacgccctggatttctggggccagggcaccctggtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 160 - DNA, 1353 bp, Artificial Sequence
  Heavy chain nucleic acid
caagtgcagctggtccagagcggcgccgaggtgaaaaagcctggagctagcgtgaaggtgtcctgcaaggccagcgactacaccttcaccaactattggctgggctgggtgcggcaggcccctggacagggcctggaatggatcggccacatctaccccggaggcggctacaccaattacaacgagaagttcaagggcagagtgaccatgaccgccgatacaagcacatctacagtgtacatggaactgagcagcctgagaagcgaggatacagctgtttacttctgtgccagagtgcggtccggcaacgacgccctggacttttggggccagggcaccctggtgaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 161 - DNA, 1353 bp, Artificial Sequence
  Heavy chain nucleic acid
caagtgcagctggtccagagcggcgccgaggtgaaaaagcctggcgcttctgtgaaggtgtcctgcaaggccagcgactacacatttaccaattattggctgggctgggtgcggcaggcccctggacagggcctggaatggatcggccacatctaccccggcggaggctacacaaactacaacgagaagttcaagggcagagccacactcaccgctgataccagcacaagcaccgtgtacatggaactgagcagcctgagaagcgaggacaccgccgtgtacttctgtgccagagtgcggtccggcaacgacgccctggatttctggggacagggcaccctggtgaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 162 - DNA, 1353 bp, Artificial Sequence
  Heavy chain nucleic acid
caagtgcagctggttcagagcggcgccgaggtgaagaagcctggcgctagcgtgaaggtgtcttgtaaagccagcgactacacatttaccaattactggctgggatgggtccggcaggcccctggccacggcctggaatggatcggccacatctaccccggcggaggatatacaaactacaacgagaagttcaagggcagagccaccctgaccgctgatacatctacaagcaccgtgtacatggaactgagcagcctgagaagcgaggataccgccgtgtacttctgcgccagagtgcggtccggcaacgacgccctggacttctggggccagggcaccctggtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 163 - DNA, 660 bp, Artificial Sequence
  Light chain nucleic acid
gagatcgtgctgacacagagccctgctacactctccctgtccccaggcgagcgggccacactgtcttgtagagccagcgagagcgtcgacgagtacggcattagcttcatgaactggtatcagcagaaacctggccaggcccctagactgctgatctacagcgcctccaaccagggcagcggcatccccgctagattcagcggcagcggatctggcaccgacttcaccctgaccatcagcagcctggaacccgaggattttgccgtgtactactgccagcaatctaaggaagtgccttggaccttcggcggaggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 164 - DNA, 660 bp, Artificial Sequence
  Light chain nucleic acid
gagatcgtgctgacccagagccccgccaccctgtccctgagccctggcgagcgggctacactgtcttgtagagccagcgagagcgtcgacgagtacggcatctcttttatgaactggtatcagcagaaacctggacaggcccctagactgctgatctacagcgcctccaaccagggcagcggcattcctgctagattcagcggcagcggctccggcacagatttcaccctcaccatcagcagcctggaacccgaggacttcgccgtgtacttctgccagcaatctaaggaagtgccatggacattcggcggaggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 165 - DNA, 660 bp, Artificial Sequence
  Light chain nucleic acid
gagatcgtgctgacccagtctccagccaccctctccctgagccccggcgagcgggccacactgtcttgtagagctagcgagagcgtcgacgagtacggcatctccttcatgaactggttccagcagaaacctggccaggcccctagactgctgatctacagcgccagcaaccagggctccggaattcctgctagattcagcggctctggcagcggcacagattttaccctgacaatcagcagcctggaacccgaggactttgccgtgtacttctgccagcaaagcaaggaagtgccttggaccttcggcggaggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 166 - DNA, 660 bp, Artificial Sequence
  Light chain nucleic acid
gagatcgtgctgacacagagccctgctacactctccctgagccccggcgagcgggccaccctgtcttgtagagccagcgagagcgtggacgagtacggcattagcttcatgaactggtttcagcagaaacctggccaggcccctagactgctgatctacagcgcctccaaccagggctctggcatccccgctagattcagcggatctggctccggcaccgacttcaccctgaccatcagcagcctggaaccagaggattttgccatgtacttctgccagcaaagcaaggaagtgccttggaccttcggcggaggcacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

(19) United States
(12) Patent Application Publication
(10) Pub. No.: US 2023/0257458 A1
(43) Pub. Date: Aug. 17, 2023

(54) ANTIBODY OF IL-11 AND USE THEREOF

(71) Applicant: SUNSHINE LAKE PHARMA CO., LTD., Dongguan, Guangdong (CN)

(72) Inventors: Jiatao ZHOU, Dongguan (CN); Cangsha CHEN, Dongguan (CN); Shujuan WANG, Dongguan (CN); Jie YAO, Dongguan (CN); Xiaofeng CHEN, Dongguan (CN); Wenjia LI, Dongguan (CN)

(73) Assignee: SUNSHINE LAKE PHARMA CO., LTD., Dongguan, Guangdong (CN)

(21) Appl. No.: 18/014,310

(22) PCT Filed: Aug. 12, 2021
(86) PCT No.: PCT/CN2021/112181

§ 371 Date: Jan. 3, 2023

(30) Foreign Application Priority Data
Aug. 13, 2020 (CN) 202010814358.3

(51) Int. Cl. C07K 16/24; C07K 14/54; G01N 33/68; C12N 15/85; A61P 11/00; C07K 14/715

(52) U.S. Cl. CPC C07K 16/244; C07K 14/5431; G01N 33/6869; C12N 15/85; A61P 11/00; C07K 14/7155; G01N 2333/5431; A61K 2039/505

(57) ABSTRACT

An antibody or an antigen-binding fragment can specifically recognize IL-11 thereof. The antibody contains CDR sequence(s) selected from at least one of the following or an amino acid sequence having at least 95% identity thereto: heavy chain variable region CDR sequences: SEQ ID NOs: 1-33 and light chain variable region CDR sequences: SEQ ID NOs: 34-66. The antibody can specifically target and bind to IL-11, so as to block binding of IL-11 and an IL-11 receptor.

### CROSS-REFERENCE TO RELATED APPLICATION

This application claims the priority and benefits of Chinese Patent Application No. 202010814358.3, filed with the State Intellectual Property Office of China on Aug. 13, 2020, which is incorporated herein by reference in its entirety.

### FIELD OF THE INVENTION

The present invention relates to the field of biotechnology, in particular, the present invention relates to an IL-11 antibody and use thereof, more particularly, the present invention relates to an antibody or an antigen-binding fragment thereof that can specifically recognize IL-11, a nucleic acid molecule, an expression vector, a recombinant cell, a pharmaceutical composition, pharmaceutical use, and a kit for detecting IL-11.

### BACKGROUND ART

Fibrosis is an important process and a critical part of wound healing. Excessive fibrosis is common in many rare and common diseases and is important in disease pathogenesis. Diseases characterized by excessive fibrosis include, but are not limited to: systemic sclerosis, scleroderma, hypertrophic cardiomyopathy, dilated cardiomyopathy (DCM), atrial fibrillation, ventricular fibrillation, myocarditis, cirrhosis, kidney disease, eye disease, asthma, cystic fibrosis, arthritis and idiopathic pulmonary fibrosis. Despite the high impact on human health, there is still an unmet medical need for treatments and diagnostics for fibrosis.

The true physiological role of interleukin 11 (IL-11) is unclear. IL-11 is most closely related to hematopoietic cell activation and thrombopoiesis, but has also been found to have pro- and anti-inflammatory, pro-angiogenic effects, and is important for neoplasia. TGFβ1 or tissue damage is known to induce the expression of IL-11. From the published literature, the role of IL-11 in fibrosis is unclear. IL-11 is thought to be important for pulmonary fibrosis and inflammation, and its expression levels correlate with collagen levels in the skin and respiratory system.

However, most studies suggest that IL-11 is anti-fibrotic in the heart and kidney, and anti-inflammatory in several tissues and chronic inflammatory diseases. In general, the molecular mode of action of IL-11 is thought to regulate the mRNA level of RNA expression through STAT3-mediated transcription.

Studies have demonstrated the administration of agents capable of inhibiting the action of interleukin 11 (IL-11) can treat, prevent or alleviate fibrosis in subjects in need of treatment. The agents capable of inhibiting the action of IL-11 can prevent or reduce the binding of IL-11 to the IL-11 receptor.

Currently the only drugs on the market for pulmonary fibrosis are pirfenidone and nintedanib, but these two drugs have poor efficacy, and drugs with a new mechanism are needed to solve this dilemma.

The development of agents with the effect of inhibiting IL-11 has become the direction of the development of drugs for the treatment and prevention of pulmonary fibrotic diseases.

### SUMMARY

The present invention aims to solve one of the technical problems in the related art at least to a certain extent. To this end, the present invention develops an Anti-IL-11 antibody with high neutralizing activity, which is expected to be used for the treatment or prevention of fibrosis-related diseases.

In the first aspect of the present invention, the present invention provides an antibody or an antigen-binding fragment thereof that can specifically recognize IL-11. According to the embodiments of the present invention, the antibody comprises CDRs selected from at least one of the following or an amino acid sequence having at least 95% identity thereto: a heavy chain variable region comprising CDRs having the amino acid sequence of SEQ ID NO: 1-33, a light chain variable region comprising CDRs having the amino acid sequence of SEQ ID NO: 34-66.

(SEQ ID NO: 1)
GYTFTTNG.
(SEQ ID NO: 2)
INTNTGEP.
(SEQ ID NO: 3)
AREGAFGLDY.
(SEQ ID NO: 4)
DYTFTNYW.
(SEQ ID NO: 5)
IYPGGGYT.
(SEQ ID NO: 6)
ARVRSGNDALDF.
(SEQ ID NO: 7)
GYTFTNYG.
(SEQ ID NO: 8)
INTNTGEP.
(SEQ ID NO: 9)
SREGIYGMDS.
(SEQ ID NO: 10)
GFSLTSYG.
(SEQ ID NO: 11)
IWAGGRT.
(SEQ ID NO: 12)
AREGGYDYDGDLWT.
(SEQ ID NO: 13)
GFSLTSYG.
(SEQ ID NO: 14)
IWSGGIT.
(SEQ ID NO: 15)
ARNFDGYYYSIDY.
(SEQ ID NO: 16)
GFSLTNYG.
(SEQ ID NO: 17)
IWRGGST.
(SEQ ID NO: 18)
AKNLYGPAAMDY.
(SEQ ID NO: 19)
GDTFTNYA.
(SEQ ID NO: 20)
IDTYTGDP.
(SEQ ID NO: 21)
AGDTWFAY.
(SEQ ID NO: 22)
GFSLSSSGMS.
(SEQ ID NO: 23)
IWWSDDK.
(SEQ ID NO: 24)
AREGSLGYGLDY.
(SEQ ID NO: 25)
GFDFRRYW.
(SEQ ID NO: 26)
INPDSSMI.
(SEQ ID NO: 27)
ATYGHHATDS.
(SEQ ID NO: 28)
GYTFTSYW.
(SEQ ID NO: 29)
IYPGSDST.
(SEQ ID NO: 30)
ALDSSGYGFAY.
(SEQ ID NO: 31)
DYEFPSHD.
(SEQ ID NO: 32)
INSDGGNT.
(SEQ ID NO: 33)
ARPRPTIGTTATGSSM.
(SEQ ID NO: 34)
QSVSND.
(SEQ ID NO: 35)
YGS.
(SEQ ID NO: 36)
QQDFFSPWT.
(SEQ ID NO: 37)
ESVDEYGISF.
(SEQ ID NO: 38)
SAS.
(SEQ ID NO: 39)
QQSKEVPWT.
(SEQ ID NO: 40)
QSVSND.
(SEQ ID NO: 41)
YAS.
(SEQ ID NO: 42)
QQDYYSPWT.
(SEQ ID NO: 43)
QSLVHSNGNTY.
(SEQ ID NO: 44)
KVS.
(SEQ ID NO: 45)
SQSTHVPPD.
(SEQ ID NO: 46)
QDISNS.
(SEQ ID NO: 47)
YTS.
(SEQ ID NO: 48)
QQGNTLPLT.
(SEQ ID NO: 49)
QNIYVW.
(SEQ ID NO: 50)
EAS.
(SEQ ID NO: 51)
QQGQSYPYT.
(SEQ ID NO: 52)
QSLLYSRNQKNR.
(SEQ ID NO: 53)
WAS.
(SEQ ID NO: 54)
QQYYSYPRT.
(SEQ ID NO: 55)
QSIVHTDGNTY.
(SEQ ID NO: 56)
KVS.
(SEQ ID NO: 57)
FQGSHVPWT.
(SEQ ID NO: 58)
ESVDSYGNSF.
(SEQ ID NO: 59)
RAS.
(SEQ ID NO: 60)
QQSNEDPFT.
(SEQ ID NO: 61)
QSVSND.
(SEQ ID NO: 62)
YAS.
(SEQ ID NO: 63)
QQDYSSPFT.
(SEQ ID NO: 64)
QSVSTSTYNY.
(SEQ ID NO: 65)
YAS.
(SEQ ID NO: 66)
QHSWEIPFT.

The above-mentioned antibody according to the embodiments of the present invention can specifically target and bind to IL-11, and block the binding of IL-11 and an IL-11 receptor.

In another aspect, the present invention provides an antibody or an antigen-binding fragment thereof that can specifically recognize IL-11. According to the embodiments of the present invention, the antibody comprises CDRs selected from at least one of the following or an amino acid sequence having at least 95% identity thereto: a heavy chain variable region comprising CDRs having the amino acid sequence of SEQ ID NO: 1-33, a light chain variable region comprising CDRs having the amino acid sequence of SEQ ID NO: 34-66, the antibody or antigen-binding fragment thereof has humanization modifications. It should be noted that the “humanization modification” mentioned in this application refers to amino acid changes that can reduce the immunogenicity of the antibody or antigen-binding fragment thereof, including the mutation, insertion, deletion of amino acid, duplication of chemical group, and so on. The immunogenicity of the above-mentioned humanized antibody according to the embodiments of the present invention is reduced, and the antibody can specifically target and bind to IL-11, and block the binding of IL-11 and IL-11 receptor.

According to the embodiments of the present invention, the above-mentioned antibody or antigen-binding fragment may further include at least one of the following additional technical features:

According to the embodiments of the present invention, the antibody comprises:

a heavy chain variable region comprising CDR1, CDR2, CDR3 as shown in SEQ ID NO: 1, 2 and 3 or an amino acid sequence having at least 95% identity to SEQ ID NO: 1, 2 and 3, respectively; or

a heavy chain variable region comprising CDR1, CDR2, CDR3 as shown in SEQ ID NO: 4, 5 and 6 or an amino acid sequence having at least 95% identity to SEQ ID NO: 4, 5 and 6, respectively; or

a heavy chain variable region comprising CDR1, CDR2, CDR3 as shown in SEQ ID NO: 7, 8 and 9 or an amino acid sequence having at least 95% identity to SEQ ID NO: 7, 8 and 9, respectively; or

a heavy chain variable region comprising CDR1, CDR2, CDR3 as shown in SEQ ID NO: 10, 11 and 12 or an amino acid sequence having at least 95% identity to SEQ ID NO: 10, 11 and 12, respectively; or

a heavy chain variable region comprising CDR1, CDR2, CDR3 as shown in SEQ ID NO: 13, 14 and 15 or an amino acid sequence having at least 95% identity to SEQ ID NO: 13, 14 and 15, respectively; or

a heavy chain variable region comprising CDR1, CDR2, CDR3 as shown in SEQ ID NO: 16, 17 and 18 or an amino acid sequence having at least 95% identity to SEQ ID NO: 16, 17 and 18, respectively; or

a heavy chain variable region comprising CDR1, CDR2, CDR3 as shown in SEQ ID NO: 19, 20 and 21 or an amino acid sequence having at least 95% identity to SEQ ID NO: 19, 20 and 21, respectively; or

a heavy chain variable region comprising CDR1, CDR2, CDR3 as shown in SEQ ID NO: 22, 23 and 24 or an amino acid sequence having at least 95% identity to SEQ ID NO: 22, 23 and 24, respectively; or

a heavy chain variable region comprising CDR1, CDR2, CDR3 as shown in SEQ ID NO: 25, 26 and 27 or an amino acid sequence having at least 95% identity to SEQ ID NO: 25, 26 and 27, respectively; or

a heavy chain variable region comprising CDR1, CDR2, CDR3 as shown in SEQ ID NO: 28, 29 and 30 or an amino acid sequence having at least 95% identity to SEQ ID NO: 28, 29 and 30, respectively; or

a heavy chain variable region comprising CDR1, CDR2, CDR3 as shown in SEQ ID NO: 31, 32 and 33 or an amino acid sequence having at least 95% identity to SEQ ID NO: 31, 32 and 33.

According to the embodiments of the present invention, the antibody comprises: a light chain variable region comprising CDR1, CDR2, CDR3 as shown in SEQ ID NO: 34, 35 and 36 or an amino acid sequence having at least 95% identity to SEQ ID NO: 34, 35 and 36, respectively; or

a light chain variable region comprising CDR1, CDR2, CDR3 as shown in SEQ ID NO: 37, 38 and 39 or an amino acid sequence having at least 95% identity to SEQ ID NO: 37, 38 and 39, respectively; or

a light chain variable region comprising CDR1, CDR2, CDR3 as shown in SEQ ID NO: 40, 41 and 42 or an amino acid sequence having at least 95% identity to SEQ ID NO: 40, 41 and 42, respectively; or

a light chain variable region comprising CDR1, CDR2, CDR3 as shown in SEQ ID NO: 43, 44 and 45 or an amino acid sequence having at least 95% identity to SEQ ID NO: 43, 44 and 45, respectively; or

a light chain variable region comprising CDR1, CDR2, CDR3 as shown in SEQ ID NO: 46, 47 and 48 or an amino acid sequence having at least 95% identity to SEQ ID NO: 46, 47 and 48, respectively; or

a light chain variable region comprising CDR1, CDR2, CDR3 as shown in SEQ ID NO: 49, 50 and 51 or an amino acid sequence having at least 95% identity to SEQ ID NO: 49, 50 and 51, respectively; or

a light chain variable region comprising CDR1, CDR2, CDR3 as shown in SEQ ID NO: 52, 53 and 54 or an amino acid sequence having at least 95% identity to SEQ ID NO: 52, 53 and 54, respectively; or

a light chain variable region comprising CDR1, CDR2, CDR3 as shown in SEQ ID NO: 55, 56 and 57 or an amino acid sequence having at least 95% identity to SEQ ID NO: 55, 56 and 57, respectively; or

a light chain variable region comprising CDR1, CDR2, CDR3 as shown in SEQ ID NO: 58, 59 and 60 or an amino acid sequence having at least 95% identity to SEQ ID NO: 58, 59 and 60, respectively; or

a light chain variable region comprising CDR1, CDR2, CDR3 as shown in SEQ ID NO: 61, 62 and 63 or an amino acid sequence having at least 95% identity to SEQ ID NO: 61, 62 and 63, respectively; or

a light chain variable region comprising CDR1, CDR2, CDR3 as shown in SEQ ID NO: 64, 65 and 66 or an amino acid sequence having at least 95% identity to SEQ ID NO: 64, 65 and 66, respectively.

According to the embodiments of the present invention, the above-mentioned antibody or antigen-binding fragment may further include at least one of the following additional technical features:

According to the embodiments of the present invention, the antibody comprises:

a heavy chain variable region comprising CDR1, CDR2, CDR3 as shown in SEQ ID NO: 1, 2 and 3 or an amino acid sequence having at least 95% identity to SEQ ID NO: 1, 2 and 3, respectively; and

a light chain variable region comprising CDR1, CDR2, CDR3 as shown in SEQ ID NO: 34, 35 and 36 or an amino acid sequence having at least 95% identity to SEQ ID NO: 34, 35 and 36, respectively;

or, a heavy chain variable region comprising CDR1, CDR2, CDR3 as shown in SEQ ID NO: 4, 5 and 6 or an amino acid sequence having at least 95% identity to SEQ ID NO: 4, 5 and 6, respectively; and

a light chain variable region comprising CDR1, CDR2, CDR3 as shown in SEQ ID NO: 37, 38 and 39 or an amino acid sequence having at least 95% identity to SEQ ID NO: 37, 38 and 39, respectively;

or, a heavy chain variable region comprising CDR1, CDR2, CDR3 as shown in SEQ ID NO: 7, 8 and 9 or an amino acid sequence having at least 95% identity to SEQ ID NO: 7, 8 and 9, respectively; and

a light chain variable region comprising CDR1, CDR2, CDR3 as shown in SEQ ID NO: 40, 41 and 42 or an amino acid sequence having at least 95% identity to SEQ ID NO: 40, 41 and 42, respectively;

or, a heavy chain variable region comprising CDR1, CDR2, CDR3 as shown in SEQ ID NO: 10, 11 and 12 or an amino acid sequence having at least 95% identity to SEQ ID NO: 10, 11 and 12, respectively; and

a light chain variable region comprising CDR1, CDR2, CDR3 as shown in SEQ ID NO: 43, 44 and 45 or an amino acid sequence having at least 95% identity to SEQ ID NO: 43, 44 and 45, respectively;

or, a heavy chain variable region comprising CDR1, CDR2, CDR3 as shown in SEQ ID NO: 13, 14 and 15 or an amino acid sequence having at least 95% identity to SEQ ID NO: 13, 14 and 15, respectively; and

a light chain variable region comprising CDR1, CDR2, CDR3 as shown in SEQ ID NO: 46, 47 and 48 or an amino acid sequence having at least 95% identity to SEQ ID NO: 46, 47 and 48, respectively;

or, a heavy chain variable region comprising CDR1, CDR2, CDR3 as shown in SEQ ID NO: 16, 17 and 18 or an amino acid sequence having at least 95% identity to SEQ ID NO: 16, 17 and 18, respectively; and

a light chain variable region comprising CDR1, CDR2, CDR3 as shown in SEQ ID NO: 49, 50 and 51 or an amino acid sequence having at least 95% identity to SEQ ID NO: 49, 50 and 51, respectively;

or, a heavy chain variable region comprising CDR1, CDR2, CDR3 as shown in SEQ ID NO: 19, 20 and 21 or an amino acid sequence having at least 95% identity to SEQ ID NO: 19, 20 and 21, respectively; and

a light chain variable region comprising CDR1, CDR2, CDR3 as shown in SEQ ID NO: 52, 53 and 54 or an amino acid sequence having at least 95% identity to SEQ ID NO: 52, 53 and 54, respectively;

or, a heavy chain variable region comprising CDR1, CDR2, CDR3 as shown in SEQ ID NO: 22, 23 and 24 or an amino acid sequence having at least 95% identity to SEQ ID NO: 22, 23 and 24, respectively; and

a light chain variable region comprising CDR1, CDR2, CDR3 as shown in SEQ ID NO: 55, 56 and 57 or an amino acid sequence having at least 95% identity to SEQ ID NO: 55, 56 and 57, respectively;

or, a heavy chain variable region comprising CDR1, CDR2, CDR3 as shown in SEQ ID NO: 25, 26 and 27 or an amino acid sequence having at least 95% identity to SEQ ID NO: 25, 26 and 27, respectively; and

a light chain variable region comprising CDR1, CDR2, CDR3 as shown in SEQ ID NO: 58, 59 and 60 or an amino acid sequence having at least 95% identity to SEQ ID NO: 58, 59 and 60, respectively;

or, a heavy chain variable region comprising CDR1, CDR2, CDR3 as shown in SEQ ID NO: 28, 29 and 30 or an amino acid sequence having at least 95% identity to SEQ ID NO: 28, 29 and 30, respectively; and

a light chain variable region comprising CDR1, CDR2, CDR3 as shown in SEQ ID NO: 61, 62 and 63 or an amino acid sequence having at least 95% identity to SEQ ID NO: 61, 62 and 63, respectively;

or, a heavy chain variable region comprising CDR1, CDR2, CDR3 as shown in SEQ ID NO: 31, 32 and 33 or an amino acid sequence having at least 95% identity to SEQ ID NO: 31, 32 and 33, respectively; and

a light chain variable region comprising CDR1, CDR2, CDR3 as shown in SEQ ID NO: 64, 65 and 66 or an amino acid sequence having at least 95% identity to SEQ ID NO: 64, 65 and 66, respectively.

The CDRs of the present invention are defined with reference to the IMGT nomenclature system.

According to the embodiments of the present invention, the antibody or antigen-binding fragment thereof specifically recognizes IL-11.

According to the embodiments of the present invention, the antibody comprises at least one of a heavy chain framework region sequence and a light chain framework region sequence, and at least a part of at least one of the heavy chain framework region sequence and the light chain framework region sequence is derived from at least one of a murine antibody, a human antibody, a primate antibody, or a mutant thereof.

According to the embodiments of the present invention, the heavy chain framework region includes FL1, FL2, FL3 and FL4, and the FL1, FL2, FL3 and FL4 of heavy chain framework region have the amino acid sequences shown in SEQ ID NO: 135, 136, 137 and 138, respectively,

(SEQ ID NO: 135)
QVQLVQSGAEVKKPGASVKVSCKAS,
(SEQ ID NO: 136)
LGWVRQAPGX1GLEWX2GH,
(SEQ ID NO: 137)
NYNEKFKGRX3TX4TX5DTSTSTVYMELSSLRSEDTAVYX6C,
(SEQ ID NO: 138)
WGQGTLVTVSS,

wherein, X1 is Q or H, X2 is M or I, X3 is V or A, X4 is M or L, X5 is R or A, and X6 is Y or F.

According to the embodiments of the present invention, the light chain framework region includes FL1, FL2, FL3 and FL4, and the FL1, FL2, FL3 and FL4 of light chain framework region have the amino acid sequences shown in SEQ ID NO: 139, 140, 141 and 142, respectively,

(SEQ ID NO: 139)
EIVLTQSPATLSLSPGERATLSCRAS,
(SEQ ID NO: 140)
MNWX7QQKPGQAPRLLIY,
(SEQ ID NO: 141)
NQGSGIPARFSGSGSGTDFTLTISSLEPEDFAX8YX9C,
(SEQ ID NO: 142)
FGGGTKLEIK,

wherein, X7 is Y or F, X8 is V or M, and X9 is Y or F.

According to the embodiments of the present invention, the humanized antibody proposed by the present invention has a heavy chain variable region with the amino acid sequence shown in any one of SEQ ID NO: 143-146.

(SEQ ID NO: 143)
QVQLVQSGAEVKKPGASVKVSCKASDYTFTNYWLGWVRQAPGQGLEWM
GHIYPGGGYTNYNEKFKGRVTMTRDTSTSTVYMELSSLRSEDTAVYYCAR
VRSGNDALDFWGQGTLVTVSS.
(SEQ ID NO: 144)
QVQLVQSGAEVKKPGASVKVSCKASDYTFTNYWLGWVRQAPGQGLEWIG
HIYPGGGYTNYNEKFKGRVTMTADTSTSTVYMELSSLRSEDTAVYFCARV
RSGNDALDFWGQGTLVTVSS.
(SEQ ID NO: 145)
QVQLVQSGAEVKKPGASVKVSCKASDYTFTNYWLGWVRQAPGQGLEWIG
HIYPGGGYTNYNEKFKGRATLTADTSTSTVYMELSSLRSEDTAVYFCARV
RSGNDALDFWGQGTLVTVSS.
(SEQ ID NO: 146)
QVQLVQSGAEVKKPGASVKVSCKASDYTFTNYWLGWVRQAPGHGLEWIG
HIYPGGGYTNYNEKFKGRATLTADTSTSTVYMELSSLRSEDTAVYFCARV
RSGNDALDFWGQGTLVTVSS.

According to the embodiments of the present invention, the humanized antibody proposed by the present invention has a light chain variable region with the amino acid sequence shown in any one of SEQ ID NO: 147-150.

(SEQ ID NO: 147)
EIVLTQSPATLSLSPGERATLSCRASESVDEYGISFMNWYQQKPGQAPRL
LIYSASNQGSGIPARFSGSGSGTDFTLTISSLEPEDFAVYYCQQSKEVPW
TFGGGTKLEIK.
(SEQ ID NO: 48)
EIVLTQSPATLSLSPGERATLSCRASESVDEYGISFMNWYQQKPGQAPRL
LIYSASNQGSGIPARFSGSGSGTDFTLTISSLEPEDFAVYFCQQSKEVPW
TFGGGTKLEIK.
(SEQ ID NO: 149)
EIVLTQSPATLSLSPGERATLSCRASESVDEYGISFMNWFQQKPGQAPRL
LIYSASNQGSGIPARFSGSGSGTDFTLTISSLEPEDFAVYFCQQSKEVPW
TFGGGTKLEIK.
(SEQ ID NO: 150)
EIVLTQSPATLSLSPGERATLSCRASESVDEYGISFMNWFQQKPGQAPRL
LIYSASNQGSGIPARFSGSGSGTDFTLTISSLEPEDFAMYFCQQSKEVPW
TFGGGTKLEIK.

According to the embodiments of the present invention, the heavy chain variable region has the amino acid sequence shown in any one of SEQ ID NO: 67-77.

(SEQ ID NO: 67)
QIQLVQSGPELKKPGETVKISCKASGYTFTTNGINWVKQAAGKGLKWMGW
INTNTGEPTNAEELKGRFAFSLETSASTAYLQINNLKNEDAAIYFCAREG
AFGLDYWGQGTSVTVSS.
(SEQ ID NO: 68)
QVQLQQSGAELVRPGTSAKISCKASDYTFTNYWLGWLKQRPGHGLEWIGH
IYPGGGYTNYNEKFKGKATLTADTSSSTAYMQLSSLTSEDSAVYFCARVR
SGNDALDFWGQGTSVTVSS.
(SEQ ID NO: 69)
QIQLVQSGPELKKPGETVKISCKASGYTFTNYGMNWVKQAPGKGLKWMG
WINTNTGEPTYAEEIKGRFAFSLETSASTAYLQINNLKNEDTATYFCSRE
GIYGMDSWGQGTSVTVSS.
(SEQ ID NO: 70)
QVQLKESGPGLVAPSQSLSITCTVSGFSLTSYGVHWVRQPPGKGLEWLGI
IWAGGRTDYNSALVSRLNISKDNSKSQVFLKMHSLQTDDTAMYYCAREGG
YDYDGDLWTTGVKEPQSPSPQ.
(SEQ ID NO: 71)
QVQLKQSGPGLVLPSQSLSITCTVSGFSLTSYGVHWFRQSPGTGLEWLGV
IWSGGITDYNATFI.PRLSISKDNSKSQVFFKMNSLQADDTAIYYCARNF
DGYYYSIDYWGQGTSVTVSS.
(SEQ ID NO: 72)
QVQLKQSGPGLVQPSQSLSITCTVSGFSLTNYGVHWVRQSPGKGLEWLGA
IWRGGSTDYNAAFMSRLSITKDNSKSQVFFKMNSLQPDDTAIYYCAKNLY
GPAAMDYWGQGTSVTVSS.
(SEQ ID NO: 73)
QIQLVQSGPELKKPGETVNISCRASGDTFTNYAVNWVKQAPGKGLKWMGW
IDTYTGDPTYADDLKGRFAFSLETSATTAYLQINNLKNEDTATYFCAGDT
WFAYWGQGTLVTVSA.
(SEQ ID NO: 74)
QVTLKESGPGILQPSQTLSLTCSFSGFSLSSSGMSVGWIRQPSGKGLEWL
AHIWWSDDKSYNPALKSRLTISKDTSNNQVFLKIASVVTADTATYYCARE
GSLGYGLDYWGQGTTLTVSS.
(SEQ ID NO: 75)
EVKLLESGGGLVQPGGSLKLSCAASGFDFRRYWMSWVRQAPGKGLEWIGQ
INPDSSMINYTPSLKDKFIISRDNAKNTLYLQMSEVRSEDTALYYCATYG
HHATDSWGQGTSVTVSS.
(SEQ ID NO: 76)
QVQLQQPGAELVRPGTSVKMSCKASGYTFTSYWMHWVKQRPGQGLEWIG
DIYPGSDSTNYHEKFKSKATLTVDTSSSTAYMQLSSLTSEDSAVYYCALD
SSGYGFAYWGQGTLVTVSA.
(SEQ ID NO: 77)
EVQLVESGGGLVQPGESLKLSCDSNDYEFPSHDMSWVRKTPEKRLELVAA
INSDGGNTYYPDTMERRFIISRDNTKRTLYLQMSSLRSEDTALHYCARPR
PTIGTTATGSSMSGAQGPRS.

According to the embodiments of the present invention, the light chain variable region has the amino acid sequence shown in any one of SEQ ID NO: 78-88.

(SEQ ID NO: 78)
SIVMTQSPKFLLVSAGDRVIITCKASQSVSNDVVWYQQKPGQSPKLLIYY
GSHRNTGVPTRFTGSGYGTDFTFTISTVQAEDLAVYFCQQDFFSPWTFGG
GTKLEIK.
(SEQ ID NO: 79)
DIVLTQSPASLAVSLGQRATISCRASESVDEYGISFMNWFQQKPGQPPRL
LIYSASNQGSGVPARFTGSGSGTDFSLNILPMEEDDTAMYFCQQSKEVPW
TFGGGTKLEIK.
(SEQ ID NO: 80)
SIVMTQTPKFLLVSAGDRVTITCKASQSVSNDVVWYRQKPGQSPKLLIYY
ASNRYIGVPDRFTGSGYGTDFTFTISTVQAEDLAVYFCQQDYYSPWTFGG
GTKLEIK.
(SEQ ID NO: 81)
DVVMTQSPLSLPVSLGDQASISCRSSQSLVHSNGNTYLHWYLQKPGQSPK
LLIYKVSNRFSGVPDRFSGSGSGTDFTLKISRVEAEDLGVYFCSQSTHVP
PDVRWRHQAGNQ.
(SEQ ID NO: 82)
DIQMTQTTSSLSASLGDRVTISCRASQDISNSLNWYQQKPDGTVKLLIYY
TSRLHSGVPSRFTGSGSGTDYSLTISNLEQEDIATYFCQQGNTLPLTFGA
GTKLELK.
(SEQ ID NO: 83)
DIQMNQSPSSLSASLGDTITITCHANQNIYVWLSWYQQKPGNIPKLLIYE
ASNLHTGVPSRFSGSGSGTGFTLTISSLQPEDIATYYCQQGQSYPYTFGG
GTKLEIK.
(SEQ ID NO: 84)
DIVMSQSPSSLAVSVGEKVTMSCKSSQSLLYSRNQKNRLAWYQQKPGQSP
KLLIYWASTRESGVPDRFTGSGSGTDFTLTISSVKAEDLAVYYCQQYYSY
PRTFGGGTNLEIK.
(SEQ ID NO: 85)
DVLMTQTPLSLPVSLGDQASISCRSSQSIVHTDGNTYLEWYLQKPGQSPK
LLIYKVSNRFSGVPDRFSGSGSGTDFTLKISRVEAEDLGVYYCFQGSHVP
WTFGGGTKLEIK.
(SEQ ID NO: 86)
DIVLTQSPASLAVSLGQRATISCRASESVDSYGNSFMHWYQQKPGQPPKL
LIYRASNLESGIPARFSGSGSRTDFTLTINPVEADDVATYYCQQSNEDPF
TFGSGTKLEIK.
(SEQ ID NO: 87)
NIVMTQTPKFLLVSAGDRVTITRKASQSVSNDVVWYQQKPGQSPKLLMYY
ASYRYTGVPDRFTGSGYGTDFTFTISNVQAEDLAVYFCQQDYSSPFTFGS
GTKLEIK.
(SEQ ID NO: 88)
DIVLTQSPASLAVSLGQRATISCRASQSVSTSTYNYMHWYQQKPGQPPKL
LIKYASNLESGVPARFSGSGSGTDFTLNIHPVEEEDTATYYCQHSWEIPF
TFGSGTKLEIK.

According to the embodiments of the present invention, the heavy chain variable region has the amino acid sequence shown in SEQ ID NO: 67, and the light chain variable region has the amino acid sequence shown in SEQ ID NO: 78; or

the heavy chain variable region has the amino acid sequence shown in SEQ ID NO:68, and the light chain variable region has the amino acid sequence shown in SEQ ID NO:79; or

the heavy chain variable region has the amino acid sequence shown in SEQ ID NO:69, and the light chain variable region has the amino acid sequence shown in SEQ ID NO:80; or

the heavy chain variable region has the amino acid sequence shown in SEQ ID NO:70, and the light chain variable region has the amino acid sequence shown in SEQ ID NO:81; or

the heavy chain variable region has the amino acid sequence shown in SEQ ID NO:71, and the light chain variable region has the amino acid sequence shown in SEQ ID NO:82; or

the heavy chain variable region has the amino acid sequence shown in SEQ ID NO:72, and the light chain variable region has the amino acid sequence shown in SEQ ID NO:83; or

the heavy chain variable region has the amino acid sequence shown in SEQ ID NO:73, and the light chain variable region has the amino acid sequence shown in SEQ ID NO:84; or

the heavy chain variable region has the amino acid sequence shown in SEQ ID NO:74, and the light chain variable region has the amino acid sequence shown in SEQ ID NO:85; or

the heavy chain variable region has the amino acid sequence shown in SEQ ID NO:75, and the light chain variable region has the amino acid sequence shown in SEQ ID NO:86; or

the heavy chain variable region has the amino acid sequence shown in SEQ ID NO:76, and the light chain variable region has the amino acid sequence shown in SEQ ID NO:87; or

the heavy chain variable region has the amino acid sequence shown in SEQ ID NO:77, and the light chain variable region has the amino acid sequence shown in SEQ ID NO:88.

According to the embodiments of the present invention, the humanized antibody proposed by the present invention has a heavy chain variable region with the amino acid sequence shown in SEQ ID NO: 143, and a light chain variable region with the amino acid sequence shown in SEQ ID NO: 147; or

the humanized antibody proposed by the present invention has a heavy chain variable region with the amino acid sequence shown in SEQ ID NO: 144, and a light chain variable region with the amino acid sequence shown in SEQ ID NO: 148; or

the humanized antibody proposed by the present invention has a heavy chain variable region with the amino acid sequence shown in SEQ ID NO: 145, and a light chain variable region with the amino acid sequence shown in SEQ ID NO: 149; or

the humanized antibody proposed by the present invention has a heavy chain variable region with the amino acid sequence shown in SEQ ID NO: 146, and a light chain variable region with the amino acid sequence shown in SEQ ID NO: 150.

According to the embodiments of the present invention, the antibody comprises at least one of a heavy chain constant region and a light chain constant region, and at least a part of at least one of the heavy chain constant region and the light chain constant region is derived from at least one of a murine antibody, a human antibody, a primate antibody, or a mutant thereof.

According to the embodiments of the present invention, both the light chain constant region and the heavy chain constant region of the antibody are derived from a human IgG antibody or a mutant thereof. Furthermore, the immunogenicity of the antibody can be effectively reduced.

According to the embodiments of the present invention, the light chain constant region of the antibody is derived from the light chain constant region of human Kappa; the heavy chain constant region is derived from the heavy chain constant region of human IgG1.

According to the embodiments of the present invention, the full-length sequence of the antibody constant region is shown in SEQ ID NO: 89 or 90.

(SEQ ID NO: 89)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.
(SEQ ID NO: 90)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

Wherein, the full-length sequence of the antibody constant region shown in the above SEQ ID NO: 89 includes an IgG1 heavy chain constant region and a Fc region, wherein the sequence of the IgG1 heavy chain constant region is ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKV, the sequence of the Fc region is EPKSCDKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIE KTISKAKGQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSPGK; the full-length sequence of the antibody constant region shown in the above SEQ ID NO: 90 is an IgG light chain constant region.

According to the embodiments of the present invention, the heavy chain has the amino acid sequence shown in any one of SEQ ID NO: 91-101 and the light chain has the amino acid sequence shown in any one of SEQ ID NO: 102-112.

(SEQ ID NO: 91)
QIQLVQSGPELKKPGETVKISCKASGYTFTTNGINWVKQAAGKGLKWMGW
INTNTGEPTNAEELKGRFAFSLETSASTAYLQINNLKNEDAAIYFCAREGAFGLDYWGQG
TSVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK.
(SEQ ID NO: 92)
QVQLQQSGAELVRPGTSAKISCKASDYTFTNYWLGWLKQRPGHGLEWIGH
IYPGGGYTNYNEKFKGKATLTADTSSSTAYMQLSSLTSEDSAVYFCARVRSGNDALDFW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK.
(SEQ ID NO: 93)
QIQLVQSGPELKKPGETVKISCKASGYTFTNYGMNWVKQAPGKGLKWMG
WINTNTGEPTYAEEIKGRFAFSLETSASTAYLQINNLKNEDTATYFCSREGIYGMDSWGQ
GTSVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK.
(SEQ ID NO: 94)
QVQLKESGPGLVAPSQSLSITCTVSGFSLTSYGVHWVRQPPGKGLEWLGIIW
AGGRTDYNSALVSRLNISKDNSKSQVFLKMHSLQTDDTAMYYCAREGGYDYDGDLWT
TGVKEPQSPSPQASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK.
(SEQ ID NO: 95)
QVQLKQSGPGLVLPSQSLSITCTVSGFSLTSYGVHWFRQSPGTGLEWLGVIW
SGGITDYNATFI.PRLSISKDNSKSQVFFKMNSLQADDTAIYYCARNFDGYYYSIDYWGQ
GTSVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK.
(SEQ ID NO: 96)
QVQLKQSGPGLVQPSQSLSITCTVSGFSLTNYGVHWVRQSPGKGLEWLGAI
WRGGSTDYNAAFMSRLSITKDNSKSQVFFKMNSLQPDDTAIYYCAKNLYGPAAMDYW
GQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK.
(SEQ ID NO: 97)
QIQLVQSGPELKKPGETVNISCRASGDTFTNYAVNWVKQAPGKGLKWMGW
IDTYTGDPTYADDLKGRFAFSLETSATTAYLQINNLKNEDTATYFCAGDTWFAYWGQGTL
VTVSAASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK.
(SEQ ID NO: 98)
QVTLKESGPGILQPSQTLSLTCSFSGFSLSSSGMSVGWIRQPSGKGLEWLAHI
WWSDDKSYNPALKSRLTISKDTSNNQVFLKIASVVTADTATYYCAREGSLGYGLDYWG
QGTTLTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK.
(SEQ ID NO: 99)
EVKLLESGGGLVQPGGSLKLSCAASGFDFRRYWMSWVRQAPGKGLEWIGQ
INPDSSMINYTPSLKDKFIISRDNAKNTLYLQMSEVRSEDTALYYCATYGHHATDSWGQG
TSVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK.
(SEQ ID NO: 100)
QVQLQQPGAELVRPGTSVKMSCKASGYTFTSYWMHWVKQRPGQGLEWIG
DIYPGSDSTNYHEKFKSKATLTVDTSSSTAYMQLSSLTSEDSAVYYCALDSSGYGFAYWG
QGTLVTVSAASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCPPC
PAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK.
(SEQ ID NO: 101)
EVQLVESGGGLVQPGESLKLSCDSNDYEFPSHDMSWVRKTPEKRLELVAAI
NSDGGNTYYPDTMERRFIISRDNTKRTLYLQMSSLRSEDTALHYCARPRPTIGTTATGSS
MSGAQGPRSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK.
(SEQ ID NO: 102)
SIVMTQSPKFLLVSAGDRVIITCKASQSVSNDVVWYQQKPGQSPKLLIYYGS
HRNTGVPTRFTGSGYGTDFTFTISTVQAEDLAVYFCQQDFFSPWTFGGGTKLEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC.
(SEQ ID NO: 103)
DIVLTQSPASLAVSLGQRATISCRASESVDEYGISFMNWFQQKPGQPPRLLIY
SASNQGSGVPARFTGSGSGTDFSLNILPMEEDDTAMYFCQQSKEVPWTFGGGTKLEIKRT
VAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC.
(SEQ ID NO: 104)
SIVMTQTPKFLLVSAGDRVTITCKASQSVSNDVVWYRQKPGQSPKLLIYYAS
NRYIGVPDRFTGSGYGTDFTFTISTVQAEDLAVYFCQQDYYSPWTFGGGTKLEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC.
(SEQ ID NO: 105)
DVVMTQSPLSLPVSLGDQASISCRSSQSLVHSNGNTYLHWYLQKPGQSPKL
LIYKVSNRFSGVPDRFSGSGSGTDFTLKISRVEAEDLGVYFCSQSTHVPPDVRWRHQAGN
QRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC.
(SEQ ID NO: 106)
DIQMTQTTSSLSASLGDRVTISCRASQDISNSLNWYQQKPDGTVKLLIYYTS
RLHSGVPSRFTGSGSGTDYSLTISNLEQEDIATYFCQQGNTLPLTFGAGTKLELKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC.
(SEQ ID NO: 107)
DIQMNQSPSSLSASLGDTITITCHANQNIYVWLSWYQQKPGNIPKLLIYEAS
NLHTGVPSRFSGSGSGTGFTLTISSLQPEDIATYYCQQGQSYPYTFGGGTKLEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC.
(SEQ ID NO: 108)
DIVMSQSPSSLAVSVGEKVTMSCKSSQSLLYSRNQKNRLAWYQQKPGQSPK
LLIYWASTRESGVPDRFTGSGSGTDFTLTISSVKAEDLAVYYCQQYYSYPRTFGGGTNLEI
KRTVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC.
(SEQ ID NO: 109)
DVLMTQTPLSLPVSLGDQASISCRSSQSIVHTDGNTYLEWYLQKPGQSPKLL
IYKVSNRFSGVPDRFSGSGSGTDFTLKISRVEAEDLGVYYCFQGSHVPWTFGGGTKLEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC.
(SEQ ID NO: 110)
DIVLTQSPASLAVSLGQRATISCRASESVDSYGNSFMHWYQQKPGQPPKLLI
YRASNLESGIPARFSGSGSRTDFTLTINPVEADDVATYYCQQSNEDPFTFGSGTKLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC.
(SEQ ID NO: 111)
NIVMTQTPKFLLVSAGDRVTITRKASQSVSNDVVWYQQKPGQSPKLLMYY
ASYRYTGVPDRFTGSGYGTDFTFTISNVQAEDLAVYFCQQDYSSPFTFGSGTKLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC.
(SEQ ID NO: 112)
DIVLTQSPASLAVSLGQRATISCRASQSVSTSTYNYMHWYQQKPGQPPKLLI
KYASNLESGVPARFSGSGSGTDFTLNIHPVEEEDTATYYCQHSWEIPFTFGSGTKLEIKRT
VAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC.

Wherein, in this application, the antibody composed of the above SEQ ID NO: 91 and 102 is referred to as 3-6A4, the antibody composed of the above SEQ ID NO: 92 and 103 is referred to as 5-20E11-5F2, the antibody composed of the above SEQ ID NO: 93 and 104 is referred to as 3-5B2-2G4, the antibody composed of the above SEQ ID NO: 94 and 105 is referred to as 3-5B2-7A4, the antibody composed of the above SEQ ID NO: 95 and 106 is referred to as 3-2B5-3D2, the antibody composed of the above SEQ ID NO: 96 and SEQ ID NO:107 is referred to as 3-7C9-1A6, the antibody composed of the above SEQ ID NO: 97 and SEQ ID NO:108 is referred to as 3-9E1-3A2, the antibody composed of the above SEQ ID NO: 98 and SEQ ID NO:109 is referred to as 3-15D5-3A6, the antibody composed of the above SEQ ID NO: 99 and SEQ ID NO:110 is referred to as 3-15D5-6A1, the antibody composed of the above SEQ ID NO: 100 and SEQ ID NO:111 is referred to as 5-20E11-1A2, the antibody composed of the above SEQ ID NO: 101 and SEQ ID NO:112 is referred to as 5-21G2-1D3.

According to the embodiments of the present invention, the humanized antibody proposed by the present invention has a heavy chain with the amino acid sequence shown in any one of SEQ ID NO: 151-154 and a light chain with the amino acid sequence shown in any one of SEQ ID NO: 155-158.

(SEQ ID NO: 151)
VQLVQSGAEVKKPGASVKVSCKASDYTFTNYWLGWVRQAPGQGLEWM
GHIYPGGGYTNYNEKFKGRVTMTRDTSTSTVYMELSSLRSEDTAVYY
CARVRSGNDALDFWGQGTLVTVSSASTKGPSVFPLAPSSKSTSGGTA
ALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKAL
PAPIEKTISKAKGQPREPQVYTLPPSRDELTKNQVSLTCLVKGFYPS
DIAVEWESNGQPENNYKTTPPVLDSDGSFFLYSKLTVDKSRWQQGNV
FSCSVMHEALHNHYTQKSLSLSPGK.
(SEQ ID NO: 152)
QVQLVQSGAEVKKPGASVKVSCKASDYTFTNYWLGWVRQAPGQGLEW
IGHIYPGGGYTNYNEKFKGRVTMTADTSTSTVYMELSSLRSEDTAVY
FCARVRSGNDALDFWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK.
(SEQ ID NO: 153)
QVQLVQSGAEVKKPGASVKVSCKASDYTFTNYWLGWVRQAPGQGLEW
IGHIYPGGGYTNYNEKFKGRATLTADTSTSTVYMELSSLRSEDTAVY
FCARVRSGNDALDFWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK.
(SEQ ID NO: 154)
QVQLVQSGAEVKKPGASVKVSCKASDYTFTNYWLGWVRQAPGHGLEW
IGHIYPGGGYTNYNEKFKGRATLTADTSTSTVYMELSSLRSEDTAVY
FCARVRSGNDALDFWGQGTLVTVSSASTKGPSVFPLAPSSKSTSGGT
AALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSRDELTKNQVSLTCLVKGFYP
SDIAVEWESNGQPENNYKTTPPVLDSDGSFFLYSKLTVDKSRWQQGN
VFSCSVMHEALHNHYTQKSLSLSPGK.
(SEQ ID NO: 155)
EIVLTQSPATLSLSPGERATLSCRASESVDEYGISFMNWYQQKPGQA
PRLLIYSASNQGSGIPARFSGSGSGTDFTLTISSLEPEDFAVYYCQQ
SKEVPWTFGGGTKLEIKRTVAAPSVFIFPPSDEQLKSGTASVVCLLN
NFYPREAKVQWKVDNALQSGNSQESVTEQDSKDSTYSLSSTLTLSKA
DYEKHKVYACEVTHQGLSSPVTKSFNRGEC.
(SEQ ID NO: 156)
EIVLTQSPATLSLSPGERATLSCRASESVDEYGISFMNWYQQKPGQA
PRLLIYSASNQGSGIPARFSGSGSGTDFTLTISSLEPEDFAVYFCQQ
SKEVPWTFGGGTKLEIKRTVAAPSVFIFPPSDEQLKSGTASVVCLLN
NFYPREAKVQWKVDNALQSGNSQESVTEQDSKDSTYSLSSTLTLSKA
DYEKHKVYACEVTHQGLSSPVTKSFNRGEC.
(SEQ ID NO: 157)
EIVLTQSPATLSLSPGERATLSCRASESVDEYGISFMNWFQQKPGQA
PRLLIYSASNQGSGIPARFSGSGSGTDFTLTISSLEPEDFAVYFCQQ
SKEVPWTFGGGTKLEIKRTVAAPSVFIFPPSDEQLKSGTASVVCLLN
NFYPREAKVQWKVDNALQSGNSQESVTEQDSKDSTYSLSSTLTLSKA
DYEKHKVYACEVTHQGLSSPVTKSFNRGEC.
(SEQ ID NO: 158)
EIVLTQSPATLSLSPGERATLSCRASESVDEYGISFMNWFQQKPGQA
PRLLIYSASNQGSGIPARFSGSGSGTDFTLTISSLEPEDFAMYFCQQ
SKEVPWTFGGGTKLEIKRTVAAPSVFIFPPSDEQLKSGTASVVCLLN
NFYPREAKVQWKVDNALQSGNSQESVTEQDSKDSTYSLSSTLTLSKA
DYEKHKVYACEVTHQGLSSPVTKSFNRGEC.

According to the embodiments of the present invention, the antibody is a single-chain antibody, a multimeric antibody, a CDR-grafted antibody or a small molecule antibody.

According to the embodiments of the present invention, the single-chain antibody comprises a heavy chain variable region with the amino acid sequence shown in any one of SEQ ID NO: 67-77 and a light chain variable region with the amino acid sequence shown in any one of SEQ ID NO: 78-88, wherein the C-terminus of the heavy chain variable region is connected to the N-terminus of the light chain variable region through a connecting peptide linker, or the C-terminus of the light chain variable region is connected to the N-terminus of the heavy chain variable region through a connecting peptide linker. It should be noted that the “connecting peptide linker” of the single-chain antibody described in this application is a connecting peptide used to connect the heavy chain variable region and light chain variable region of the antibody, which can be a commonly used connecting peptide linker for preparing single-chain antibodies, and can also be a connecting peptide Linker modified by scientific researchers.

According to the embodiments of the present invention, the small molecule antibody comprises at least one of Fab antibody, Fv antibody, single domain antibody and minimum recognition unit.

In the second aspect of the present invention, the present invention provides a nucleic acid molecule. According to the embodiments of the present invention, the nucleic acid molecule encodes the aforementioned antibody or antigen-binding fragment thereof. The antibody or antigen-binding fragment encoded by the nucleic acid molecule according to the embodiments of the present invention can specifically target and bind to IL-11, and block the binding of IL-11 and IL-11 receptor.

According to the embodiments of the present invention, the above-mentioned nucleic acid molecule may further include at least one of the following additional technical features:

According to the embodiments of the present invention, the nucleic acid molecule is DNA.

According to the embodiments of the present invention, the nucleic acid molecule has the nucleotide sequence shown in any one of SEQ ID NO: 113-123 or has the nucleotide sequence shown in any one of SEQ ID NO: 124-134.

(SEQ ID NO: 113)
CAGATTCAATTGGTACAAAGCGGTCCCGAGCTCAAGAAACCTGGGGAGACTGTGAAAATTTCCTGTAAGGCCTCTGCCT
ACACCTTCACCACTAACGGTATCAACTGGGTGAAGCAGGCTGCAGGAAAGGGGCTCAAGTGGATGGGATGGATCAACAC
TAATACTGGAGAACCCACCAATGCTGAGGAACTTAAAGGCAGATTCGCCTTCTCTCTTGAGACTTCAGCCAGCACCGCC
TACCTCCAAATTAACAATCTGAAAAATGAGGATGCCGCTATCTATTTTTGTGCCCGAGAGGGTGCATTCGGTCTTGATT
ACTGGGGTCAGGGCACCTCTGTGACAGTGTCTAGCGCCTCAACTAAGGGGCCCAGTGTTTTCCCCCTTGCCCCTTCCTC
AAAATCAACTAGCGGAGGCACAGCCGCTCTTGGCTGTCTCGTGAAAGATTACTTCCCCGAGCCAGTGACAGTCTCTTGG
AACAGCGGGGCACTCACCTCCGGCGTTCACACTTTTCCCGCAGTCCTCCAGTCTAGCGGATTGTATAGCCTGTCTTCTG
TTGTCACAGTGCCATCCTCTAGCCTTGGCACCCAGACCTACATTTGTAATGTGAATCATAAGCCTTCAAACACAAAAGT
CGATAAAAAAGTGGAGCCAAAGAGCTGTGACAAGACTCACACTTGCCCTCCATGCCCTGCCCCAGAACTCCTTGGCGGA
CCTAGCGTCTTCCTGTTTCCTCCTAAGCCAAAGGACACCCTGATGATCTCACGCACTCCTGAAGTGACCTGCGTTGTGG
TAGACGTGAGCCACGAGGATCCAGAGGTGAAGTTCAACTGGTATGTGGATGGAGTCGAGGTGCATAATGCCAAAACAAA
ACCTAGGGAGGAGCAGTACAACTCTACCTACCGGGTGGTGTCTGTTTTGACCGTTCTGCACCAAGACTGGTTGAATGGC
AAGGAGTACAAGTGCAAGGTGTCAAACAAAGCACTCCCAGCCCCTATAGAGAAGACCATCAGCAAAGCTAAGGGGCAGC
CAAGAGAACCACAGGTTTACACCCTGCCCCCCTCACGGGATGAACTGACAAAGAACCAGGTGTCTCTGACTTGCCTCGT
GAAAGGATTCTACCCTTCAGATATCGCAGTGGAGTGGGAGTCTAACGGCCAACCCGAGAATAATTACAAGACAACTCCC
CCAGTCCTGGACAGTGATGGGTCTTTTTTCCTGTATTCTAAGCTCACTGTTGACAAAAGCCGCTGGCAGCAGGGTAATG
TATTCTCCTGCTCCGTCATGCACGAAGCCCTGCACAACCACTATACCCAGAAGTCTCTGAGCTTGTCACCCGGCAAG.
(SEQ ID NO: 114)
CAAGTACAGTTGCAGCAGTCTGGAGCTGAGCTGGTTAGACCTGGAACCAGTGCCAAGATCTCCTGCAAAGCTAGTGATT
ATACTTTCACAAACTACTGGCTCGGTTGGCTGAAACAAAGACCCGGGCACGGTCTGGAATGGATAGGCCACATTTACCC
AGGCGGCGGATACACCAACTATAACGAGAAGTTTAAGGGAAAGGCTACTCTGACAGCCGACACCTCATCCAGCACAGCC
TACATGCAGCTTAGTAGTTTGACCAGTGAGGATTCCGCAGTGTACTTCTGTGCACGTGTGAGGTCAGGGAATGACGCCC
TGGACTTCTGGGGCCAGGGTACTTCAGTGACAGTGTCCTCCGCTAGCACTAAGGGACCAAGCGTGTTTCCTCTGGCTCC
AAGCTCTAAATCAACCTCAGGCGGAACCGCCGCTTTGGGATGTCTTGTGAAGGATTACTTCCCTGAGCCTGTCACCGTC
TCCTGGAATAGCGGCGCCCTGACTTCCGGGGTGCATACATTCCCTGCCGTATTGCAGTCAAGCGGCCTGTATAGCCTTA
GCTCTGTTGTAACTGTGCCATCTTCTTCCCTTGGCACACAGACCTACATCTGTAACGTGAACCATAAGCCATCCAATAC
AAAAGTTGATAAGAAGGTGGAGCCAAAATCATGTGACAAAACTCATACATGCCCTCCTTGTCCAGCACCTGAGCTGTTG
GGCGGACCAAGTGTCTTTCTGTTCCCCCCAAAGCCAAAGGACACACTGATGATCAGTCGCACACCAGAAGTGACTTGCG
TTGTTGTCGATGTGAGCCACGAAGACCCTGAGGTTAAGTTTAACTGGTACGTGGATGGTGTGGAGGTGCACAATGCCAA
GACTAAGCCACGTGAAGAACAGTATAACTCCACCTATCGTGTGGTCTCTGTCCTGACAGTACTGCACCAGGACTGGCTC
AATGGAAAAGAGTACAAGTGCAAGGTTAGTAACAAGGCCTTGCCCGCTCCTATCGAGAAAACTATCAGTAAGGCAAAGG
GACAGCCAAGAGAACCACAGGTTTACACCCTGCCACCTAGCCGGGACGAATTGACTAAAAACCAAGTGAGTCTCACATG
CCTCGTCAAGGGATTCTACCCATCTGACATTGCCGTAGAGTGGGAGTCCAATGGGCAGCCCGAGAATAATTATAAGACT
ACTCCACCTGTCCTGGATTCTGATGGATCCTTCTTTTTGTATTCCAAGCTTACCGTGGACAAAAGCAGGTGGCAGCAGG
GCAATGTCTTTTCATGCAGCGTGATGCACGAAGCACTTCACAATCACTACACACAGAAGAGTCTGTCTCTCAGTCCTGG
TAAG.
(SEQ ID NO: 115)
CAAATTCAGCTGGTGCAGTCCGGCCCAGAACTTAAGAAACCCGGTGAAACCGTGAAGATATCCTGCAAAGCTTCAGGCT
ATACCTTTACCAACTATGGCATGAATTGGGTGAAGCAAGCTCCCGGGAAGGGCCTTAAATGGATGGGCTGGATCAACAC
CAACACAGGTGAGCCAACCTATGCCGAAGAAATCAAAGGACGCTTTGCTTTCTCTCTCGAAACTAGCGCAAGTACCGCT
TATTTGCAGATCAATAATCTGAAAAACGAGGACACCGCTACCTACTTTTGCTCAAGAGAGGGCATTTACGGCATGGATT
CTTGGGGCCAGGGAACATCTGTCACCGTCTCATCTGCATCTACCAAGGGCCCTAGTGTGTTCCCACTGGCCCCTTCATC
CAAGTCCACTTCTGGAGGAACAGCCGCCTTGGGCTGTCTCGTTAAGGACTACTTCCCCGAGCCTGTTACCGTGTCCTGG
AACAGTGGCGCCCTGACCTCCGGTGTTCACACTTTCCCCGCTGTGTTGCAGTCCTCTGGCCTTTATTCCCTTTCCTCTG
TAGTGACAGTGCCCAGTTCCAGTCTGGGTACACAGACCTACATTTGCAATGTCAACCATAAACCAAGTAATACCAAGGT
AGATAAAAAGGTAGAGCCTAAGTCATGCGATAAAACTCACACATGTCCACCATGTCCTGCTCCAGAACTCTTGGGCGGT
CCAAGTGTCTTTTTGTTCCCACCAAAACCTAAGGATACCCTCATGATCTCTAGGACCCCCGAGGTCACATGTGTGGTGG
TCGACGTTAGTCACGAGGATCCCGAGGTTAAATTTAACTGGTATGTCGATGGGGTTGAAGTCCACAATGCCAAAACCAA
ACCAAGAGAGGAACAGTACAATTCCACTTACCGTGTGGTTTCAGTGCTGACCGTCCTTCATCAGGACTGGCTCAACGGG
AAGGAGTACAAGTGCAAGGTCAGTAACAAGGCTCTTCCAGCCCCCATCGAAAAGACTATTTCCAAAGCCAAGGGACAAC
CAAGAGAGCCACAAGTGTACACTCTTCCCCCCAGCAGGGACGAACTCACAAAGAATCAGGTTTCTCTCACCTGTCTGGT
TAAGGGCTTTTACCCCAGCGACATCGCAGTTGAGTGGGAGAGCAATGGCCAGCCTGAGAATAACTATAAGACAACACCC
CCCGTGCTGGATTCTGACGGCAGCTTTTTCTTGTACTCTAAACTCACTGTAGACAAATCCAGGTGGCAGCAAGGCAACG
TGTTTTCCTGCTCTGTCATGCACGAGGCATTGCACAATCACTATACTCAGAAATCACTTAGCCTGTCCCCCGGTAAG.
(SEQ ID NO: 116)
CAAGTACAATTGAAAGAGAGTGGACCAGGACTTGTGGCCCCTAGCCAGTCTCTGTCAATAACCTGTACTGTCAGTGGAT
TTTCCCTTACCAGCTATGGGGTACACTGGGTTAGGCAGCCACCTGGTAAAGGACTTGAGTGGCTGGGCATTATTTGGGC
CGGAGGGCGGACAGACTATAATTCTGCACTGGTATCCAGACTCAATATCAGTAAGGACAACAGCAAAAGTCAAGTGTTT
CTGAAAATGCATTCACTTCAAACCGATGACACCGCTATGTACTACTGTGCTAGGGAAGGCGGTTATGACTATGATGGAG
ATCTCTGGACTACCGGAGTCAAGGAGCCTCAAAGCCCCAGTCCTCAGGCCTCCACAAAAGGGCCTAGTGTTTTCCCCCT
GGCCCCATCATCTAAGAGTACATCAGGAGGAACAGCAGCCCTTGGATGCCTGGTGAAAGACTACTTTCCAGAACCCGTT
ACCGTGTCCTGGAACTCAGGCGCACTTACCTCTGGCGTCCATACATTTCCCGCCGTATTGCAGAGCAGCGGACTGTACT
CATTGTCATCCGTGGTGACAGTGCCTAGTAGTTCTCTTGGTACACAGACCTACATATGTAACGTGAATCATAAACCCTC
AAATACTAAAGTCGACAAGAAGGTTGAGCCCAAGAGCTGTGATAAAACCCACACTTGTCCCCCATGCCCAGCACCTGAA
CTGCTCGGAGGCCCATCCGTCTTTCTCTTCCCTCCTAAGCCTAAGGATACACTTATGATTTCACGGACCCCTGAAGTGA
CATGTGTGGTCGTTGATGTGTCCCATGAGGACCCAGAAGTTAAATTTAACTGGTATGTCGATGGCGTGGAGGTGCACAA
CGCCAAGACAAAGCCCAGGGAAGAACAGTATAATTCAACATACCGCGTCGTTAGCGTGCTGACAGTTCTGCATCAGGAT
TGGCTTAACGGTAAAGAGTACAAGTGCAAGGTCTCCAACAAAGCTCTGCCTGCACCTATTGAAAAAACTATATCCAAGG
CTAAGGGCCAGCCTCGCGAGCCTCAGGTATATACACTGCCACCCAGCAGGGACGAACTGACAAAGAATCAGGTCAGTCT
GACTTGCCTCGTCAAGGGATTCTACCCTTCAGACATCGCCGTGGAGTGGGAGTCAAACGGTCAGCCCGAGAACAACTAC
AAGACAACACCCCCTGTACTGGACTCCGACGGCTCCTTTTTCCTGTATTCTAAGCTGACTGTCGATAAATCTCGCTGGC
AGCAGGGAAACGTCTTTTCCTGCTCTGTGATGCACGAGGCTCTGCACAATCACTATACTCAGAAGTCACTGTCCTTGTC
TCCCGGAAAG.
(SEQ ID NO: 117)
CAGGTACAATTGAAACAAAGTGGGCCAGGTCTTGTCCTTCCTTCCCAGTCTCTGAGTATTACCTGTACCGTCAGTGGAT
TTAGCCTTACAAGCTATGGAGTCCATTGGTTCAGGCAATCCCCAGGCACAGGCCTGGAATGGTTGGGGGTCATCTGGAG
TGGTGGTATCACCGATTACAATGCAACCTTTATCCCCCGTCTGTCCATATCAAAGGACAACTCTAAGTCCCAGGTGTTT
TTCAAAATGAACTCTCTCCAGGCAGATGACACAGCCATATATTACTGTGCTCGTAACTTCGACGGCTACTACTATTCCA
TAGATTATTGGGGCCAGGGCACTTCAGTCACCGTCAGTAGCGCCAGCACTAAGGGTCCTTCCGTTTTTCCCCTTGCTCC
TTCTTCAAAGAGCACAAGTGGGGGGACTGCAGCTTTGGGCTGCTTGGTGAAGGATTATTTTCCTGAGCCTGTCACAGTC
TCTTGGAATAGCGGGGCACTCACAAGCGGTGTTCATACCTTTCCAGCCGTGCTGCAGAGTTCTGGTCTGTATAGTCTCT
CCTCAGTGGTGACCGTACCTTCTTCCTCTCTGGGCACCCAGACATACATATGCAATGTTAATCACAAGCCCTCCAACAC
AAAAGTGGATAAAAAAGTTGAGCCTAAGAGTTGTGACAAAACTCACACATGTCCTCCTTGCCCAGCTCCCGAACTCCTT
GGAGGACCAAGTGTTTTTTTGTTTCCTCCTAAGCCCAAGGACACCCTGATGATTTCTAGAACTCCAGAGGTAACATGTG
TGGTCGTGGACGTCAGCCATGAGGACCCCGAAGTAAAATTTAATTGGTACGTGGACGGGGTGGAGGTGCATAACGCCAA
GACCAAACCCCGAGAGGAGCAGTATAATAGCACCTACAGAGTCGTGAGTGTTTTGACTGTCCTGCATCAAGATTGGCTC
AACGGAAAGGAGTACAAGTGCAAGGTAAGTAATAAAGCCCTGCCAGCACCCATAGAGAAAACCATCAGCAAAGCAAAAG
GGCAGCCAAGAGAGCCACAGGTTTATACACTCCCTCCATCTCGGGACGAGTTGACTAAGAACCAGGTTAGTCTGACATG
TCTGGTGAAAGGCTTTTACCCCAGCGATATAGCTGTAGAGTGGGAAAGCAACGGGCAGCCTGAGAACAACTATAAGACC
ACTCCCCCCGTTCTGGATAGCGACGGTAGTTTCTTTCTTTATTCCAAGCTGACTGTGGATAAATCCCGATGGCAGCAGG
GAAATGTCTTTTCCTGTTCCGTCATGCACGAAGCTCTCCATAATCACTACACTCAGAAATCTCTGTCCCTGAGTCCCGG
TAAA.
(SEQ ID NO: 118)
CAAGTTCAACTTAAACAGAGTGGTCCTGGCCTGGTACAGCCAAGCCAGTCCCTGTCCATAACCTGTACAGTCTCAGGCT
TTAGCCTTACTAACTATGGCGTCCACTGGGTTCGGCAGTCTCCTGGTAAGGGACTTGAATGGCTCGGAGCTATTTGGAG
GGGAGGGTCCACCGACTATAATGCAGCATTCATGTCCCGGCTTTCTATTACCAAGGATAACAGCAAAAGTCAGGTGTTC
TTTAAGATGAACTCCCTGCAGCCCGACGATACAGCCATCTACTACTGTGCCAAAAACCTGTATGGACCAGCTGCCATGG
ACTATTGGGGCCAGGGGACCTCCGTCACAGTTAGCAGCGCTTCTACTAAGGGCCCCTCTGTGTTTCCACTGGCACCATC
CTCTAAGTCAACCAGTGGGGGCACTGCAGCTTTGGGCTGTCTGGTCAAGGATTACTTTCCAGAACCTGTCACCGTTTCC
TGGAATTCCGGCGCCTTGACTTCTGGTGTGCACACCTTCCCAGCTGTCCTGCAGTCCAGTGGACTGTATTCACTGAGCT
CCGTGGTCACAGTGCCTTCAAGTAGCCTGGGCACCCAGACCTATATCTGTAACGTAAACCACAAGCCTTCTAACACCAA
GGTGGACAAAAAGGTGGAACCAAAAAGCTGTGACAAAACTCATACATGCCCACCTTGTCCAGCTCCAGAGCTGCTGGGT
GGACCATCTGTGTTCTTGTTTCCCCCTAAGCCCAAAGACACCCTGATGATTTCACGGACCCCTGAGGTGACATGTGTCG
TTGTGGACGTTAGTCACGAGGATCCTGAAGTAAAATTCAATTGGTACGTCGACGGCGTCGAAGTTCACAATGCCAAGAC
AAAGCCCCGAGAGGAACAGTACAATAGTACATACCGAGTCGTGAGCGTGTTGACAGTTCTCCACCAGGATTGGCTCAAT
GGCAAAGAGTACAAATGTAAGGTATCAAATAAAGCCCTCCCCGCTCCCATAGAGAAGACAATCTCAAAGGCCAAAGGGC
AACCCAGGGAGCCTCAAGTCTACACCCTTCCCCCATCTCGCGACGAGCTCACCAAGAATCAGGTATCATTGACATGTCT
GGTCAAAGGATTTTATCCTAGCGATATCGCCGTAGAGTGGGAGTCTAATGGCCAGCCCGAGAATAATTACAAAACCACT
CCTCCTGTGCTGGATTCTGATGGATCTTTCTTCCTGTATAGTAAGTTGACAGTCGATAAGTCCCGGTGGCAACAGGGTA
ATGTTTTCAGCTGTTCCGTTATGCATGAGGCTCTCCACAACCATTACACCCAAAAGAGTCTTTCTCTTTCCCCCGGCAA
G.
(SEQ ID NO: 119)
CAGATTCAGCTGGTGCAGTCCGGTCCTGAGTTGAAAAAGCCAGGAGAAACTGTCAACATCTCTTGCCGCGCCAGCGGGG
ACACATTCACCAACTACGCCGTGAACTGGGTGAAGCAGGCTCCCGGTAAGGGGCTCAAGTGGATGGGCTGGATAGACAC
CTACACAGGAGATCCTACCTACGCAGACGACTTGAAGGGCAGGTTCGCCTTCTCTCTTGAGACCTCCGCCACAACTGCT
TATCTTCAGATCAACAATTTGAAAAACGAGGATACCGCCACTTACTTCTGTGCTGGGGATACCTGGTTCGCTTACTGGG
GGCAGGGAACTTTGGTCACCGTGAGCGCTGCTTCCACCAAGGGGCCCAGCGTCTTCCCTCTCGCCCCTTCTAGCAAGAG
CACCAGTGGTGGAACCGCTGCCCTTGGGTGCCTCGTGAAGGATTATTTCCCTGAACCTGTGACTGTATCTTGGAATAGC
GGTGCACTCACCTCCGGTGTGCACACTTTTCCAGCTGTGCTGCAGTCTAGCGGGCTGTATAGCCTTAGCTCCGTGGTGA
CCGTGCCTTCTTCTTCTTTGGGTACCCAGACCTACATCTGCAACGTGAACCATAAGCCATCCAATACTAAGGTCGACAA
GAAAGTGGAGCCCAAATCCTGCGATAAAACCCATACTTGTCCCCCATGCCCAGCTCCAGAACTGCTCGGAGGGCCCAGT
GTGTTTCTTTTCCCCCCAAAGCCTAAGGACACTCTCATGATATCCCGAACCCCAGAGGTGACATGCGTCGTTGTGGATG
TGTCACATGAAGATCCAGAGGTCAAGTTCAACTGGTATGTCGATGGAGTCGAGGTCCACAATGCTAAGACAAAACCAAG
AGAGGAGCAGTATAACAGTACCTACCGAGTGGTCTCCGTGCTGACTGTTCTTCACCAAGATTGGCTCAATGGTAAAGAG
TACAAATGCAAAGTATCCAACAAGGCCCTTCCTGCACCAATCGAAAAAACTATTTCCAAAGCAAAGGGCCAGCCCAGGG
AGCCACAGGTCTACACACTGCCCCCAAGCAGGGATGAACTCACCAAGAACCAAGTGTCACTTACCTGCCTGGTTAAGGG
ATTCTACCCCTCCGATATTGCAGTAGAATGGGAGTCTAACGGTCAGCCTGAGAATAACTACAAGACCACCCCCCCCGTG
CTCGATAGCGATGGTTCATTCTTCCTGTATAGCAAGCTGACCGTCGATAAGAGTAGGTGGCAACAAGGGAATGTGTTCA
GTTGCTCTGTTATGCACGAAGCCCTCCACAACCATTACACACAAAAGTCCCTTTCCTTGTCTCCCGGTAAG.
(SEQ ID NO: 120)
CAGGTAACACTTAAGGAGAGCGGTCCAGGTATACTCCAGCCTAGTCAGACACTGAGCCTTACATGCAGCTTTAGTGGGT
TCAGCCTGTCCAGTTCTGGCATGTCAGTAGGCTGGATCCGACAACCTAGCGGAAAGGGCCTGGAGTGGCTGGCTCATAT
CTGGTGGTCAGACGACAAGTCCTATAACCCAGCCCTCAAATCCCGTCTGACCATCTCAAAAGACACCAGTAATAATCAG
GTTTTCCTTAAAATCGCCTCTGTGGTCACTGCAGATACAGCAACCTACTACTGTGCACGCGAGGGCTCTTTGGGCTATG
GACTGGACTACTGGGGTCAGGGTACCACTCTGACTGTAAGCAGTGCTTCAACTAAGGGACCTAGCGTGTTTCCACTGGC
ACCCAGTTCTAAATCTACTTCTGGGGGAACTGCTGCTCTGGGATGCCTGGTGAAGGATTACTTTCCAGAGCCCGTCACT
GTGAGTTGGAATAGCGGTGCCTTGACCTCCGGAGTGCACACCTTCCCAGCTGTGCTTCAATCCTCAGGCTTGTATTCTC
TTTCCTCCGTGGTGACCGTGCCATCCTCCTCTCTCGGGACCCAGACTTACATATGTAATGTAAACCACAAGCCCTCTAA
CACAAAGGTAGATAAGAAGGTCGAGCCAAAGTCCTGTGATAAAACACATACATGTCCCCCTTGTCCCGCTCCAGAACTG
CTTGGCGGACCCTCTGTGTTCCTGTTCCCACCTAAGCCAAAAGACACTCTTATGATTTCCAGGACTCCAGAGGTTACAT
GCGTTGTGGTGGACGTAAGCCATGAAGACCCTGAGGTGAAATTCAATTGGTATGTGGACGGTGTGGAGGTCCATAACGC
TAAGACTAAGCCCAGGGAGGAACAGTACAACTCAACATATCGAGTCGTCTCTGTCCTGACAGTGCTCCACCAGGATTGG
CTGAACGGAAAGGAATACAAATGCAAGGTGAGTAATAAAGCCCTGCCAGCTCCCATCGAAAAAACAATCAGCAAGGCTA
AGGGTCAGCCCCGCGAACCACAGGTCTACACTCTGCCACCAAGTCGGGATGAATTGACTAAGAATCAGGTCTCCCTGAC
CTGTCTGGTGAAGGGTTTTTATCCCTCCGACATCGCAGTGGAGTGGGAGAGCAACGGGCAGCCTGAGAATAACTATAAG
ACAACTCCACCCGTCCTCGATTCTGACGGGTCTTTTTTTCTGTACAGCAAACTGACTGTGGACAAGTCTCGGTGGCAGC
AAGGTAACGTCTTCAGCTGCTCCGTTATGCACGAAGCCCTCCACAACCACTACACTCAGAAATCTCTGTCCTTGAGTCC
TGGAAAG.
(SEQ ID NO: 121)
GAAGTTAAACTCCTCGAATCTGGAGGAGGGTTGGTTCAGCCAGGTGGTTCTTTGAAGCTGTCTTGTGCAGCCTCTGGGT
TTGACTTCCGAAGATATTGGATGAGTTGGGTGCGTCAGGCACCAGGGAAAGGTCTTGAATGGATCGGGCAGATTAACCC
ACCCCGACAGTTCAATGATTAACTATACACCTTCTTTGAAAGATAAATTTATTATATCCCGTGACAACGCTAAAAACAC
CTTGTATCTGCAGATGAGTGAGGTGAGATCCGAAGACACTGCTCTTTACTACTGTGCCACCTATGGACACCACGCTACA
GACTCCTGGGGCCAGGGTACCAGTGTGACAGTTTCCAGTGCCAGCACCAAGGGCCCATCCGTTTTCCCCCTGGCTCCCT
CTTCTAAATCTACTTCAGGTGGTACTGCAGCACTTGGATGTTTGGTGAAGGACTACTTCCCTGAGCCAGTCACCGTGAG
TTGGAACTCAGGCGCTCTTACATCCGGTGTCCACACATTTCCAGCCGTGCTGCAGAGCTCAGGACTCTATTCTCTCAGC
TCCGTCGTGACAGTACCCAGCAGCTCACTTGGCACTCAGACCTATATCTGTAATGTCAACCATAAGCCATCTAACACTA
AAGTCGACAAGAAGGTCGAGCCCAAGAGTTGCGACAAAACACATACCTGCCCCCCATGTCCCGCACCAGAACTCCTGGG
TGGTCCAAGCGTGTTTTTGTTTCCACCTAAACCAAAGGACACCCTGATGATTAGCCGTACACCAGAGGTGACTTGCGTG
GTGGTCGACGTGAGTCACGAGGATCCAGAGGTGAAGTTCAACTGGTATGTGGATGGCGTTGAAGTACATAACGCCAAAA
CCAAGCCCAGAGAGGAACAATACAATAGCACCTACCGAGTCGTCAGTGTGCTGACCGTGCTTCACCAAGACTGGCTCAA
TGGGAAAGAGTACAAGTGTAAGGTTAGCAATAAAGCTCTGCCTGCCCCAATCGAGAAGACTATCTCTAAGGCAAAGGGA
CAGCCTAGGGAGCCACAGGTCTATACACTCCCCCCCTCACGGGATGAGTTGACTAAGAATCAGGTGTCACTGACATGCC
TTGTTAAGGGGTTTTATCCCTCTGACATCGCCGTCGAATGGGAGAGTAACGGACAGCCAGAGAACAACTACAAGACAAC
CCCACCAGTCCTGGACTCTGACGGCTCCTTCTTTCTGTATTCAAAGCTGACTGTGGACAAATCTAGATGGCAGCAGGGC
AACGTCTTTAGTTGTAGTGTGATGCATGAGGCTCTGCATAACCATTATACACAGAAAAGTCTCAGTCTTAGCCCAGGAA
AA.
(SEQ ID NO: 122)
CAAGTTCAGTTGCAGCAACCTGGTGCAGAATTGGTGAGACCCGGTACATCTGTCAAGATGAGCTGCAAGGCCAGCGGGT
ACACCTTTACTAGTTACTGGATGCATTGGGTGAAACAGCGACCCGGACAAGGATTGGAGTGGATCGGTGATATCTATCC
AGGGTCCGACAGCACAAACTATCATGAGAAGTTTAAGAGCAAGGCCACCCTGACAGTCGACACCTCAAGCTCCACAGCT
TACATGCAGCTGTCTTCCTTGACTAGCGAAGATTCTGCCGTGTACTATTGCGCTCTTGATAGCTCTGGGTATGGGTTCG
CCTATTGGGGACAGGGTACTCTGGTGACAGTGTCCGCTGCCTCCACCAAAGGTCCCAGTGTATTTCCCTTGGCTCCATC
TAGCAAGTCTACATCCGGCGGTACAGCCGCACTGGGGTGTCTTGTGAAGGACTACTTCCCTGAGCCTGTTACAGTATCT
TGGAATTCAGGCGCTCTGACCTCAGGAGTCCACACCTTTCCCGCTGTCTTGCAGTCTAGCGGCCTTTATAGCCTGTCTT
CCGTGGTGACTGTTCCCTCTAGTAGTCTCGGAACCCAGACCTACATCTGCAATGTGAATCACAAGCCCAGCAACACAAA
AGTCGACAAGAAGGTGGAGCCTAAATCTTGCGACAAGACTCACACCTGTCCTCCATGCCCTGCTCCAGAACTGCTGGGG
GGACCTTCAGTGTTTCTCTTCCCTCCAAAACCAAAGGACACCCTCATGATCTCCCGCACCCCAGAGGTCACCTGTGTGG
TGGTGGACGTTTCACATGAGGATCCTGAGGTGAAGTTCAATTGGTATGTGGACGGTGTCGAAGTTCATAATGCCAAGAC
AAAGCCTCGCGAAGAGCAGTACAACAGCACTTACAGGGTGGTGTCCGTCCTGACTGTCTTGCACCAGGACTGGTTGAAC
GGCAAAGAATACAAATGCAAGGTGAGTAACAAGGCACTGCCTGCACCAATTGAGAAGACTATCTCTAAGGCCAAAGGCC
AGCCTCGTGAGCCTCAAGTGTATACCCTCCCCCCTAGCAGAGACGAGTTGACAAAGAATCAGGTCAGCTTGACATGCCT
GGTGAAGGGATTTTATCCCTCAGACATTGCCGTCGAATGGGAGAGTAACGGCCAGCCAGAAAATAACTACAAGACAACC
CCCCCTGTCCTGGACAGCGATGGATCATTTTTCCTGTACTCAAAGCTGACTGTAGACAAGTCTCGATGGCAGCAAGGTA
ACGTATTCTCCTGCAGCGTGATGCACGAGGCACTGCATAATCACTATACCCAGAAGAGCCTGAGCCTGTCACCAGGGAA
G.
(SEQ ID NO: 123)
GAGGTACAGTTGGTTGAATCTGGCGGTGGACTTGTACAACCAGGCGAATCCTTGAAACTGAGTTGCGACTCTAATGACT
ATGAATTCCCTAGTCACGATATGTCTTGGGTTCGGAAGACCCCTGAGAAACGGCTTGAGCTTGTGGCAGCCATCAACTC
AGACGGTGGAAATACATATTACCCCGACACTATGGAGAGGAGATTTATTATCAGCCGCGATAATACCAAAAGGACACTT
TATCTCCAGATGTCCTCTTTGCGCAGCGAGGATACCGCTCTGCATTATTGTGCCCGCCCACGACCAACTATCGGCACAA
CAGCCACTGGGTCTTCTATGTCTGGGGCACAGGGCCCAAGAAGCGCCTCTACAAAGGGCCCTTCAGTGTTTCCCCTTGC
TCCATCATCTAAAAGTACTAGTGGAGGCACTGCTGCTCTCGGCTGTCTGGTCAAGGACTACTTTCCAGAGCCTGTGACC
GTGTCCTGGAATAGTGGGGCACTTACATCTGGTGTACACACCTTTCCTGCTGTACTGCAGAGCTCTGGCCTGTATTCCC
TGAGCAGCGTTGTCACAGTCCCATCAAGTAGCTTGGGAACTCAGACCTACATATGCAATGTGAATCACAAACCATCCAA
CACCAAGGTGGATAAGAAGGTCGAGCCTAAAAGTTGTGACAAAACCCATACATGCCCACCTTGTCCTGCTCCTGAGCTT
CTGGGCGGCCCAAGTGTTTTCCTTTTCCCTCCCAAACCCAAAGACACACTCATGATTTCCCGTACACCCGAGGTTACTT
GTGTGGTTGTTGATGTCAGCCACGAGGATCCTGAGGTCAAGTTCAACTGGTATGTCGACGGCGTTGAAGTCCACAACGC
TAAGACCAAGCCTCGGGAGGAGCAGTATAATTCCACTTACAGGGTGGTTTCCGTTTTGACCGTGCTGCACCAGGATTGG
CTGAACGGCAAAGAGTATAAATGCAAGGTAAGCAATAAAGCCTTGCCCGCACCCATCGAGAAAACTATTAGCAAAGCCA
AGGGTCAGCCCAGAGAACCTCAAGTTTATACCCTCCCTCCCTCACGCGATGAACTCACTAAGAATCAGGTGTCACTCAC
ATGTCTCGTGAAGGGTTTTTACCCCTCCGATATTGCTGTGGAGTGGGAGTCTAACGGCCAGCCCGAGAACAATTACAAG
ACCACCCCACCTGTGCTTGATAGCGATGGCTCCTTTTTCCTCTACAGTAAGCTGACAGTGGACAAATCTCGGTGGCAGC
AGGGCAACGTTTTCTCCTGTTCAGTAATGCATGAGGCATTGCATAATCATTATACCCAAAAGAGCCTCTCACTTTCCCC
AGGGAAG.
(SEQ ID NO: 124)
TCTATTGTGATGACTCAAAGTCCTAAATTTCTCCTCGTTTCCGCTGGAGACAGGGTGATAATAACTTGCAAAGCTTCAC
AGAGCGTGTCCAATGATGTAGTATGGTACCAGCAAAAGCCAGGCCAGAGCCCAAAACTTCTGATCTACTACGGTTCACA
CCGGAACACCGGAGTACCTACAAGGTTTACTGGTTCCGGCTATGGGACTGACTTCACATTCACTATTTCAACCGTCCAG
GCTGAAGACCTGGCAGTCTATTTTTGTCAGCAGGATTTCTTCTCACCATGGACCTTCGGTGGCGGTACAAAATTGGAGA
TAAAGCGCACTGTTGCCGCCCCTTCCGTCTTTATATTTCCACCCTCCGATGAGCAGCTTAAGTCAGGGACAGCCTCTGT
CGTGTGTCTGCTCAACAATTTTTACCCCCGTGAAGCCAAGGTGCAGTGGAAAGTGGATAACGCCCTGCAGTCTGGCAAT
TCTCAGGAGTCAGTGACCGAACAGGATTCAAAAGACTCTACATATAGCTTGTCATCCACCCTTACTCTGTCCAAAGCAG
ATTATGAGAAGCACAAAGTTTATGCTTGTGAAGTTACTCACCAGGGTCTGAGCAGCCCCGTAACAAAGAGTTTCAACCG
GGGGGAGTGC.
(SEQ ID NO: 125)
GACATAGTTTTGACCCAGTCTCCTGCTAGTCTCGCCGTGTCTCTGGGACAACGAGCAACTATTAGCTGCCGAGCCAGCG
AGAGCGTTGACGAATACGGGATTTCTTTCATGAACTGGTTCCAGCAGAAGCCAGGACAGCCCCCACGTCTGCTTATTTA
CAGCGCCTCTAACCAAGGGAGTGGAGTGCCAGCCAGATTTACAGGTAGTGGATCTGGAACAGATTTCTCCCTCAACATT
CTGCCCATGGAGGAAGACGACACCGCTATGTATTTCTGTCAGCAGAGCAAAGAAGTGCCCTGGACCTTTGGGGGAGGGA
CCAAACTCGAAATTAAGCGAACTGTGGCTGCTCCCTCAGTCTTTATCTTCCCACCATCTGATGAGCAACTGAAGTCTGG
CACTGCATCCGTGGTGTGTCTGCTGAATAATTTTTACCCTCGCGAGGCAAAAGTGCAGTGGAAGGTGGATAATGCTTTG
CAGTCAGGAAATTCTCAGGAGAGTGTTACCGAACAAGATAGCAAGGACTCCACATATAGTTTGTCCTCTACACTTACTC
TCTCCAAGGCAGACTACGAAAAACACAAGGTGTACGCTTGTGAGGTGACCCACCAGGGCCTGAGCTCACCAGTGACCAA
ATCCTTCAACCGCGGCGAGTGC.
(SEQ ID NO: 126)
AGTATTGTGATGACTCAGACCCCCAAGTTTCTTTTGGTGAGTGCAGGTGACAGAGTGACAATTACCTGCAAAGCTTCAC
AGAGCGTCAGCAACGATGTCGTCTGGTACCGACAGAAGCCAGGACAGAGCCCCAAGCTCCTCATCTACTACGCCTCTAA
TCGATACATCGGCGTGCCCGACAGGTTTACCGGTTCTGGCTATGGTACTGATTTTACCTTCACAATTTCTACTGTTCAG
GCCGAAGATCTTGCCGTGTACTTTTGTCAACAGGATTACTATAGTCCCTGGACTTTCGGCGGGGGTACTAAGCTGGAGA
TTAAGCGAACAGTGGCCGCACCCTCCGTGTTTATCTTCCCACCCTCTGACGAGCAACTCAAGAGTGGAACTGCCTCTGT
CGTGTGCTTGCTGAACAACTTTTATCCCAGAGAAGCCAAGGTGCAGTGGAAGGTGGATAATGCCCTGCAGAGCGGGAAC
TCTCAGGAATCCGTCACAGAACAGGATTCAAAGGACTCAACTTATTCCCTTTCTTCAACCCTGACACTCTCCAAGGCCG
ACTACGAGAAGCATAAAGTGTACGCTTGCGAGGTAACCCATCAGGGTCTCAGTAGCCCCGTCACTAAGAGCTTCAACAG
GGGCGAATGC.
(SEQ ID NO: 127)
GATGTGGTTATGACTCAGTCCCCATTGTCCTTGCCAGTCTCTCTGGGTGATCAGGCATCTATTTCCTGCAGGTCCAGCC
AGTCACTGGTGCATTCTAACGGGAACACCTACCTTCACTGGTACTTGCAAAAGCCCGGGCAGAGCCCTAAGCTGCTCAT
TTATAAGGTTTCTAATAGATTCAGCGGTGTGCCTGACAGATTTTCTGGTTCTGGATCTGGCACCGATTTTACCCTGAAG
ATCAGTCGAGTAGAGGCCGAAGACTTGGGAGTGTACTTTTGCTCCCAATCCACCCATGTCCCACCAGATGTGCGATGGC
GTCATCAGGCTGGCAACCAAAGGACTGTGGCTGCCCCTAGTGTTTTTATATTCCCACCTTCAGACGAGCAGCTTAAAAG
TGGCACCGCCTCAGTGGTTTGTTTGCTGAATAATTTCTATCCAAGGGAAGCAAAGGTTCAATGGAAGGTGGACAACGCT
CTGCAAAGCGGAAACAGTCAGGAGAGCGTGACCGAACAGGATAGCAAGGACAGTACTTATAGTTTGAGCAGTACTCTCA
CTTTGTCCAAAGCCGACTACGAGAAGCACAAGGTGTACGCCTGTGAAGTGACCCATCAGGGGTTGTCAAGCCCTGTAAC
TAAAAGTTTTAATAGAGGCGAGTGT.
(SEQ ID NO: 128)
GATATCCAGATGACCCAGACCACTTCCAGTCTTTCTGCTAGTCTCGGCGATCGAGTGACCATCAGCTGCCGAGCATCTC
AGGACATTTCAAATTCACTCAACTGGTATCAACAGAAACCAGATGGCACCGTCAAGCTGCTGATTTATTACACCAGCAG
ACTCCACAGTGGTGTGCCCAGCAGGTTCACAGGTTCTGGGAGTGGGACTGATTACTCCCTGACAATTTCTAACTTGGAA
TTTCTAACTTGGAACAGGAGGATATCGCCACTTATTTTTGCCAGCAGGGCAACACCCTTCCATTGACATTCGGCGCAGG
AACCAAGCTTGAGTTGAAGAGAACCGTCGCCGCACCCTCTGTATTTATATTCCCACCATCTGACGAGCAACTGAAGTCC
GGAACAGCCAGTGTGGTGTGCCTCCTTAATAATTTTTACCCTCGTGAAGCCAAGGTACAGTGGAAGGTTGATAACGCCC
TGCAGAGTGGAAATAGTCAGGAGTCTGTAACCGAGCAGGATTCCAAGGATTCCACATATTCACTGTCCAGTACATTGAC
ATTGTCAAAGGCTGACTACGAAAAGCATAAGGTATACGCCTGCGAAGTGACACATCAGGGGCTGTCTTCTCCCGTGACA
AAGTCATTCAATCGCGGGGAGTGT.
(SEQ ID NO: 129)
GATATTCAGATGAACCAGTCTCCCTCCAGTTTGTCCGCCTCCCTCGGTGATACTATTACTATTACATGCCACGCTAACC
AAAATATCTACGTGTGGCTGTCATGGTATCAACAGAAACCTGGCAACATTCCCAAGCTTCTCATCTACGAGGCATCAAA
CCTGCACACTGGCGTCCCTTCCAGGTTCTCAGGCTCAGGTTCCGGAACCGGTTTTACACTGACCATAAGTAGCCTGCAG
CCTGAAGACATCGCAACCTACTATTGTCAGCAAGGCCAGAGCTACCCCTACACCTTCGGCGGAGGAACAAAGTTGGAGA
TAAAACGGACTGTCGCCGCACCATCTGTCTTCATCTTCCCCCCCAGTGATGAGCAGCTTAAGAGTGGGACTGCTTCTGT
CGTCTGTCTGCTCAATAATTTTTATCCAAGAGAGGCTAAGGTACAGTGGAAAGTGGACAACGCTCTGCAGTCTGGTAAC
TCCCAGGAGAGTGTGACCGAACAGGATTCTAAGGACTCCACATACTCCCTGAGCTCTACACTTACACTCAGTAAAGCAG
ACTATGAGAAGCATAAAGTGTATGCATGTGAGGTGACACACCAGGGTCTGTCCTCTCCTGTGACCAAGTCTTTCAACAG
AGGCGAGTGT.
(SEQ ID NO: 130)
GATATCGTCATGTCACAGAGCCCATCTAGCTTGGCTGTCTCAGTGGGCGAGAAAGTGACCATGTCATGCAAAAGCTCCC
AATCCTTGTTGTACAGCCGCAATCAGAAAAATCGCCTTGCCTGGTACCAGCAGAAACCAGGTCAGTCCCCAAAACTGCT
GATTTATTGGGCCAGCACAAGGGAGAGTGGTGTGCCCGATAGATTCACTGGGTCCGGGTCAGGCACCGATTTCACTCTG
ACCATCTCATCTGTTAAAGCAGAAGATCTGGCCGTCTACTATTGCCAGCAGTACTATAGTTATCCCAGAACTTTCGGGG
GCGGCACCAACCTGGAGATTAAGCGTACCGTGGCAGCACCATCTGTCTTTATTTTCCCCCCATCAGACGAGCAGTTGAA
ATCAGGAACCGCCTCTGTCGTGTGCCTGCTGAACAATTTTTATCCAAGAGAGGCTAAGGTGCAGTGGAAGGTCGATAAT
GCTCTTCAGAGCGGTAATTCTCAAGAATCCGTGACTGAACAGGATTCAAAAGATAGCACATACAGTCTCTCCAGCACCC
TGACCCTCAGCAAGGCCGACTACGAGAAGCACAAAGTGTATGCCTGTGAAGTGACACATCAAGGTCTCTCCAGCCCTGT
AACTAAGAGTTTTAATAGAGGAGAATGC.
(SEQ ID NO: 131)
GATGTCCTGATGACCCAGACTCCATTGAGCCTTCCTGTCTCCTTGGGGGATCAAGCCTCCATTTCATGCAGGAGCTCCC
AGAGCATAGTCCACACAGATGGAAACACTTACCTGGAGTGGTACTTGCAGAAGCCAGGCCAGTCTCCCAAACTGCTCAT
CTACAAAGTGTCTAATAGATTTTCCGGTGTGCCCGACCGTTTTTCAGGTAGCGGCTCTGGTACTGATTTCACTCTGAAG
ATCTCCCGGGTGGAGGCTGAGGATCTGGGCGTCTATTATTGTTTCCAGGGCTCCCACGTGCCATGGACCTTCGGTGGTG
GCACCAAGTTGGAGATTAAGAGGACAGTGGCCGCACCATCCGTGTTCATATTTCCCCCTAGCGATGAACAGTTGAAAAG
CGGTACTGCCTCCGTGGTTTGTCTGCTGAATAATTTCTACCCTAGAGAAGCTAAAGTGCAATGGAAGGTTGACAATGCC
TTGCAGTCAGGCAATTCTCAGGAGTCTGTGACCGAACAGGACAGCAAGGACTCCACCTATTCTCTGTCTTCAACACTGA
CACTTAGTAAGGCTGATTACGAAAAGCATAAGGTATACGCCTGTGAGGTGACACACCAGGGCCTGAGTTCACCAGTGAC
CAAGTCCTTCAATCGCGGGGAGTGT.
(SEQ ID NO: 132)
GATATTGTGCTTACACAGTCCCCAGCTTCCCTGGCCGTTTCCCTTGGGCAGCGAGCCACAATCTCCTGCCGCGCATCTG
AGTCCGTTGATAGCTACGGCAATTCTTTTATGCACTGGTACCAACAAAAACCTGGTCAGCCTCCAAAGTTGCTGATTTA
TCGCGCTTCCAACCTTGAGTCCGGTATTCCTGCTCGCTTTAGCGGCTCTGGGAGTAGAACCGACTTCACACTGACCATT
AACCCAGTTGAGGCTGATGATGTGGCTACCTATTACTGTCAGCAGTCTAACGAAGACCCATTTACATTTGGGTCAGGCA
CTAAGCTGGAGATTAAGCGTACCGTGGCCGCCCCAAGCGTTTTCATCTTCCCCCCAAGCGACGAGCAGCTGAAGTCCGG
AACTGCCTCCGTCGTTTGTCTCTTGAACAATTTTTATCCTAGGGAAGCAAAAGTACAGTGGAAGGTAGATAACGCTCTG
CAAAGCGGTAACTCCCAAGAATCCGTAACCGAACAAGACTCAAAGGACAGTACTTATTCCCTGTCCAGCACCCTGACTC
TTTCTAAAGCTGATTATGAAAAGCATAAAGTTTATGCATGTGAGGTGACCCACCAGGGATTGTCCTCCCCAGTTACTAA
ATCTTTTAACCGGGGGGAATGC.
(SEQ ID NO: 133)
AATATCGTCATGACCCAGACTCCTAAATTTCTTCTGGTCTCCGCTGGAGACCGCGTCACTATAACTCGTAAAGCCAGCC
AGTCCGTGTCTAATGACGTTGTGTGGTACCAGCAGAAGCCCGGGCAGTCCCCTAAGCTGCTCATGTATTACGCAAGTTA
TCGATACACCGGCGTGCCCGATCGATTCACCGGATCCGGTTATGGCACCGACTTCACATTTACTATTTCTAACGTTCAG
GCCGAGGACCTCGCCGTATACTTTTGTCAACAAGACTACTCCAGCCCTTTTACATTTGGAAGCGGGACAAAATTGGAGA
TCAAGAGGACTGTTGCCGCACCTTCCGTGTTTATATTTCCACCTTCCGACGAGCAGCTGAAGTCCGGGACCGCAAGCGT
GGTTTGTCTCCTCAATAATTTCTATCCTCGAGAGGCTAAGGTCCAGTGGAAGGTGGACAATGCTCTGCAGAGTGGCAAC
TCTCAGGAGTCAGTTACTGAACAAGACAGCAAGGACAGCACATATTCACTGTCATCCACTCTTACTCTCTCCAAGGCTG
ACTACGAAAAGCATAAGGTTTATGCCTGCGAGGTTACTCATCAGGGTCTGTCCAGTCCAGTGACCAAATCTTTTAATAG
AGGAGAGTGC.
(SEQ ID NO: 134)
GATATTGTCTTGACCCAATCACCTGCTAGCCTGGCTGTGAGCTTGGGGCAGCGAGCAACCATCTCCTGTAGGGCCTCTC
AGTCCGTCTCCACAAGTACTTACAACTACATGCATTGGTACCAGCAGAAACCTGGCCAACCACCAAAATTGCTCATTAA
GTATGCTAGCAACCTTGAGTCAGGCGTCCCCGCAAGGTTTAGCGGATCTGGCTCAGGAACCGATTTTACTCTGAACATT
CACCCCGTGGAGGAGGAGGACACTGCTACCTACTATTGCCAGCATAGTTGGGAGATACCCTTCACCTTTGGCAGTGGCA
CAAAGCTCGAGATTAAGCGCACTGTTGCCGCACCCAGCGTGTTTATCTTTCCACCTAGCGACGAACAGCTTAAGTCTGG
TACTGCTAGTGTGGTGTGTTTGTTGAATAACTTCTACCCCCGTGAAGCCAAGGTTCAGTGGAAAGTGGATAACGCTCTC
CAGAGCGGGAACAGTCAGGAGTCTGTTACCGAACAGGACTCAAAGGACAGCACTTACAGCCTGAGCAGTACTCTGACCC
TGAGTAAGGCCGATTATGAAAAGCACAAGGTGTACGCCTGTGAGGTGACCCACCAGGGTCTGTCATCCCCTGTCACCAA
GTCTTTTAATCGGGGTGAATGC.

Wherein, in the present application, the nucleotide sequences shown in the above SEQ ID NO: 113 and 124 respectively encode the heavy chain and light chain of 3-6A4 antibody, the nucleotide sequences shown in the above SEQ ID NO: 114 and 125 respectively encode the heavy chain and light chain of 5-20E11-5F2 antibody, the nucleotide sequences shown in the above SEQ ID NO: 115 and 126 respectively encode the heavy chain and light chain of 3-5B2-2G4 antibody, the nucleotide sequences shown in the above SEQ ID NO: 116 and 127 respectively encode the heavy chain and light chain of 3-5B2-7A4 antibody, the nucleotide sequences shown in the above SEQ ID NO: 117 and 128 respectively encode the heavy chain and light chain of 3-2B5-3D2 antibody, the nucleotide sequences shown in the above SEQ ID NO: 118 and SEQ ID NO:129 respectively encode the heavy chain and light chain of 3-7C9-1A6 antibody, the nucleotide sequences shown in the above SEQ ID NO: 119 and SEQ ID NO:130 respectively encode the heavy chain and light chain of 3-9E1-3A2 antibody, the nucleotide sequences shown in the above SEQ ID NO: 120 and SEQ ID NO:131 respectively encode the heavy chain and light chain of 3-15D5-3A6 antibody, the nucleotide sequences shown in the above SEQ ID NO: 121 and SEQ ID NO:132 respectively encode the heavy chain and light chain of 3-15D5-6A1 antibody, the nucleotide sequences shown in the above SEQ ID NO: 122 and SEQ ID NO:133 respectively encode the heavy chain and light chain of 5-20E11-1A2 antibody, the nucleotide sequences shown in the above SEQ ID NO: 123 and SEQ ID NO:134 respectively encode the heavy chain and light chain of 5-21G2-1D3 antibody.

According to the embodiments of the present invention, the nucleic acid molecule has a nucleotide sequence shown in any one of SEQ ID NO: 159-162 or has a nucleotide sequence shown in any one of SEQ ID NO: 163-166.

(SEQ ID NO: 159)
CAAGTGCAGCTGGTTCAGAGCGGAGCTGAGGTGAAGAAGCCTGGAGCTAGCGTGAAGGTGTCTTGTAAAGCCAGCGA
CTACACCTTTACCAATTACTGGCTGGGCTGGGTCAGACAGGCCCCTGGCCAGGGCCTGGAATGGATGGGCCACATCT
ACCCCGGCGGAGGCTACACAAACTACAACGAGAAGTTCAAGGGCAGAGTGACAATGACCAGAGATACAAGCACCAGC
ACAGTGTATATGGAACTGTCTAGCCTGCGGAGCGAGGACACCGCCGTGTACTACTGCGCCAGAGTGCGGTCCGGCAA
CGACGCCCTGGATTTCTGGGGCCAGGGCACCCTGGTGACCGTGTCCAGCGCCAGCACCAAGGGCCCTTCCGTGTTTC
CACTGGCCCCCTCCTCTAAATCCACATCTGGCGGCACCGCCGCCCTGGGCTGTCTGGTGAAGGACTACTTCCCAGAG
CCTGTGACAGTGTCCTGGAACTCTGGCGCCCTGACATCCGGCGTGCACACATTTCCAGCCGTGCTGCAGAGCTCCGG
CCTGTACAGCCTGTCTAGCGTGGTGACAGTGCCCTCCTCTAGCCTGGGCACACAGACCTATATCTGCAACGTGAATC
ACAAGCCAAGCAATACCAAGGTGGACAAGAAGGTGGAGCCCAAGTCCTGTGATAAGACACACACCTGCCCCCCTTGT
CCTGCTCCCGAGCTGCTGGGCGGCCCTAGCGTGTTCCTGTTTCCACCCAAGCCTAAGGACACCCTGATGATCTCCCG
GACACCCGAGGTGACCTGCGTGGTGGTGGACGTGTCTCACGAGGATCCTGAGGTGAAGTTCAACTGGTATGTGGATG
GCGTGGAGGTGCACAATGCCAAGACCAAGCCCAGAGAGGAGCAGTACAACTCTACATATAGGGTGGTGAGCGTGCTG
ACCGTGCTGCACCAGGACTGGCTGAACGGCAAGGAGTATAAGTGCAAGGTGTCCAATAAGGCCCTGCCCGCCCCCAT
CGAGAAGACAATCAGCAAGGCCAAGGGCCAGCCTCGGGAGCCACAGGTGTACACCCTGCCTCCATCCAGAGACGAGC
TGACAAAGAACCAGGTGTCTCTGACATGTCTGGTGAAGGGCTTCTATCCTAGCGATATCGCCGTGGAGTGGGAGTCC
AATGGCCAGCCAGAGAACAATTACAAGACCACACCCCCTGTGCTGGACTCCGATGGCTCCTTCTTTCTGTATTCCAA
GCTGACCGTGGATAAGTCTCGGTGGCAGCAGGGCAACGTGTTCAGCTGTTCCGTGATGCACGAAGCCCTGCATAATC
ACTATACTCAGAAATCCCTGTCCCTGTCACCTGGAAAGTGATAA.
(SEQ ID NO: 160)
CAAGTGCAGCTGGTCCAGAGCGGCGCCGAGGTGAAAAAGCCTGGAGCTAGCGTGAAGGTGTCCTGCAAGGCCAGCGA
CTACACCTTCACCAACTATTGGCTGGGCTGGGTGCGGCAGGCCCCTGGACAGGGCCTGGAATGGATCGGCCACATCT
ACCCCGGAGGCGGCTACACCAATTACAACGAGAAGTTCAAGGGCAGAGTGACCATGACCGCCGATACAAGCACATCT
ACAGTGTACATGGAACTGAGCAGCCTGAGAAGCGAGGATACAGCTGTTTACTTCTGTGCCAGAGTGCGGTCCGGCAA
CGACGCCCTGGACTTTTGGGGCCAGGGCACCCTGGTGACCGTGTCTAGCGCCAGCACCAAGGGCCCTTCCGTGTTTC
CACTGGCCCCCTCCTCTAAATCCACATCTGGCGGCACCGCCGCCCTGGGCTGTCTGGTGAAGGACTACTTCCCAGAG
CCTGTGACAGTGTCCTGGAACTCTGGCGCCCTGACATCCGGCGTGCACACATTTCCAGCCGTGCTGCAGAGCTCCGG
CCTGTACAGCCTGTCTAGCGTGGTGACAGTGCCCTCCTCTAGCCTGGGCACACAGACCTATATCTGCAACGTGAATC
ACAAGCCAAGCAATACCAAGGTGGACAAGAAGGTGGAGCCCAAGTCCTGTGATAAGACACACACCTGCCCCCCTTGT
CCTGCTCCCGAGCTGCTGGGCGGCCCTAGCGTGTTCCTGTTTCCACCCAAGCCTAAGGACACCCTGATGATCTCCCG
GACACCCGAGGTGACCTGCGTGGTGGTGGACGTGTCTCACGAGGATCCTGAGGTGAAGTTCAACTGGTATGTGGATG
GCGTGGAGGTGCACAATGCCAAGACCAAGCCCAGAGAGGAGCAGTACAACTCTACATATAGGGTGGTGAGCGTGCTG
ACCGTGCTGCACCAGGACTGGCTGAACGGCAAGGAGTATAAGTGCAAGGTGTCCAATAAGGCCCTGCCCGCCCCCAT
CGAGAAGACAATCAGCAAGGCCAAGGGCCAGCCTCGGGAGCCACAGGTGTACACCCTGCCTCCATCCAGAGACGAGC
TGACAAAGAACCAGGTGTCTCTGACATGTCTGGTGAAGGGCTTCTATCCTAGCGATATCGCCGTGGAGTGGGAGTCC
AATGGCCAGCCAGAGAACAATTACAAGACCACACCCCCTGTGCTGGACTCCGATGGCTCCTTCTTTCTGTATTCCAA
GCTGACCGTGGATAAGTCTCGGTGGCAGCAGGGCAACGTGTTCAGCTGTTCCGTGATGCACGAAGCCCTGCATAATC
ACTATACTCAGAAATCCCTGTCCCTGTCACCTGGAAAGTGATAA.
(SEQ ID NO: 161)
CAAGTGCAGCTGGTCCAGAGCGGCGCCGAGGTGAAAAAGCCTGGCGCTTCTGTGAAGGTGTCCTGCAAGGCCAGCGA
CTACACATTTACCAATTATTGGCTGGGCTGGGTGCGGCAGGCCCCTGGACAGGGCCTGGAATGGATCGGCCACATCT
ACCCCGGCGGAGGCTACACAAACTACAACGAGAAGTTCAAGGGCAGAGCCACACTCACCGCTGATACCAGCACAAGC
ACCGTGTACATGGAACTGAGCAGCCTGAGAAGCGAGGACACCGCCGTGTACTTCTGTGCCAGAGTGCGGTCCGGCAA
CGACGCCCTGGATTTCTGGGGACAGGGCACCCTGGTGACCGTGTCTAGCGCCAGCACCAAGGGCCCTTCCGTGTTTC
CACTGGCCCCCTCCTCTAAATCCACATCTGGCGGCACCGCCGCCCTGGGCTGTCTGGTGAAGGACTACTTCCCAGAG
CCTGTGACAGTGTCCTGGAACTCTGGCGCCCTGACATCCGGCGTGCACACATTTCCAGCCGTGCTGCAGAGCTCCGG
CCTGTACAGCCTGTCTAGCGTGGTGACAGTGCCCTCCTCTAGCCTGGGCACACAGACCTATATCTGCAACGTGAATC
ACAAGCCAAGCAATACCAAGGTGGACAAGAAGGTGGAGCCCAAGTCCTGTGATAAGACACACACCTGCCCCCCTTGT
CCTGCTCCCGAGCTGCTGGGCGGCCCTAGCGTGTTCCTGTTTCCACCCAAGCCTAAGGACACCCTGATGATCTCCCG
GACACCCGAGGTGACCTGCGTGGTGGTGGACGTGTCTCACGAGGATCCTGAGGTGAAGTTCAACTGGTATGTGGATG
GCGTGGAGGTGCACAATGCCAAGACCAAGCCCAGAGAGGAGCAGTACAACTCTACATATAGGGTGGTGAGCGTGCTG
ACCGTGCTGCACCAGGACTGGCTGAACGGCAAGGAGTATAAGTGCAAGGTGTCCAATAAGGCCCTGCCCGCCCCCAT
CGAGAAGACAATCAGCAAGGCCAAGGGCCAGCCTCGGGAGCCACAGGTGTACACCCTGCCTCCATCCAGAGACGAGC
TGACAAAGAACCAGGTGTCTCTGACATGTCTGGTGAAGGGCTTCTATCCTAGCGATATCGCCGTGGAGTGGGAGTCC
AATGGCCAGCCAGAGAACAATTACAAGACCACACCCCCTGTGCTGGACTCCGATGGCTCCTTCTTTCTGTATTCCAA
CACCCCCTGTGCTGGACTCCGATGGCTCCTTCTTTCTGTATTCCAAGCTGACCGTGGATAAGTCTCGGTGGCAGCAG
GGCAACGTGTTCAGCTGTTCCGTGATGCACGAAGCCCTGCATAATCACTATACTCAGAAATCCCTGTCCCTGTCACC
TGGAAAGTGATAA.
(SEQ ID NO: 162)
CAAGTGCAGCTGGTTCAGAGCGGCGCCGAGGTGAAGAAGCCTGGCGCTAGCGTGAAGGTGTCTTGTAAAGCCAGCGA
CTACACATTTACCAATTACTGGCTGGGATGGGTCCGGCAGGCCCCTGGCCACGGCCTGGAATGGATCGGCCACATCT
ACCCCGGCGGAGGATATACAAACTACAACGAGAAGTTCAAGGGCAGAGCCACCCTGACCGCTGATACATCTACAAGC
ACCGTGTACATGGAACTGAGCAGCCTGAGAAGCGAGGATACCGCCGTGTACTTCTGCGCCAGAGTGCGGTCCGGCAA
CGACGCCCTGGACTTCTGGGGCCAGGGCACCCTGGTGACCGTGTCCAGCGCCAGCACCAAGGGCCCTTCCGTGTTTC
CACTGGCCCCCTCCTCTAAATCCACATCTGGCGGCACCGCCGCCCTGGGCTGTCTGGTGAAGGACTACTTCCCAGAG
CCTGTGACAGTGTCCTGGAACTCTGGCGCCCTGACATCCGGCGTGCACACATTTCCAGCCGTGCTGCAGAGCTCCGG
CCTGTACAGCCTGTCTAGCGTGGTGACAGTGCCCTCCTCTAGCCTGGGCACACAGACCTATATCTGCAACGTGAATC
ACAAGCCAAGCAATACCAAGGTGGACAAGAAGGTGGAGCCCAAGTCCTGTGATAAGACACACACCTGCCCCCCTTGT
CCTGCTCCCGAGCTGCTGGGCGGCCCTAGCGTGTTCCTGTTTCCACCCAAGCCTAAGGACACCCTGATGATCTCCCG
GACACCCGAGGTGACCTGCGTGGTGGTGGACGTGTCTCACGAGGATCCTGAGGTGAAGTTCAACTGGTATGTGGATG
GCGTGGAGGTGCACAATGCCAAGACCAAGCCCAGAGAGGAGCAGTACAACTCTACATATAGGGTGGTGAGCGTGCTG
ACCGTGCTGCACCAGGACTGGCTGAACGGCAAGGAGTATAAGTGCAAGGTGTCCAATAAGGCCCTGCCCGCCCCCAT
CGAGAAGACAATCAGCAAGGCCAAGGGCCAGCCTCGGGAGCCACAGGTGTACACCCTGCCTCCATCCAGAGACGAGC
TGACAAAGAACCAGGTGTCTCTGACATGTCTGGTGAAGGGCTTCTATCCTAGCGATATCGCCGTGGAGTGGGAGTCC
AATGGCCAGCCAGAGAACAATTACAAGACCACACCCCCTGTGCTGGACTCCGATGGCTCCTTCTTTCTGTATTCCAA
GCTGACCGTGGATAAGTCTCGGTGGCAGCAGGGCAACGTGTTCAGCTGTTCCGTGATGCACGAAGCCCTGCATAATC
ACTATACTCAGAAATCCCTGTCCCTGTCACCTGGAAAGTGATAA.
(SEQ ID NO: 163)
GAGATCGTGCTGACACAGAGCCCTGCTACACTCTCCCTGTCCCCAGGCGAGCGGGCCACACTGTCTTGTAGAGCCAG
CGAGAGCGTCGACGAGTACGGCATTAGCTTCATGAACTGGTATCAGCAGAAACCTGGCCAGGCCCCTAGACTGCTGA
TCTACAGCGCCTCCAACCAGGGCAGCGGCATCCCCGCTAGATTCAGCGGCAGCGGATCTGGCACCGACTTCACCCTG
ACCATCAGCAGCCTGGAACCCGAGGATTTTGCCGTGTACTACTGCCAGCAATCTAAGGAAGTGCCTTGGACCTTCGG
CGGAGGCACCAAGCTGGAAATCAAGAGGACAGTGGCCGCCCCAAGCGTGTTCATCTTTCCCCCTTCCGACGAGCAGC
TGAAGTCTGGCACCGCCAGCGTGGTGTGCCTGCTGAACAACTTCTACCCTCGGGAGGCCAAGGTCCAGTGGAAGGTG
GATAACGCCCTGCAGTCTGGCAATAGCCAGGAGTCCGTGACCGAGCAGGACTCTAAGGATAGCACATATTCCCTGTC
TAGCACCCTGACACTGAGCAAGGCCGATTACGAGAAGCACAAGGTGTATGCCTGTGAAGTCACCCATCAGGGGCTGT
CATCACCCGTCACTAAGTCATTCAATCGCGGAGAATGCTGATAA.
(SEQ ID NO: 164)
GAGATCGTGCTGACCCAGAGCCCCGCCACCCTGTCCCTGAGCCCTGGCGAGCGGGCTACACTGTCTTGTAGAGCCAG
CGAGAGCGTCGACGAGTACGGCATCTCTTTTATGAACTGGTATCAGCAGAAACCTGGACAGGCCCCTAGACTGCTGA
TCTACAGCGCCTCCAACCAGGGCAGCGGCATTCCTGCTAGATTCAGCGGCAGCGGCTCCGGCACAGATTTCACCCTC
ACCATCAGCAGCCTGGAACCCGAGGACTTCGCCGTGTACTTCTGCCAGCAATCTAAGGAAGTGCCATGGACATTCGG
CGGAGGCACCAAGCTGGAAATCAAGAGGACAGTGGCCGCCCCAAGCGTGTTCATCTTTCCCCCTTCCGACGAGCAGC
TGAAGTCTGGCACCGCCAGCGTGGTGTGCCTGCTGAACAACTTCTACCCTCGGGAGGCCAAGGTCCAGTGGAAGGTG
GATAACGCCCTGCAGTCTGGCAATAGCCAGGAGTCCGTGACCGAGCAGGACTCTAAGGATAGCACATATTCCCTGTC
TAGCACCCTGACACTGAGCAAGGCCGATTACGAGAAGCACAAGGTGTATGCCTGTGAAGTCACCCATCAGGGGCTGT
CATCACCCGTCACTAAGTCATTCAATCGCGGAGAATGCTGATAA.
(SEQ ID NO: 165)
GAGATCGTGCTGACCCAGTCTCCAGCCACCCTCTCCCTGAGCCCCGGCGAGCGGGCCACACTGTCTTGTAGAGCTAG
CGAGAGCGTCGACGAGTACGGCATCTCCTTCATGAACTGGTTCCAGCAGAAACCTGGCCAGGCCCCTAGACTGCTGA
TCTACAGCGCCAGCAACCAGGGCTCCGGAATTCCTGCTAGATTCAGCGGCTCTGGCAGCGGCACAGATTTTACCCTG
ACAATCAGCAGCCTGGAACCCGAGGACTTTGCCGTGTACTTCTGCCAGCAAAGCAAGGAAGTGCCTTGGACCTTCGG
CGGAGGCACCAAGCTGGAAATCAAGAGGACAGTGGCCGCCCCAAGCGTGTTCATCTTTCCCCCTTCCGACGAGCAGC
TGAAGTCTGGCACCGCCAGCGTGGTGTGCCTGCTGAACAACTTCTACCCTCGGGAGGCCAAGGTCCAGTGGAAGGTG
GATAACGCCCTGCAGTCTGGCAATAGCCAGGAGTCCGTGACCGAGCAGGACTCTAAGGATAGCACATATTCCCTGTC
TAGCACCCTGACACTGAGCAAGGCCGATTACGAGAAGCACAAGGTGTATGCCTGTGAAGTCACCCATCAGGGGCTGT
CATCACCCGTCACTAAGTCATTCAATCGCGGAGAATGCTGATAA.
(SEQ ID NO: 166)
GAGATCGTGCTGACACAGAGCCCTGCTACACTCTCCCTGAGCCCCGGCGAGCGGGCCACCCTGTCTTGTAGAGCCAG
CGAGAGCGTGGACGAGTACGGCATTAGCTTCATGAACTGGTTTCAGCAGAAACCTGGCCAGGCCCCTAGACTGCTGA
TCTACAGCGCCTCCAACCAGGGCTCTGGCATCCCCGCTAGATTCAGCGGATCTGGCTCCGGCACCGACTTCACCCTG
ACCATCAGCAGCCTGGAACCAGAGGATTTTGCCATGTACTTCTGCCAGCAAAGCAAGGAAGTGCCTTGGACCTTCGG
CGGAGGCACAAAGCTGGAAATCAAGAGGACAGTGGCCGCCCCAAGCGTGTTCATCTTTCCCCCTTCCGACGAGCAGC
TGAAGTCTGGCACCGCCAGCGTGGTGTGCCTGCTGAACAACTTCTACCCTCGGGAGGCCAAGGTCCAGTGGAAGGTG
GATAACGCCCTGCAGTCTGGCAATAGCCAGGAGTCCGTGACCGAGCAGGACTCTAAGGATAGCACATATTCCCTGTC
TAGCACCCTGACACTGAGCAAGGCCGATTACGAGAAGCACAAGGTGTATGCCTGTGAAGTCACCCATCAGGGGCTGT
CATCACCCGTCACTAAGTCATTCAATCGCGGAGAATGCTGATAA.

In the third aspect of the present invention, the present invention provides an expression vector. According to the embodiments of the present invention, the expression vector carries the aforementioned nucleic acid molecule. After the expression vector according to the embodiments of the present invention is introduced into a suitable recipient cell, the expression of the aforementioned antibody or antigen-binding fragment thereof that specifically recognizes IL-11 can be effectively realized under the mediation of the regulatory system, thereby in vitro mass acquisition of the antibody or antigen-binding fragment are achieved.

According to the embodiments of the present invention, the above-mentioned expression vector may further include at least one of the following additional technical features:

According to the embodiments of the present invention, the expression vector is a eukaryotic expression vector. Thereby the aforementioned antibody or antigen-binding fragment thereof that specifically recognizes IL-11 can be expressed in eukaryotic cells, such as CHO cells.

In the fourth aspect of the present invention, the present invention provides a recombinant cell. According to the embodiments of the present invention, the recombinant cell carries the aforementioned nucleic acid molecule, or expresses the aforementioned antibody or antigen-binding fragment thereof. The recombinant cell according to the embodiments of the present invention can be used for the in vitro expression and mass acquisition of the aforementioned antibody or antigen-binding fragment thereof that specifically recognizes IL-11.

According to the embodiments of the present invention, the above-mentioned recombinant cell may further include at least one of the following additional technical features:

According to the embodiments of the present invention, the recombinant cell is obtained by introducing the aforementioned expression vector into a host cell.

According to the embodiments of the present invention, the expression vector is introduced into the host cell by electrotransduction.

According to the embodiments of the present invention, the recombinant cell is a eukaryotic cell.

According to the embodiments of the present invention, the recombinant cell is a mammalian cell.

In the fifth aspect of the present invention, the present invention provides a pharmaceutical composition. According to the embodiments of the present invention, the pharmaceutical composition comprises the aforementioned antibody, the aforementioned nucleic acid molecule, the aforementioned expression vector or the aforementioned recombinant cell. The antibody contained or expressed in the pharmaceutical composition according to the embodiments of the present invention can specifically target and bind to IL-11, and block the binding of IL-11 to the IL-11 receptor.

In the sixth aspect of the present invention, the present invention provides use of the aforementioned antibody, the aforementioned nucleic acid molecule, the aforementioned expression vector or the aforementioned recombinant cell, and the aforementioned pharmaceutical composition in the manufacture of a medicament, the medicament is used for treating pulmonary fibrosis, non-alcoholic steatohepatitis (NASH), chronic heart failure (CHF) or diseases caused by the transformation of fibroblasts into fibrosis.

In the seventh aspect of the present invention, the present invention provides a kit for detecting IL-11. According to the embodiments of the present invention, the kit includes any one of the aforementioned antibodies. The aforementioned IL-11 antibody can specifically target and bind to IL-11. The kit according to the embodiments of the present invention can realize the specific detection of IL-11. For example, when the antibody is bound with a fluorophore, a fluorescence detection device can be used to achieve localization or real-time detection of IL-11.

In the eighth aspect of the present invention, the present invention provides use of the aforementioned antibody, the aforementioned nucleic acid molecule, the aforementioned expression vector or the aforementioned recombinant cell in the preparation of a kit, the kit is used for detecting IL-11 or diagnosing IL-11-related diseases.

Additional aspects and advantages of the present invention will be set forth in part from the following description, and in part will be apparent from the following description, or may be learned by practice of the invention.

### DESCRIPTION OF THE DRAWINGS

The above and/or additional aspects and advantages of the present invention will become apparent and readily understood from the following description of embodiments taken in conjunction with the accompanying drawings, wherein:

FIG. 1 is the result of competitive blocking the binding of IL-11 and IL-11R by a preferred antibody according to the embodiments of the present invention;

FIG. 2 is the test result of competitive blocking activity of the preferred antibody according to the embodiments of the present invention;

FIG. 3 and FIG. 4 are the western test results of the preferred antibodies inhibiting the transformation of lung fibroblast HFL-1 to fibrosis according to the embodiments of the present invention;

FIG. 5 is the statistical analysis result of the preferred antibodies inhibiting the transformation of lung fibroblasts HFL-1 to fibrosis according to the embodiments of the present invention;

FIG. 6 and FIG. 7 are results of inhibition of pulmonary fibrosis in C57BL/6 mice by the preferred antibody according to the embodiments of the present invention;

FIGS. 8, 9, 10 and 11 are results of inhibition of pulmonary fibrosis in Sprague Dawley rats by the preferred antibody according to the embodiments of the present invention; and

FIG. 12 is the competitive blocking activity of the humanized antibody according to the embodiments of the present invention.

### EXAMPLES

The embodiments of the present invention are described in detail below, examples of the embodiments are illustrated in the accompanying drawings. The embodiments described below with reference to the accompanying drawings are exemplary, and are intended to explain the present invention and should not be construed as limiting the present invention.

In addition, the terms “first” and “second” are only used for descriptive purposes, and should not be construed as indicating or implying relative importance or implying the number of indicated technical features. Thus, a feature delimited with “first”, “second” may expressly or implicitly include at least one of that feature. In the description of the present invention, “plurality” means at least two, such as two, three, etc., unless otherwise expressly and specifically defined.

Antibody

As used herein, the term “antibody” is an immunoglobulin molecule capable of binding to a specific antigen. It consists of two light chains with lighter molecular weight and two heavy chains with heavier molecular weight. The heavy chain (H chain) and the light chain (L chain) are connected by a disulfide bond to form a tetrapeptide chain molecule. Among them, the amino acid sequence of the amino-terminal (N-terminus) of the peptide chain varies greatly, which is called variable region (V region), and the carboxyl-terminal (C-terminus) is relatively stable and varies little, which is called constant region (C region). The V regions of the L chain and H chain are called VL and VH, respectively.

The amino acid composition and arrangement sequence of certain regions in the variable region have a higher degree of variation, which is called hypervariable region (HVR). The hypervariable region is where the antigen and antibody bind, so it is also called complementarity-determining region (CDR). There are three CDR regions on both the heavy chain variable region and the light chain variable region. The amino acid composition and arrangement sequence outside the hypervariable region of the antibody variable region vary relatively little, which is called framework region (FR). There are four framework regions in VH and VL respectively, which are represented by FR1, FR2, FR3 and FR4, respectively.

The present invention uses an IL-11 antigen to obtain a highly specific and high-affinity anti-IL-11 Fab (antigen-binding fragment) antibody fragment through immunization. The antibody fragment can specifically bind to the IL-11 antigen, so that diseases such as pulmonary fibrosis can be targeted for treatment.

It should be noted that “immunogenicity” refers to the property that can cause an immune response, that is, an antigen can stimulate specific immune cells, to make the immune cells activate, proliferate, and differentiate, and finally produce the characteristics of immune effector antibodies and sensitized lymphocytes. The inventors of the present application further carried out humanization transformation (humanization modification) on the screened new neutralizing antibody sequences of murine origin to obtain a humanized antibody, which can not only retain the affinity and specificity of the parental mouse monoclonal antibody, but also greatly reduce its immunogenicity and improve its safety.

In some embodiments, the present invention provides an antibody or antigen-binding fragment that can specifically recognize IL-11, the antibody comprises CDRs selected from at least one of the following or an amino acid sequence having at least 95% identity thereto: a heavy chain variable region comprising CDRs having the amino acid sequence of SEQ ID NO: 1-33, a light chain variable region comprising CDRs having the amino acid sequence of SEQ ID NO: 34-66. In other embodiments, the antibody or antigen-binding fragment provided by the present invention has conservative amino acid substitution compared to the above heavy chain and light chain. An “antigen-binding fragment” refers to an antibody fragment that retains the ability to specifically bind an antigen. “Conservative amino acid substitution” refers to the replacement of an amino acid by another amino acid with a residue that is biologically, chemically, or structurally similar. Biologically similar means that the substitution does not destroy the biological activity of the IL-11 antibody or binding to the IL-11 antigen. Structurally similar means that amino acids have side chains of similar length, such as alanine, glycine, or serine, or have side chains of similar size. Chemically similar means that amino acids have the same charge or are both hydrophilic or hydrophobic. For example, the hydrophobic residues isoleucine, valine, leucine or methionine are substituted for each other. Alternatively, polar amino acids can be substituted for each other, e.g., arginine substituted for lysine, glutamate substituted for aspartic acid, glutamine substituted for asparagine, serine substituted for threonine, and the like.

In some embodiments, the present invention provides an antibody or antigen-binding fragment, the heavy chain variable region has the amino acid sequence shown in any one of SEQ ID NO: 67-77 and the light chain variable region has the amino acid sequence shown in any one of SEQ ID NO: 78-88. The inventors can obtain the CDR regions of the above-mentioned heavy chain variable region (as shown in SEQ ID NO: 1-33) and the CDR regions of the light chain variable region (as shown in SEQ ID NO: 34-66) through the antibody sequence alignment database (NCBI, IMGT). In other embodiments, the heavy chain variable region of the antibody or antigen-binding fragment have conservative amino acid substitution compared to the amino acid sequence shown in SEQ ID NO: 67-77. In other embodiments, the light chain variable region of the antibody or antigen-binding fragment have conservative amino acid substitution compared to the amino acid sequence shown in any one of SEQ ID NO: 78-88. Of course, these conservative amino acid substitutions do not alter the biological function of the antibody or antigen-binding fragment. In some embodiments, these conservative amino acid substitutions can occur at the amino acids in heavy and light chain variable regions other than the CDR regions.

In some preferred embodiments, the present invention provides an anti-IL-11 antibody, the heavy chain has the amino acid sequence shown in any one of SEQ ID NO: 91-101 and the light chain has the amino acid sequence shown in any one of SEQ ID NO: 102-112.

In some preferred embodiments, the present invention provides an anti-IL-11 single chain antibody.

Nucleic Acid Molecule, Expression Vector, Recombinant Cell

In the process of preparing or obtaining these antibodies, the nucleic acid molecules expressing these antibodies can be used to connect with different vectors, and then express in different cells to obtain corresponding antibodies.

To this end, the present invention also provides an isolated nucleic acid molecule, which encodes the antibody or antigen-binding fragment described above.

In some embodiments, the isolated nucleic acid molecule has the nucleotide sequence shown in any one of SEQ ID NO: 113-123 or has the nucleotide sequence shown in any one of SEQ ID NO: 124-134.

In some embodiments, the isolated nucleic acid molecule has at least more than 90% homology with the nucleotide sequences shown in SEQ ID NO: 113-123 above, preferably, more than 95% homology, more preferably, more than 98% or 99% homology. In at least some embodiments, the isolated polynucleotide has at least more than 90% homology with the nucleotide sequences shown in SEQ ID NO: 124-134, preferably, more than 95% homology, more preferably, more than 98% or 99% homology. These sequences have homology to the nucleotide sequences shown in SEQ ID NO: 113-123 or SEQ ID NO: 124-134, and can express amino acids similar to SEQ ID NO: 91-101 and SEQ ID NO: 102-112, thereby can specifically bind to the IL-11 antigen to achieve the targeting function of the antibody.

In some preferred embodiments, the isolated nucleic acid molecule has a heavy chain nucleotide sequence shown in SEQ ID NO: 113-123 and a light chain nucleotide sequence shown in SEQ ID NO: 124-134. These nucleotide sequences are species optimized for easier expression in mammalian cells.

The present invention also provides an expression vector, which comprises the above-described isolated nucleic acid molecule. When the above-described isolated polynucleotide is ligated to a vector, the polynucleotide can be directly or indirectly connected to control elements on the vector, as long as these control elements can control the translation and expression and the like of the polynucleotide. Of course, these control elements can come directly from the vector itself, or can be exogenous, that is, not from the vector itself. Of course, it is sufficient that the polynucleotide is operably linked to the control elements. Herein, “operably linked” refers to linking an exogenous gene to a vector, so that the control elements in the vector, such as transcriptional control sequences and translational control sequences, etc., can perform the intended functions of regulating the transcription and translation of the exogenous gene. Of course, the polynucleotides used to encode the heavy chain and the light chain of the antibody can be independently inserted into different vectors, and usually inserted into the same vector. Commonly used vectors can be, for example, plasmid, phage, and the like. For example, Plasmid-X plasmid.

The present invention also provides a recombinant cell, which comprises the expression vector. The expression vector can be introduced into mammalian cells to construct and obtain recombinant cells, and then these recombinant cells can be used to express the antibody or antigen-binding fragment provided by the present invention. By culturing the recombinant cells, corresponding antibodies can be obtained. These usable mammalian cells can be, for example, CHO cells and the like.

Pharmaceutical Composition, Kit and Pharmaceutical Use and Use in the Preparation of Kit.

The present invention also provides a pharmaceutical composition, which comprises the above-mentioned antibody or antigen-binding fragment and a pharmaceutically acceptable carrier.

The anti-IL-11 antibody provided herein can be incorporated into pharmaceutical compositions suitable for administration to a subject. Typically, these pharmaceutical compositions include an anti-IL-11 antibody provided herein and a pharmaceutically acceptable carrier. “Pharmaceutically acceptable carrier” can include any and all solvents, dispersion media, coatings, antibacterial and antifungal agents, isotonic and absorption delaying agents, and the like that are physiologically compatible. Specific examples may be one or more of water, saline, phosphate buffered saline, dextrose, glycerol, ethanol, etc., and combinations thereof. In many cases, isotonic agents such as sugars, polyols (e.g., mannitol, sorbitol) or sodium chloride, etc. are included in the pharmaceutical compositions. Of course, the pharmaceutically acceptable carrier may also include minor amounts of auxiliary substances, such as wetting or emulsifying agents, preservatives or buffering agents, to prolong the shelf life or potency of the antibody.

For example, the antibody of the invention can be incorporated into pharmaceutical compositions suitable for parenteral administration (e.g., intravenous, subcutaneous, intraperitoneal, intramuscular). These pharmaceutical compositions can be prepared in various forms. For example, liquid, semisolid, and solid dosage form, and the like, include, but are not limited to, liquid solution (e.g., injection solution and infusion solution), dispersion or suspension, tablet, pill, powder, liposome and suppository. Typical pharmaceutical compositions are in the form of injection solution or infusion solution. The antibody can be administered by intravenous infusion or injection or intramuscular or subcutaneous injection.

Of course, the anti-IL-11 antibody herein can also be made into a kit or part of other diagnostic reagents as needed. According to the embodiments of the present invention, the present invention also provides a kit, which comprises the above-mentioned IL-11 antibody. The application of the kit provided by the present invention, for example, the kit can be used for immunoblotting, immunoprecipitation, etc., involving the use of the specific binding properties of IL-11 antigen and antibody for detection, and the like. These kits may contain any one or more of the following: antagonist, anti-IL-11 antibody, or drug reference material; protein purification column; immunoglobulin affinity purification buffer; assay diluent for cells; instruction or literature, etc. Anti-IL-11 antibody can be used in different types of diagnostic tests, such as in vitro or in vivo detection of various diseases or the presence of drugs, toxins, or other proteins. For example, it can be used to test for related diseases by testing the serum or blood of the subject. Such related diseases may include IL-11 related diseases, such as pulmonary fibrosis and the like. Of course, the antibody provided herein can also be used for radioimmunoassay and radioimmunotherapy of the above-mentioned diseases.

When using the anti-IL-11 antibody provided by the present invention to treat the above-mentioned diseases, the anti-IL-11 antibody provided by the present invention may be provided to the subject. To this end, the present invention provides a method for treating the above-mentioned diseases, comprising administering the antibody or antigen-binding fragment thereof provided by the present invention to a subject in need.

The scheme of the present invention will be explained below in conjunction with the embodiments. Those skilled in the art will understand that the following examples are only used to illustrate the present invention, and should not be construed as limiting the scope of the present invention. If no specific technique or condition is indicated in the examples, the technique or condition described in the literature in the field or the product instruction is used. The reagents or instruments used without the manufacturer's indication are conventional products that can be obtained from the market.

### Example 1 Production of Recombinant Human Protein Antigen Hu-IL-11-His Tag

Construction of vector: The amino acid sequence of human IL-11 was obtained from the global public database UniProt (P20809: 124294). An expression vector containing human IL-11 gene sequence was constructed using the method of molecular cloning. The vector encoding human IL-11 was transfected into mammalian cells CHO through electrotransfection for expression for 14 days. After fermentation, the cell supernatant was harvested, and Ni column (GE healthcare, Cat: 17524801) was used for preliminary purification, then Superdex75 (GE healthcare, Cat: 29148721) was used for refine purification, finally the recombinant protein antigen hu-IL-11-His tag that can be used to immunize animals was obtained.

### Example 2 Animal Immunization

In order to obtain the mice that can secret antibody against hu-IL-11, we immunized female Balb/c mice. The female Balb/c mice were purchased from Hunan SJA Laboratory Animal Co., Ltd. The recombinant protein antigen hu-IL-11-His tag and immune adjuvant were fully emulsified before animal immunization. The immunization process is shown in Table 1.

TABLE 1
Schedule of Animal immunization

| Immunization | | |
|---|---|---|
| time point | | |
| (Day) | Immunogen dose | Immune mode |
| 0 | 50 ug hu-IL-11-His tag and | Intraperitoneal multipoint |
| | CFA mixed in equal volume | injection |
| 14 | 50 ug hu-IL-11-His tag and | Intraperitoneal multipoint |
| | IFA mixed in equal volume | injection |
| 28 | 50 ug hu-IL-11-His tag and | Intraperitoneal multipoint |
| | IFA mixed in equal volume | injection |
| 35 | None | Orbital blood collection |
| 38 | 50 ug hu-IL-11-His tag | Tail vein injection |
| 41 | None | The mice were sacrificed |
| | | and the spleens were |
| | | removed |

On the 35th day, a small amount of mice orbital blood was harvested, and the antibody titer was detected by the conventional Elisa method.

### Example 3 Fusion and Screening of Antibody-Secreting Cells

Isolated mouse spleen cells were fused with immortalized mouse myeloma cells SP2/0, then stood and cultured in RPMI-1640 complete medium containing 10⁻⁴ M Hypoxanthine, 4×10⁻⁷ M Aminopterin, 1.6×10⁻⁵ M Thymidine for about 14 days, the antibody affinity detection and screening were carried out after the fusion cells grew clone group. The positive clones that bound to IL-11 were selected, and the supernatants were taken, then flow cytometry was performed to determine the competitive binding of antigen and antibody. Cell lines with high secretion of specific antibody were selected for expanded culture and cryopreservation. The results are shown in Table 2. The specific method was to obtain an antibody strain with high affinity (OD450 nm was the measured affinity value) for IL-11 through Elisa screening firstly, wherein P.C. was the binding affinity between the purchased IL-11 antibody (Abcam, ab130083) and IL-11 protein as a positive control. Secondly, by constructing a cell line stably expressing IL-11R, an appropriate amount of IL-11 protein was mixed with the positive hybridoma supernatant, then co-incubated with a cell line stably expressing IL-11R, the blocking effect (mean value of FITC-A) of hybridoma supernatant on IL-11/IL-11R was finally detected by flow cytometry, wherein N.C. was the value detected without hybridoma supernatant, which could be regarded as the value without blocking at all (Negative control). The underline in Table 2 indicates the selected clones. After multiple rounds of subcloning, a monoclonal antibody derived from a single antibody-secreting cell was finally obtained. The binding affinity (OD450 nm) and the competitive blocking results of the monoclonal antibodies are shown in Table 3.

TABLE 2
Test results of Elisa affinity test and flow cytometry competition test

| | OD(450 nm) | FITC-A |
|---|---|---|
| Clone(No.) | value | mean |
| 3-1A2 | 2.243 | 12538 |
| 3-2A12 | 1.781 | 13334 |
| 3-2B2 | 1.084 | 13285 |
| 3-2B5 | 3.432 | 13837 |
| 3-3B9 | 1.777 | 13049 |
| 3-3G9 | 3.078 | 11577 |
| 3-3G11 | 2.943 | 13959 |
| 3-4G12 | 2.991 | 14418 |
| 3-4H1 | 2.876 | 12658 |
| 3-4H4 | 2.736 | 14459 |
| 3-4H12 | 3.33 | 13876 |
| 3-5B2 | 3.264 | 11001 |
| 3-5D11 | 1.068 | 13000 |
| 3-5G12 | 1.907 | 14267 |
| 3-6A4 | 2.656 | 750 |
| 3-7C1 | 2.661 | 13349 |
| 3-7C6 | 2.589 | 11605 |
| 3-7C9 | 2.722 | 11406 |
| 3-7G12 | 2.959 | 12412 |
| 3-7H5 | 1.995 | 13161 |
| 3-8H12 | 2.926 | 13360 |
| 3-9C12 | 1.915 | 14499 |
| 3-9D12 | 2.732 | 13833 |
| 3-9E1 | 2.894 | 13822 |
| 3-12F10 | 0.628 | 17067 |
| 3-13F11 | 1.915 | 15967 |
| 3-13G12 | 2.732 | 15312 |
| 3-13H1 | 1.41 | 13551 |
| 3-13H8 | 2.066 | 14323 |
| 3-15A11 | 1.214 | 16661 |
| 3-15C4 | 1.318 | 15017 |
| 3-15D5 | 2.894 | 11919 |
| 3-15D10 | 2.402 | 15064 |
| 3-17D12 | 2.48 | 15572 |
| 3-17E7 | 2.372 | 13301 |
| 3-18C11 | 2.566 | 14884 |
| 3-18C12 | 3.504 | 16114 |
| 3-19E8 | 3.412 | 15279 |
| 3-20F12 | 3.496 | 14504 |
| 5-1A2 | 2.765 | 6217 |
| 5-1B12 | 2.865 | 4613 |
| 5-1E9 | 2.531 | 4638 |
| 5-2E11 | 2.921 | 5218 |
| 5-2F11 | 2.275 | 5625 |
| 5-2G11 | 2.948 | 5045 |
| 5-3E8 | 2.775 | 4897 |
| 5-3G12 | 2.77 | 8573 |
| 5-4C9 | 2.034 | 5933 |
| 5-5F5 | 2.768 | 4564 |
| 5-5F8 | 2.532 | 9436 |
| 5-5F11 | 2.627 | 12273 |
| 5-6F1 | 2.677 | 11496 |
| 5-6G5 | 2.679 | 10201 |
| 5-7B4 | 2.751 | 6587 |
| 5-9E12 | 2.049 | 10707 |
| 5-9D7 | 2.129 | 10127 |
| 5-9G8 | 3.591 | 6747 |
| 5-8G1 | 3.714 | 10460 |
| 5-10D3 | 3.201 | 10041 |
| 5-10F6 | 2.311 | 10472 |
| 5-12B1 | 2.351 | 9782 |
| 5-14B10 | 2.54 | 11385 |
| 5-14C3 | 3.654 | 7672 |
| 5-17C11 | 3.462 | 11718 |
| 5-20E11 | 3.649 | 4330 |
| 5-21G2 | 3.545 | 4946 |
| 5-21H7 | 3.25 | 11385 |
| 5-22C4 | 3.561 | 6019 |
| 5-24F6 | 3.571 | 9954 |
| 5-25D3 | 1.87 | 3713 |
| P.C. (Elisa) | 3.62 | N/A |
| N.C. (Flow) | N/A | 24596 |

Note:
N/A in Table 2 means: no data

TABLE 3
Test results of Elisa affinity test
and flow cytometry competition test

| Sample | | FITC | Sample | | FITC |
|---|---|---|---|---|---|
| number | OD450 | Mean | number | OD450 | Mean |
| 3-6A4 | 1.584 | 3036 | 3-5B2-2G4 | 1.65 | 19751 |
| 5-20E11-5F2 | 1.596 | 3109 | 5-21G12-1D3 | 1.528 | 3362 |
| 5-20E11-1A2 | 1.634 | 3635 | 3-5B2-7A4 | 1.636 | 20958 |
| 3-15D5-3A6 | 1.688 | 10944 | 3-9E1-3A2 | 1.544 | 9067 |
| 3-7C9-1A6 | 1.601 | 3148 | 3-15D5-6A1 | 1.511 | 13639 |
| 3-2B5-3D2 | 1.614 | 15289 | N.C.(flow) | N/A | 18433 |

Note:
N/A in Table 3 means: no data; because the sequence of 3-6A4 cells after subcloning was consistent with the sequence before subcloning, its number is the same as that in Table 2.

### Example 4 Sequence Determination of Monoclonal Antibodies

1*10\hat{ }6˜5*10\hat{ }6 hybridoma cells were taken, the total RNA was extracted with Takara MiniBEST Universal RNA Extraction Kit, and cDNA was reverse transcribed with PrimeScript RT Reagent Kit (Takara Corporation). The above steps were carried out according to the instructions provided by the manufacturer. The design of gene amplification primers for antibody heavy chain variable region (VH) and light chain variable region (VL) mainly refers to the primer pairs in Ig-Primer Sets (Novagen Corporation, Cat. No. 69831-3), synthesized by BGI Genomics. The genes of heavy chain variable region and light chain variable region were amplified from the first strand of the cDNA, then cloned into the pMD18-T vector and transformed into E. coli DH5a, the clones were picked and sequenced by BGI Genomics to obtain the gene sequences of the heavy chain variable region (VH) and light chain variable region (VL). The primer sequences used and the sequenced antibody sequences are also found in the documentation.

### Example 5 Identification of Antibody Isotypes

The monoclonal hybridoma cell lines corresponding to the preferred antibody strains were injected into Balb/C mice by conventional method to produce and purify ascites antibodies. The purified antibody was used for antibody isotype analysis, and the antibody isotype detection method was detected by a mature kit (Proteintech, KMIM-2). The results are shown in Table 4 below.

TABLE 4
Antibody isotype identification results of candidate antibody strains

| Clones | | | | | | | | | | | |
|---|---|---|---|---|---|---|---|---|---|---|---|
| OD | 3-5B2- | 3-5B2- | 3-2B5- | | 3-7C9-1 | 3-9E1-3 | 3-15D | 3-15D | 5-20E1 | 5-21G | 5-20E1 |
| Isotype | 2G4 | 7A4 | 3D2 | 3-6A4 | A6 | A2 | 5-3A6 | 5-6A1 | 1-1A2 | 2-1D3 | 1-5F2 |
| IgG1 | 0.307 | 1.207 | 0.113 | 3.098 | 0.475 | 0.501 | 0.241 | 0.402 | 1.853 | 1.231 | 2.457 |
| IgG2a | 0.055 | 0.071 | 0.063 | 0.095 | 0.081 | 0.072 | 0.096 | 0.074 | 0.265 | 0.075 | 0.293 |
| IgG2b | 0.051 | 0.307 | 0.823 | 0.071 | 0.086 | 0.075 | 0.474 | 0.075 | 0.088 | 0.053 | 0.285 |
| IgG3 | 0.091 | 0.091 | 0.057 | 0.057 | 0.106 | 0.074 | 0.097 | 0.065 | 0.177 | 0.055 | 0.155 |

### Example 6 Expression and Purification of Preferred Monoclonal Antibodies

The sequences of preferred monoclonal antibodies 3-6A4 and 5-20E11-5F2 were constructed into conventional expression vectors by genetic engineering method, and then transfected into CHO cells. After fermentation, the collected cell culture supernatants were purified by a Protein G column (EzFast Protein G 4FF, Borgron), the balance buffer was 20 mM PBS, 0.15M NaCl, pH 7.4, and 0.1M glycine, pH 3 0.0±0.2 was used as the elution buffer. The protein eluate under the target absorption peak was collected, and after ultrafiltration with PBS buffer, part of the samples were taken for SDS-PAGE electrophoresis detection, and the aggregation content was determined by SEC-HPLC method. The aggregation contents of the antibodies were all within 3%.

### Example 7 Preferred Antibodies Competitively Block the Binding of IL-11 to IL-11R

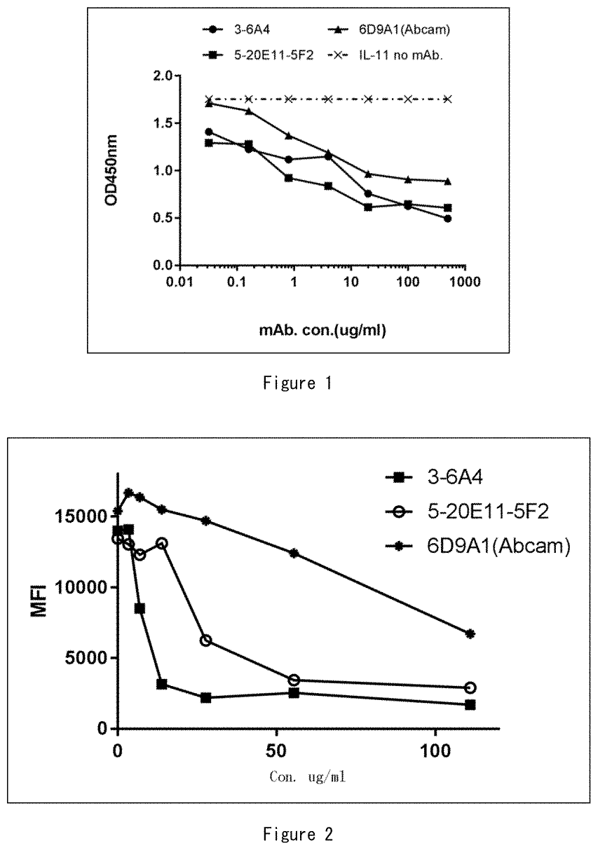

In order to detect the competitive binding ability of the preferred antibodies 3-6A4 and 5-20E11-5F2, the Elisa plates were coated with an appropriate amount of IL-11R-His protein and incubated overnight. Then, an appropriate amount of IL-11-Fc protein was added, and co-incubated with different concentration gradients of antibodies. Finally, HRP-conjugated Goat anti IgG-Fc secondary antibody (ThermoFisher, 31413) and TMB substrate were used for detection. We detected that both the preferred antibodies and 6D9A1 (Abcam, ab130083) could inhibit the binding of IL-11 to IL-11R to varying degrees. The results are shown in FIG. 1.

### Example 8 Cross-Reactivity of Preferred Antibodies

IL-6 cytokine family members include IL-6, IL-11, CNTF, CLC, LIF, CT-1, OSM, IL-27, IL-31. IL-11 is a member of the IL-6 cytokine family. We used conventional Elisa method to coat recombinant IL-6 (Sinobiological-10395-HNAE), IL-27 (R&D systems, 2526-IL-010), LIF (Peprotech, 300-05), CLC (R&D systems, 962-CL-050), IL-11 (LSBio, LS-G132887-10), OSM (Genscript-z03132), IL-31 (Peprotech, 200-31), CNTF (R&D systems, 257-NT-010), CT-1 (R&D systems, 612-CD-010) protein, and the binding affinity of preferred antibodies 3-6A4 and 5-20E11-5F2 to members of the IL-6 cytokine family was detected. The results show that the preferred antibodies do not cross-react with other members of the IL-6 cytokine family except for binding to the IL-11 cytokine. The Elisa results (OD450 nm) are shown in Table 5 below.

TABLE 5
Binding affinity detection of preferred antibodies 3-6A4 and 5-20E11-5F2
to different members of the IL-6 cytokine family

| | IL-6 | IL-27 | OSM | CLC | LIF | IL-31 | CNTF | CT-1 | IL-11 |
|---|---|---|---|---|---|---|---|---|---|
| 3-6A4 | 0.039 | 0.035 | 0.102 | 0.042 | 0.084 | 0.088 | 0.064 | 0.041 | 1.884 |
| 5-20E11-5F2 | 0.049 | 0.048 | 0.045 | 0.033 | 0.037 | 0.036 | 0.036 | 0.034 | 1.926 |

### Example 9 Binding Affinity Determination of Preferred IL-11 Monoclonal Antibodies to Antigen

Bio-Layer Interferometry (BLI) was used for binding affinity determination. Ni-NTA sensor was used to capture antigen IL-11 (His tag), the preferred antibodies 3-6A4 and 5-20E11-5F2 were used as analytes. The analytes were diluted in 7 concentration gradients, the binding time was 60 s and the dissociation time was 1000 s. The buffer was 20 mM PBS, 0.15M NaCl, 0.1% BSA, 0.05% Tween 20, pH 7. BLI kinetics/affinity software was used to analysis and obtain the dissociation constant (kdis) and affinity constant (KD) of the preferred antibodies and the corresponding hu-IL-11 ligand. The specific results are shown in Table 6 below.

TABLE 6

| | Conc. | | | | | | | | |
|---|---|---|---|---|---|---|---|---|---|
| Sample ID | (nM) | Response | KD (M) | kon (1/Ms) | kon Error | kdis (1/s) | kdis Error | Full X\hat{ }2 | Full R\hat{ }2 |
| 3-6A4 | 41.7 | 1.0442 | 3.93E−11 | 1.06E+06 | 2.08E+04 | 4.16E−05 | 2.39E−06 | 0.017992 | 0.999488 |
| 5-20E11-5F2 | 166.7 | 1.2727 | 6.90E−11 | 1.75E+05 | 3.19E+03 | 1.21E−05 | 2.94E−06 | 0.051363 | 0.999171 |

### Example 10 Competitive Blocking Activity Detection of Preferred Antibodies

In order to evaluate the binding of the preferred Anti-huIL-11 antibodies 3-6A4 and 5-20E11-5F2 to IL-11 and whether the binding between the ligand and the receptor can be blocked, we constructed a cell line (membrane-IL-11R CHO) that stably expressing IL-11R. The blocking activities of candidate antibodies and commercial antibody 6D9A1 (Abcam, ab130083) against IL-11 and IL-11R were detected by flow cytometry. Firstly, an appropriate amount of IL-11 recombinant protein with Fc tag was co-incubated with different gradients of antibodies for 15 mins, and then incubated with 1 E+05 membrane-IL-11R CHO cells. The secondary antibody used in the detection was a secondary antibody conjugated with a FITC fluorophore (Goat-anti-Fc-FITC) (Abcam, ab98529). The competitive binding results show that the neutralizing activity IC50 of the 3-6A4 antibody is about 7 μg/ml; the neutralizing activity IC50 of the 5-20E11-5F2 antibody is about 21 μg/ml, and the blocking activity of the candidate antibodies is significantly better than that of the 6D9A1 clone antibody, the results are shown in FIG. 2.

### Example 11 Preferred Antibodies Inhibit the Transformation of Lung Fibroblasts HFL-1 into Fibrosis

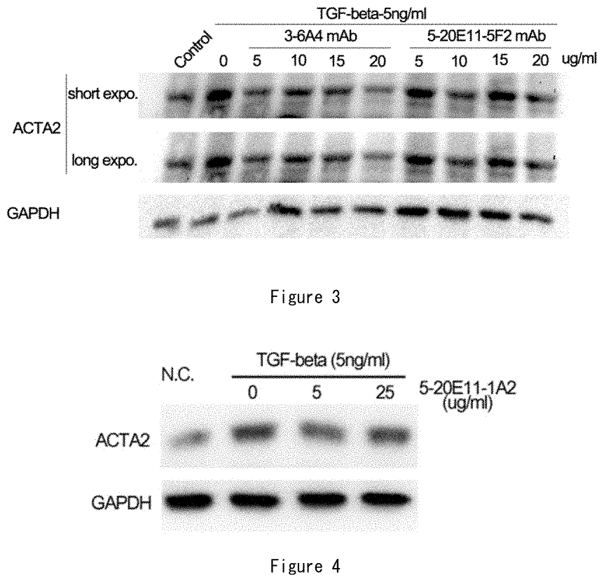

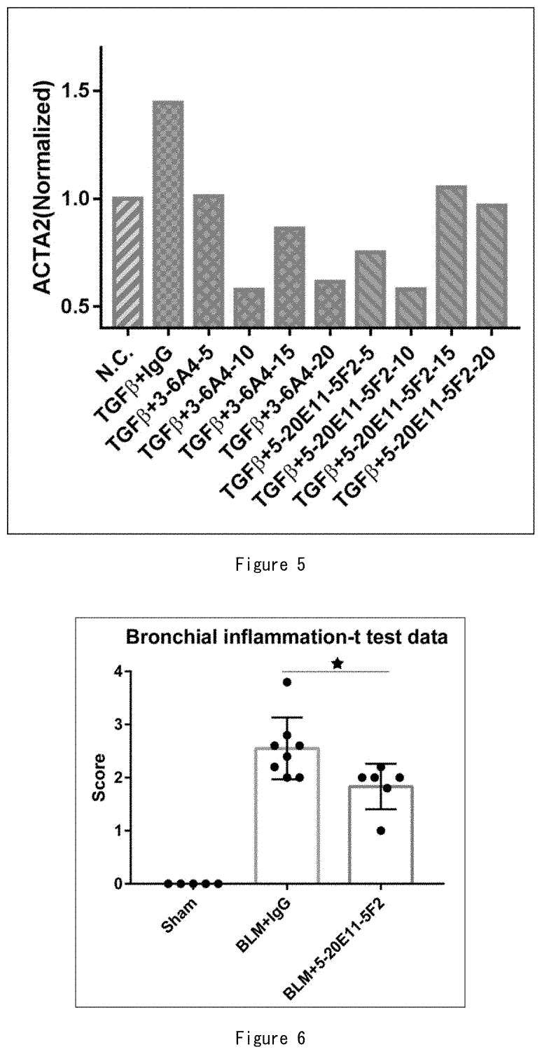

As lung fibroblasts, HFL-1 cells were induced by pro-fibrotic factors such as TGF-beta1 and expressed a large amount of IL-11 cytokines, and finally transformed into fibrosis. When HFL-1 cells were transformed into fibrosis, the marker ACTA2 protein was accumulated continuously. In this example, when TGF-beta1 cytokine was used to induce HFL-1 cells to transform into fibrosis, the preferred antibodies 3-6-A4, 5-20E11-1A2 and 5-20E11-5F2 were added at the same time. After culturing for 48 h, the cells were harvested for western blot detection analysis. It can be seen that different concentrations of antibodies can inhibit the transformation of HFL-1 cells to fibrosis (the expression of ACTA2 protein was reduced), as shown in FIG. 3, FIG. 4 and the corresponding statistical analysis FIG. 5.

### Example 12 Preferred Antibodies Inhibit Pulmonary Fibrosis in C57BL/6 Mice

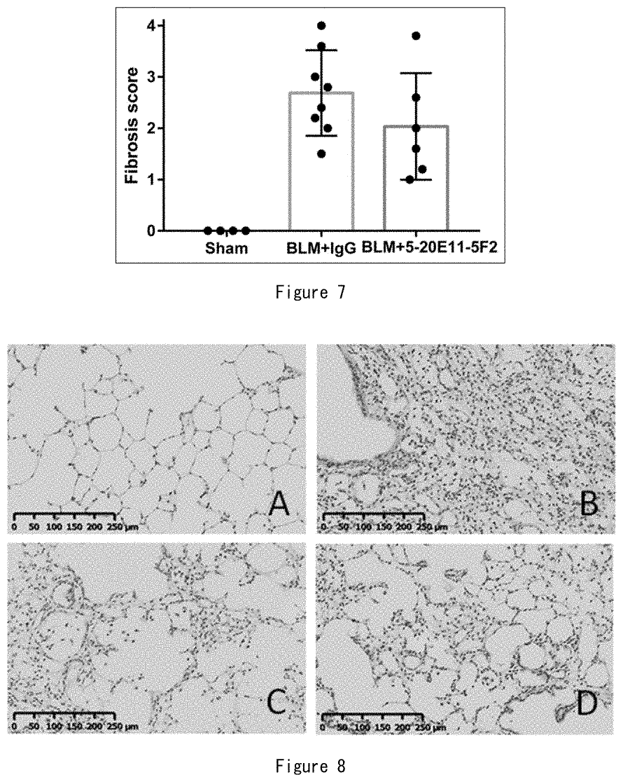

In an animal model of pulmonary fibrosis in C57BL/6 mice induced by bleomycin, specifically, 10-week-old C57BL/6 was subjected to general anesthesia with pentobarbital anesthetic and the main trachea of the mouse lung was surgically exposed. Then an appropriate amount of bleomycin was injected into the trachea by instillation or nebulization. After seven days of bleomycin induction in mice, pulmonary fibrosis was basically appeared. On the eighth day, the preferred antibody 5-20E11-5F2 or the isotype control antibody IgG was injected every other day. After 14 days of continuous injection, the mice were sacrificed and the lungs were removed, and the inflammatory damage and fibrosis scores of the lungs were detected. Under this pharmacodynamic model, it can be seen that the candidate antibodies can slow down pulmonary fibrosis in mice, and the results are shown in FIGS. 6 and 7.

### Example 13 Preferred Antibodies Inhibit Pulmonary Fibrosis in Sprague Dawley Rats

Intratracheal instillation of bleomycin in rats was a widely used pharmacodynamic model to study pulmonary fibrosis, the ability of the preferred antibodies to reduce bleomycin-induced unilateral pulmonary fibrosis in rats in vivo was tested in this experiment.

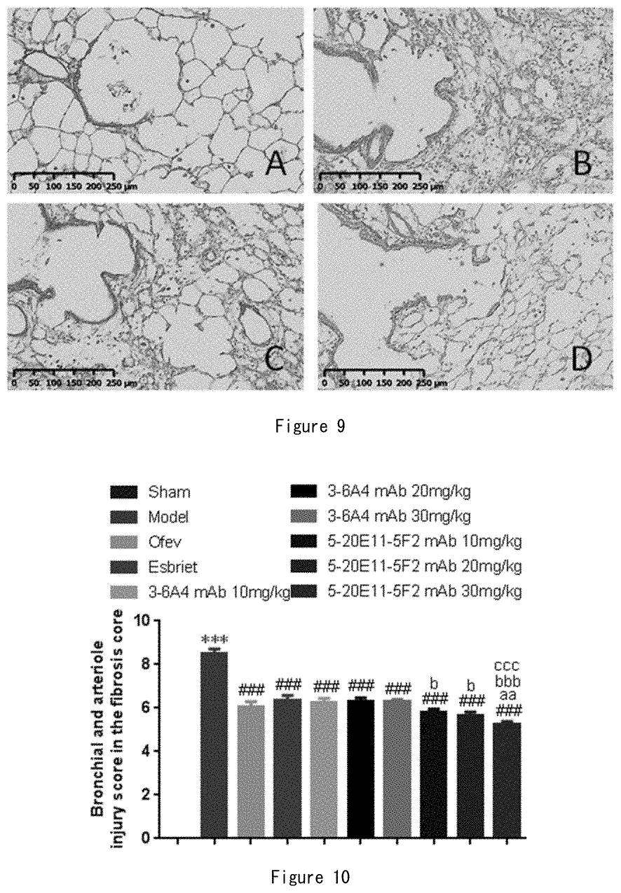

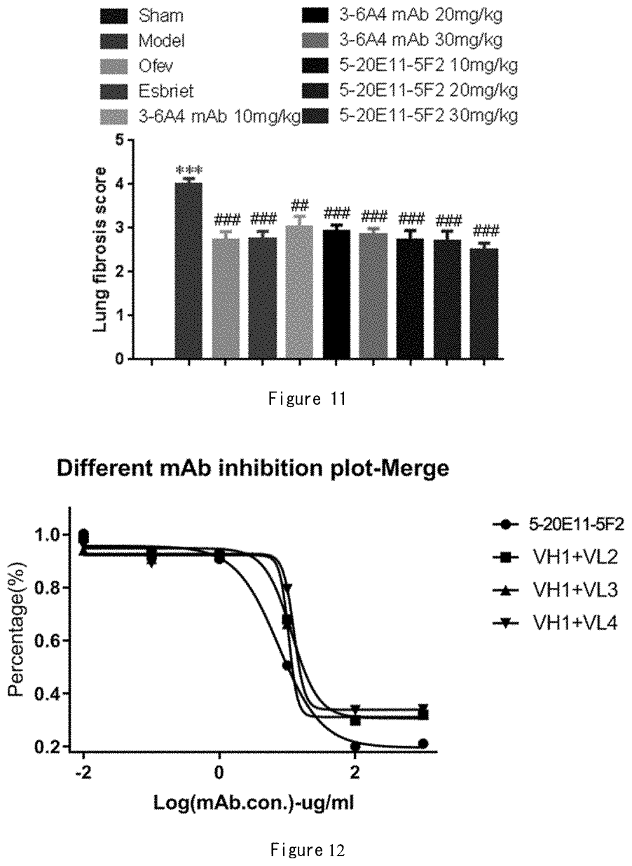

The male animals were divided into nine groups. The depth of anesthesia was maintained by 25 mg/kg pentobarbital sodium combined with 1-2.5% isoflurane respiratory anesthesia. The fibrosis groups were induced by tracheal injection of bleomycin, and in sham-operated group, the bleomycin was replaced by an equal volume of saline. On the eighth day of model establishment, different groups were treated with different doses of the preferred antibody for 14 days every other day, wherein pirfenidone (Esbriet) or nintedanib (Ofev) were used as the treatment reference (Esbriet and Ofev were both used 100 mg/kg/day). After the treatment, lungs were taken for immunohistochemical H&E staining and Masson's tri-staining. The histological examination of the rat lungs showed that the control group was normal. In the bleomycin-treated lungs, the inflammatory damage was significantly aggravated, and the preferred antibody could significantly alleviate the infiltration of inflammatory cells in the lungs, as shown in FIG. 8 (FIG. 8, H&E staining of lung tissue, A is the control group; B is the bleomycin-induced group; C is the 3-6A4 mAb-20 mg/kg group; D is the 5-20E11-5F2 mAb-20 mg/kg group). In addition, the preferred antibodies could also greatly alleviate the process of pulmonary fibrosis, as shown in FIG. 9 (FIG. 9, Masson's tri-staining of lung tissue, A is the control group; B is the bleomycin-induced group; C is the 3-6A4 mAb-20 mg/kg group; D is 5-20E11-5F2 mAb-20 mg/kg). The statistical analysis results show that the preferred antibodies can alleviate the inflammatory damage and fibrosis process of rat lung caused by bleomycin at different doses, as shown in FIG. 10 (FIG. 10, ###P<0.001 vs. BLM Model; aaP<0.01 vs. Ofev; bP<0.05 vs. Esbriet, bbbP<0.001 vs. Esbriet; cccP<0.001 vs. 3-6A4 mAb 30 mg/kg), FIG. 11 (***P<0.001 vs. Sham; ##P<0.01 vs. BLM Model, ###P<0.001 vs. BLM Model.).

### Example 14 Humanization of IL-11 Antibody

The humanization of the IL-11 antibody in the above examples was carried out by complementarity determining region grafting (CDR grafting). Firstly, the human germline antibody sequence with the highest homology to the light and heavy chain variable regions of murine antibody was found. The germline selected for humanization of the heavy chain variable region of the antibody was IGHV1-46*01, IGKV3-11*01 was selected for humanization of the light chain variable region. The CDR region of the murine antibody was retained, and the framework sequence of the murine antibody was replaced with the framework sequence of the human germline antibody. Secondly, the structure model of the murine antibody Fv fragment was established, and BLAST was performed in the PDB antibody database, and the structure INQB was selected as the template for homology modeling. Each different amino acid position in the structural model of human antibody and the corresponding murine antibody was compared one by one, the amino acid that did not meet the FR structural specification, the amino acid located in the FR core, and the amino acid located at the VH-VL interface were selected and backmutated to murine sequences.

The sequences of the obtained heavy chain variable region and the light chain variable region of the IL-11 humanized antibody are shown in SEQ ID NO: 143-146 and SEQ ID NO: 147-150.

Sequence of IL-11 humanized antibody heavy chain
variable region (VH1):
(SEQ ID NO: 143)
QVQLVQSGAEVKKPGASVKVSCKASDYTFTNYWLGWVRQAPGQGLEWMGH
IYPGGGYTNYNEKFKGRVTMTRDTSTSTVYMELSSLRSEDTAVYYCARVR
SGNDALDFWGQGTLVTVSS.
Sequence of IL-11 humanized antibody heavy chain
variable region (VH2):
(SEQ ID NO: 144)
QVQLVQSGAEVKKPGASVKVSCKASDYTFTNYWLGWVRQAPGQGLEWIGH
IYPGGGYTNYNEKFKGRVTMTADTSTSTVYMELSSLRSEDTAVYFCARVR
SGNDALDFWGQGTLVTVSS.
Sequence of IL-11 humanized antibody heavy chain
variable region (VH3):
(SEQ ID NO: 145)
QVQLVQSGAEVKKPGASVKVSCKASDYTFTNYWLGWVRQAPGQGLEWIGH
IYPGGGYTNYNEKFKGRATLTADTSTSTVYMELSSLRSEDTAVYFCARVR
SGNDALDFWGQGTLVTVSS.
Sequence of IL-11 humanized antibody heavy chain
variable region (VH4):
(SEQ ID NO: 146)
QVQLVQSGAEVKKPGASVKVSCKASDYTFTNYWLGWVRQAPGHGLEWIGH
IYPGGGYTNYNEKFKGRATLTADTSTSTVYMELSSLRSEDTAVYFCARVR
SGNDALDFWGQGTLVTVSS.
Sequence of IL-11 humanized antibody light chain
variable region (VL1):
(SEQ ID NO: 147)
EIVLTQSPATLSLSPGERATLSCRASESVDEYGISFMNWYQQKPGQAPRL
LIYSASNQGSGIPARFSGSGSGTDFTLTISSLEPEDFAVYYCQQSKEVPW
TFGGGTKLEIK.
Sequence of IL-11 humanized antibody light chain
variable region (VL2):
(SEQ ID NO: 48)
EIVLTQSPATLSLSPGERATLSCRASESVDEYGISFMNWYQQKPGQAPRL
LIYSASNQGSGIPARFSGSGSGTDFTLTISSLEPEDFAVYFCQQSKEVPW
TFGGGTKLEIK.
Sequence of IL-11 humanized antibody light chain
variable region (VL3):
(SEQ ID NO: 149)
EIVLTQSPATLSLSPGERATLSCRASESVDEYGISFMNWFQQKPGQAPRL
LIYSASNQGSGIPARFSGSGSGTDFTLTISSLEPEDFAVYFCQQSKEVPW
TFGGGTKLEIK.
Sequence of IL-11 humanized antibody light chain
variable region (VL4):
(SEQ ID NO: 150)
EIVLTQSPATLSLSPGERATLSCRASESVDEYGISFMNWFQQKPGQAPRL
LIYSASNQGSGIPARFSGSGSGTDFTLTISSLEPEDFAMYFCQQSKEVPW
TFGGGTKLEIK.

The nucleic acid sequences encoding the light chain and heavy chain of the IL-11 humanized antibody were synthesized and inserted into the eukaryotic expression vector pcDNA3.4. 4 heavy chain sequence vectors were paired respectively with 4 light chain sequence vectors and co-transfected into mammalian cells CHO for expression to obtain a single-chain antibody in the form of scFv. One week later, the supernatant containing the scFv antibodies were collected for affinity detection.

### Example 15 Affinity Ranking of IL-11 Humanized Antibody

Biacore was used to measure the binding affinity of each humanized antibody supernatant scFv to IL-11, and sorted according to the dissociation constant kd (1/s), and three humanized antibodies with the smallest dissociation constant kd (1/s) were selected. The measure method included the steps of capturing the humanized antibody at a flow rate of 10 ul/min. The flow rate was switched to 30 ul/min, 400 nM of IL-11 antigen was flowed through the sample channel, the binding time was 180 s, and the dissociation time was 600 s. Finally the chip was regenerated with 10 mM glycine buffer. The results are shown in Table 7. The humanized antibodies VH1+VL2, VH1+VL3, VH1+VL4 dissociated from IL-11 at a slower rate than other humanized antibodies.

TABLE 7
Affinity of IL-11 Humanized Antibodies to IL-11 Antigen

| | | | | | Rmax | |
|---|---|---|---|---|---|---|
| Ligand | Analyte | ka (1/Ms) | kd (1/s) | KD (M) | (RU) | Chi² (RU²) |
| BLANK(PBS) | IL-11 | NA | NA | NA | NA | NA |
| 5-20E11-5F2 | IL-11 | 5.84E+04 | 1.22E−04 | 2.09E−09 | 213.2 | 1.14E−01 |
| NC(culture | IL-11 | NA | NA | NA | NA | NA |
| medium) | | | | | | |
| VH1 + VL1-scFv | IL-11 | 3.51E+04 | 7.58E−04 | 2.16E−08 | 257.1 | 8.24E−01 |
| VH1 + VL2-scFv | IL-11 | 4.13E+04 | 4.61E−04 | 1.12E−08 | 247.2 | 5.66E−01 |
| VH1 + VL3-scFv | IL-11 | 3.55E+04 | 4.36E−04 | 1.23E−08 | 239.7 | 4.35E−01 |
| VH1 + VL4-scFv | IL-11 | 3.31E+04 | 3.87E−04 | 1.17E−08 | 268.7 | 3.92E−01 |
| VH2 + VL1-scFv | IL-11 | 5.34E+04 | 8.90E−04 | 1.67E−08 | 142.3 | 7.35E−01 |
| VH2 + VL2-scFv | IL-11 | 4.57E+04 | 8.12E−04 | 1.78E−08 | 208.9 | 7.67E−01 |
| VH2 + VL3-scFv | IL-11 | 4.56E+04 | 5.41E−04 | 1.19E−08 | 185.7 | 4.06E−01 |
| VH2 + VL4-scFv | IL-11 | 4.88E+04 | 5.28E−04 | 1.08E−08 | 174.5 | 4.64E−01 |
| VH3 + VL1-scFv | IL-11 | 4.45E+04 | 1.00E−03 | 2.25E−08 | 184.8 | 8.66E−01 |
| VH3 + VL2-scFv | IL-11 | 4.65E+04 | 6.80E−04 | 1.46E−08 | 184.4 | 4.71E−01 |
| VH3 + VL3-scFv | IL-11 | 4.76E+04 | 4.64E−04 | 9.76E−09 | 181.4 | 4.14E−01 |
| VH3 + VL4-scFv | IL-11 | 4.18E+04 | 5.56E−04 | 1.33E−08 | 219.5 | 5.76E−01 |
| VH4 + VL1-scFv | IL-11 | 4.89E+04 | 8.40E−04 | 1.72E−08 | 128.4 | 4.84E−01 |
| VH4 + VL2-scFv | IL-11 | 5.52E+04 | 6.14E−04 | 1.11E−08 | 135.9 | 4.22E−01 |
| VH4 + VL3-scFv | IL-11 | 4.61E+04 | 5.16E−04 | 1.12E−08 | 181.7 | 3.90E−01 |
| VH4 + VL4-scFv | IL-11 | 4.79E+04 | 4.81E−04 | 1.00E−08 | 147.6 | 2.54E−01 |

### Example 16 Expression and Purification of Preferred IL-11 Humanized Antibodies

The preferred IL-11 humanized antibodies VH1+VL2, VH1+VL3, VH1+VL4 were respectively constructed with full-length sequences (including Fc part), and transfected into mammalian cells CHO for expression. After fermentation, the collected cell culture supernatants were purified by Protein A column, the balance buffer was 20 mM PBS, pH 7.4, and the eluent was 0.1M glycine pH 3.0±0.2, and the protein eluent under the target absorption peak was collected. After ultrafiltration with PBS buffer, part of the samples were taken to measure the concentration by HPLC and purity by SDS-PAGE electrophoresis detection.

### Example 17 Affinity Detection of Preferred IL-11 Humanized Antibodies

The binding affinity of each preferred humanized full-length (IgG format) antibody sample to IL-11 was determined by Biacore. The measure method included the steps of capturing the humanized antibody at a flow rate of 10 ul/min. The flow rate was switched to 30 ul/min, and different concentrations of IL-11 antigen (400 nM, 200 nM, 100 nM, 50 nM, 25 nM, 12.5 nM, 6.25 nM) were flowed through the sample channel and reference channel, the binding time was 180 s, and the dissociation time was 600 s. Finally the chip was regenerated with 10 mM glycine buffer. As shown in Table 8, the affinity of the humanized antibody VH1+VL3 was better than that of the other two humanized antibodies, and slightly lower than that of the 5-20E11-5F2 antibody.

TABLE 8
Affinity of preferred humanized antibodies to IL-11 antigen

| | | | | | Rmax | |
|---|---|---|---|---|---|---|
| Ligand | Analyte | ka (1/Ms) | kd (1/s) | KD (M) | (RU) | Chi² (RU²) |
| VH1 + VL2 | IL-11 | 4.98E+04 | 5.97E−04 | 1.20E−08 | 114.1 | 4.03E−01 |
| VH1 + VL3 | IL-11 | 5.26E+04 | 4.74E−04 | 9.03E−09 | 105.6 | 3.71E−01 |
| VH1 + VL4 | IL-11 | 5.43E+04 | 5.45E−04 | 1.00E−08 | 63.26 | 1.25E−01 |
| 5-20E11-5F2 | IL-11 | 6.76E+04 | 4.33E−04 | 6.41E−09 | 72.58 | 6.99E−02 |

### Example 18 Competitively Blocking the Binding of IL-11 to IL-11R with Preferred Humanized Antibodies

To assess whether the preferred humanized antibodies could block the binding of IL-11 to IL-11R, a cell line (membrane-IL-11R CHO) that stably expressing IL-11R was constructed. The measure method included the following steps: IL-11-Fc protein with a concentration of 100 μg/ml was taken and added to a 96-well U-shaped plate with 10 ul/well, and incubated with 90 ul of serial dilutions of preferred humanized antibodies (100 ug/ml, 10 ug/ml, 1 ug/ml, 0.1 ug/ml, 0.01 ug/ml, 0.001 ug/ml) at room temperature for 1 h. Then the plate was incubated with 1.5*10\hat{ }5 membrane-IL-11R CHO cells (100 ul/well) for 1 h at room temperature. After incubation, the plate was centrifuged at 1500 rpm for 5 min, washed three times with PBS, and 1:400 diluted flow secondary antibody Goat-anti-Fc-FITC (Abcam, ab98529) was added with 200 ul/well. Then, the plate was incubated at 4° C. for 1 h, centrifuged at 1500 rpm for 5 min, washed three times with PBS, resuspended cells in 180 ul PBS, and detected by flow cytometry. The results are plotted as an S-curve as shown in FIG. 12. The results show that the blocking activity of the humanized antibody was basically the same as that of the 5-20E11-5F2 antibody.

TABLE 9
EC50 of humanized antibody competitively
blocking the binding of IL-11 to IL-11R

| Antibody | | | | |
|---|---|---|---|---|
| number | 5-20E11-5F2 | VH1 + VL2 | VH1 + VL3 | VH1 + VL4 |
| EC50 (ug/ml) | 7.537 | 11.57 | 10.46 | 12.48 |

### Example 19 Preferred Humanized Antibodies Inhibit the Transformation of Hepatic Fibroblasts LX-2 to Fibrosis

LX-2 cells are human hepatic stellate cells, which can express a large amount of IL-11 cytokines when induced by pro-fibrotic factors such as TGF-beta1, and finally transform into fibrosis. When LX-2 cells transform into fibrosis, the marker ACTA2 protein will continue to accumulate. In this example, LX-2 cells were plated in a 6-well plate at a density of 3*10\hat{ }5 cells/well and cultured for 10 h until the cells adhered. After starvation in serum-free DMEM medium for 12 h, 5 ng/ml of TGF-beta1 cytokine was added for stimulation, and serial dilutions of preferred humanized antibodies (50 ug/ml, 25 ug/ml, 5 ug/ml) were added at the same time. After culturing for 24 h, the medium was taken, 200 ul of RIPA lysis solution (Biyuntian, P0013B) was added to each well for western blot analysis. The results show that different concentrations of humanized antibodies can inhibit the transformation of LX-2 cells into fibrosis (the expression of ACTA2 protein was decreased).

In the description of this specification, description with reference to the term “an embodiment,” “some embodiments,” “example,” “specific example,” or “some examples”, etc., means that a particular feature, structure, material, or characteristic described in connection with the embodiment or example is included in at least one embodiment or example of the present invention. In this specification, schematic representations of the above terms are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although the embodiments of the present invention have been shown and described above, it would be appreciated that the above-mentioned embodiments are exemplary and should not be construed to limit the present disclosure, changes, alternatives, and modifications to the above-described embodiments may be made by those of ordinary skill in the art within the scope of the present invention.

1.-28. (canceled)

29. An antibody or an antigen-binding fragment thereof that can specifically recognize IL-11, wherein the antibody comprises:
a heavy chain variable region comprising CDR1, CDR2, CDR3 as shown in SEQ ID NO:1, 2 and 3 or an amino acid sequence having at least 95% identity to SEQ ID NO:1, 2 and 3, respectively; or
a heavy chain variable region comprising CDR1, CDR2, CDR3 as shown in SEQ ID NO:4, 5 and 6 or an amino acid sequence having at least 95% identity to SEQ ID NO:4, 5 and 6, respectively; or
a heavy chain variable region comprising CDR1, CDR2, CDR3 as shown in SEQ ID NO:7, 8 and 9 or an amino acid sequence having at least 95% identity to SEQ ID NO:7, 8 and 9, respectively; or
a heavy chain variable region comprising CDR1, CDR2, CDR3 as shown in SEQ ID NO:10, 11 and 12 or an amino acid sequence having at least 95% identity to SEQ ID NO:10, 11 and 12, respectively; or
a heavy chain variable region comprising CDR1, CDR2, CDR3 as shown in SEQ ID NO:13, 14 and 15 or an amino acid sequence having at least 95% identity to SEQ ID NO:13, 14 and 15, respectively; or
a heavy chain variable region comprising CDR1, CDR2, CDR3 as shown in SEQ ID NO:16, 17 and 18 or an amino acid sequence having at least 95% identity to SEQ ID NO:16, 17 and 18, respectively; or
a heavy chain variable region comprising CDR1, CDR2, CDR3 as shown in SEQ ID NO:19, 20 and 21 or an amino acid sequence having at least 95% identity to SEQ ID NO:19, 20 and 21, respectively; or
a heavy chain variable region comprising CDR1, CDR2, CDR3 as shown in SEQ ID NO:22, 23 and 24 or an amino acid sequence having at least 95% identity to SEQ ID NO:22, 23 and 24, respectively; or
a heavy chain variable region comprising CDR1, CDR2, CDR3 as shown in SEQ ID NO:25, 26 and 27 or an amino acid sequence having at least 95% identity to SEQ ID NO:25, 26 and 27, respectively; or
a heavy chain variable region comprising CDR1, CDR2, CDR3 as shown in SEQ ID NO:28, 29 and 30 or an amino acid sequence having at least 95% identity to SEQ ID NO:28, 29 and 30, respectively; or
a heavy chain variable region comprising CDR1, CDR2, CDR3 as shown in SEQ ID NO:31, 32 and 33 or an amino acid sequence having at least 95% identity to SEQ ID NO: 31, 32 and 33, respectively; and
a light chain variable region comprising CDR1, CDR2, CDR3 as shown in SEQ ID NO:34, 35 and 36 or an amino acid sequence having at least 95% identity to SEQ ID NO:34, 35 and 36, respectively; or
a light chain variable region comprising CDR1, CDR2, CDR3 as shown in SEQ ID NO:37, 38 and 39 or an amino acid sequence having at least 95% identity to SEQ ID NO:37, 38 and 39, respectively; or
a light chain variable region comprising CDR1, CDR2, CDR3 as shown in SEQ ID NO:40, 41 and 42 or an amino acid sequence having at least 95% identity to SEQ ID NO:40, 41 and 42, respectively; or
a light chain variable region comprising CDR1, CDR2, CDR3 as shown in SEQ ID NO:43, 44 and 45 or an amino acid sequence having at least 95% identity to SEQ ID NO:43, 44 and 45, respectively; or
a light chain variable region comprising CDR1, CDR2, CDR3 as shown in SEQ ID NO:46, 47 and 48 or an amino acid sequence having at least 95% identity to SEQ ID NO:46, 47 and 48, respectively; or
a light chain variable region comprising CDR1, CDR2, CDR3 as shown in SEQ ID NO:49, 50 and 51 or an amino acid sequence having at least 95% identity to SEQ ID NO:49, 50 and 51, respectively; or
a light chain variable region comprising CDR1, CDR2, CDR3 as shown in SEQ ID NO:52, 53 and 54 or an amino acid sequence having at least 95% identity to SEQ ID NO:52, 53 and 54, respectively; or
a light chain variable region comprising CDR1, CDR2, CDR3 as shown in SEQ ID NO:55, 56 and 57 or an amino acid sequence having at least 95% identity to SEQ ID NO:55, 56 and 57, respectively; or
a light chain variable region comprising CDR1, CDR2, CDR3 as shown in SEQ ID NO:58, 59 and 60 or an amino acid sequence having at least 95% identity to SEQ ID NO:58, 59 and 60, respectively; or
a light chain variable region comprising CDR1, CDR2, CDR3 as shown in SEQ ID NO:61, 62 and 63 or an amino acid sequence at least 95% identity to SEQ ID NO: 61, 62 and 63; or
a light chain variable region comprising CDR1, CDR2, CDR3 as shown in SEQ ID NO:64, 65 and 66 or an amino acid sequence at least 95% identity to SEQ ID NO: 64, 65 and 66.

30. The antibody or antigen-binding fragment thereof of claim 29, wherein the heavy chain variable region has the amino acid sequence shown in any one of SEQ ID NO: 67-77.

31. The antibody or antigen-binding fragment thereof of claim 29, wherein the light chain variable region has the amino acid sequence shown in any one of SEQ ID NO: 78-88.

32. The antibody or antigen-binding fragment thereof of claim 29, wherein the antibody comprises at least one of a heavy chain constant region and a light chain constant region, and at least a part of at least one of the heavy chain constant region and the light chain constant region is derived from at least one of a murine antibody, a human antibody, a primate antibody, or a mutant thereof.

33. The antibody or antigen-binding fragment thereof of claim 29, wherein both the light chain constant region and the heavy chain constant region of the antibody are derived from a human IgG antibody or a mutant thereof.

34. The antibody or antigen-binding fragment thereof of claim 29, wherein the light chain constant region of the antibody is derived from the light chain constant region of human Kappa; the heavy chain constant region is derived from the heavy chain constant region of human IgG1.

35. The antibody or antigen-binding fragment thereof of claim 34, wherein the full-length sequence of the antibody constant region is shown in SEQ ID NO: 89 or 90.

36. The antibody or antigen-binding fragment thereof of claim 29, wherein the heavy chain has the amino acid sequence shown in any one of SEQ ID NO: 91-101 and the light chain has the amino acid sequence shown in any one of SEQ ID NO: 102-112.

37. The antibody or antigen-binding fragment thereof of claim 29, wherein the antibody is a single-chain antibody, a multimeric antibody, a CDR-grafted antibody or a small molecule antibody.

38. The antibody or antigen-binding fragment thereof of claim 37, wherein the single-chain antibody comprises a heavy chain variable region with the amino acid sequence shown in any one of SEQ ID NO: 67-77 and a light chain variable region with the amino acid sequence shown in any one of SEQ ID NO: 78-88, wherein the C-terminus of the heavy chain variable region is connected to the N-terminus of the light chain variable region through a connecting peptide linker, or the C-terminus of the light chain variable region is connected to the N-terminus of the heavy chain variable region through a connecting peptide linker.

39. The antibody or antigen-binding fragment thereof of claim 37, wherein the small molecule antibody comprises at least one of Fab antibody, Fv antibody, single domain antibody and minimum recognition unit.

40. A nucleic acid molecule encoding the antibody or antigen-binding fragment thereof of claim 29.

41. The nucleic acid molecule of claim 40, wherein the nucleic acid molecule has a nucleotide sequence shown in any one of SEQ ID NO: 113-123 or a nucleotide sequence shown in any one of SEQ ID NO: 124-134.

42. An expression vector carrying the nucleic acid molecule of claim 40.

43. A recombinant cell expressing the antibody or antigen-binding fragment thereof of claim 29, or carrying the nucleic acid molecule encoding the antibody or antigen-binding fragment.

44. A pharmaceutical composition comprising the antibody or antigen-binding fragment thereof of claim 29, or the nucleic acid molecule encoding the antibody or antigen-binding fragment, or the expression vector carrying the nucleic acid molecule, or the recombinant cell expressing the antibody or antigen-binding fragment, or the recombinant cell carrying the nucleic acid molecule.

45. A method of treating or preventing pulmonary fibrosis, non-alcoholic steatohepatitis, chronic heart failure or diseases caused by the transformation of fibroblasts into fibrosis in a subject comprising administering to the subject a therapeutically effective amount of claim 29, or the nucleic acid molecule encoding the antibody, or the expression vector carrying the nucleic acid molecule, or the recombinant cell expressing the antibody, or the recombinant cell carrying the nucleic acid molecule.

46. A method of treating or preventing pulmonary fibrosis, non-alcoholic steatohepatitis, chronic heart failure or diseases caused by the transformation of fibroblasts into fibrosis in a subject comprising administering to the subject a therapeutically effective amount of the pharmaceutical composition of claim 44.

47. A kit for detecting IL-11 comprising the antibody of claim 29.

48. A method of detecting IL-11 or diagnosing IL-11-related diseases in a subject using a kit comprising the antibody of claim 29, or the nucleic acid molecule encoding the antibody, or the expression vector carrying the nucleic acid molecule, or the recombinant cell expressing the antibody, or the recombinant cell carrying the nucleic acid molecule.
